SEQUENCE LISTING (90 sequences)

SEQ ID NO: 1 - DNA, 40 bp, Artificial Sequence
  pSIM1278 construct junction LB Synthetic-LB Potato
tggcaggatatataccggtgtaaacgaagtgtgtgtggtt

SEQ ID NO: 2 - DNA, 40 bp, Artificial Sequence
  pSIM1278 construct junction RB Synthetic-RB Potato
gtttacagtaccatatatcctgtcagaggtatagaggcat

SEQ ID NO: 3 - DNA, 36 bp, Artificial Sequence
  pSIM1278 construct junction LB-AGP
ttgtggaggagtaagggtaccaagtgtctgagacaa

SEQ ID NO: 4 - DNA, 30 bp, Artificial Sequence
  pSIM1278 construct junction AGP-ASN
aacaagcttgttaacgaactctttatccag

SEQ ID NO: 5 - DNA, 30 bp, Artificial Sequence
  pSIM1278 construct junction ASN-PPO
tggtgagccctcgagataattgtaactgat

SEQ ID NO: 6 - DNA, 36 bp, Artificial Sequence
  pSIM1278 construct junction PPO-Spacer1
attcaatagagactatctagagtgtatgggtgatcc

SEQ ID NO: 7 - DNA, 30 bp, Artificial Sequence
  pSIM1278 construct junction Spacer1-PPO
ttatggaggaatcagtagtctctattgaat

SEQ ID NO: 8 - DNA, 30 bp, Artificial Sequence
  pSIM1278 construct junction PPO-ASN
atcagttacaattattctcgagggctcacc

SEQ ID NO: 9 - DNA, 36 bp, Artificial Sequence
  pSIM1278 construct junction ASN-GBSS
ctggataaagagttcgaattcgtgatgtgtggtcta

SEQ ID NO: 10 - DNA, 46 bp, Artificial Sequence
  pSIM1278 construct junction GBSS-AGP
tgagatgcatggttcactagtgattggtaccaagtgtctgagacaa

SEQ ID NO: 11 - DNA, 30 bp, Artificial Sequence
  pSIM1278 construct junction AGP-PHL
aacaagcttgttaacgtgctctctatgcaa

SEQ ID NO: 12 - DNA, 30 bp, Artificial Sequence
  pSIM1278 construct junction PHL-R1
ttttgctaaaacatcggacacgtatatttt

SEQ ID NO: 13 - DNA, 36 bp, Artificial Sequence
  pSIM1278 construct junction R1-Spacer2
atatttattatataactgcagcgttgttttgatgaa

SEQ ID NO: 14 - DNA, 30 bp, Artificial Sequence
  pSIM1278 construct junction Spacer2-R1
ttttgatgaaaaagcttatataataaatat

SEQ ID NO: 15 - DNA, 30 bp, Artificial Sequence
  pSIM1278 construct junction PHL-GBSS
ttgcatagagagcaccgtgatgtgtggtct

SEQ ID NO: 16 - DNA, 39 bp, Artificial Sequence
  pSIM1278 construct junction GBSS-RB
aaatcataatatctggagctcatcgttatgctataaatt

SEQ ID NO: 17 - DNA, 30 bp, Artificial Sequence
  E12 specific junction LJ-plant-insert
tattggtgagaataataaacgaagtgtgtg

SEQ ID NO: 18 - DNA, 30 bp, Artificial Sequence
  E12 specific junction RJ  insert-plant
acggtcagtcactttgtactagtaaagatc

SEQ ID NO: 19 - DNA, 36 bp, Artificial Sequence
  F10 specific junction LJ-plant-insert
cagcagccatcaactggtccagcaggatatataccg

SEQ ID NO: 20 - DNA, 30 bp, Artificial Sequence
  F10 specific junction RJ  insert-plant
gtgggcatcaatttagtccaccagccaaaa

SEQ ID NO: 21 - DNA, 35 bp, Artificial Sequence
  J3 specific junction LJ-plant-insert
aacaggacaaccacaagctaggaaactcacatgct

SEQ ID NO: 22 - DNA, 30 bp, Artificial Sequence
  J3 specific junction RJ  insert-plant
aaaaagccctagaattaattttttttggaa

SEQ ID NO: 23 - DNA, 30 bp, Artificial Sequence
  J3 specific junction internal
gaactgaaaccgatatacaaaatggtataa

SEQ ID NO: 24 - DNA, 30 bp, Artificial Sequence
  J3 specific junction internal
cctctatacctctgacagaggtatagaggc

SEQ ID NO: 25 - DNA, 30 bp, Artificial Sequence
  J55 specific junction LJ-plant-insert
aaatcaacaagcaattgaagaactacctat

SEQ ID NO: 26 - DNA, 30 bp, Artificial Sequence
  J55 specific junction RJ  insert-plant
cataagtgagactatgcgtgaaactttcag

SEQ ID NO: 27 - DNA, 30 bp, Artificial Sequence
  J55 specific junction internal
cctctatacctctgataaattaaactgact

SEQ ID NO: 28 - DNA, 30 bp, Artificial Sequence
  J55 specific junction internal
actaaaaatctcagctaaaacaataaaaat

SEQ ID NO: 29 - DNA, 30 bp, Artificial Sequence
  V11 specific junction LJ  plant-insert
tcatctttcattccggtgtaaacgaagtgt

SEQ ID NO: 30 - DNA, 30 bp, Artificial Sequence
  V11 specific junction RJ  insert-plant
atgcctctatacctctgataataaagaact

SEQ ID NO: 31 - DNA, 30 bp, Artificial Sequence
  W8 specific junction LJ  plant-pSIM1678 insert
caacaattaaaaaaagcctaattttcatgg

SEQ ID NO: 32 - DNA, 30 bp, Artificial Sequence
  W8 specific junction RJ  pSIM1678 insert-plant
acggtcagtcactttcaagccctaggagcc

SEQ ID NO: 33 - DNA, 30 bp, Artificial Sequence
  W8 specific junction LJ  plant-pSIM1278 insert
agccctacaaaaggcccaaactctaagtca

SEQ ID NO: 34 - DNA, 30 bp, Artificial Sequence
  W8 specific junction RJ  plant-pSIM1278 insert
agtcctagaactacgaaaattatattcggt

SEQ ID NO: 35 - DNA, 30 bp, Artificial Sequence
  W8 specific junction internal
atgcctctatacctcagaggtatagaggca

SEQ ID NO: 36 - DNA, 30 bp, Artificial Sequence
  W8 specific junction internal
tgcctctatacctctgacgcaacacaacac

SEQ ID NO: 37 - DNA, 30 bp, Artificial Sequence
  X17 specific junction LJ  plant-insert
gtattcgctgcaggatatataccggtgtaa

SEQ ID NO: 38 - DNA, 30 bp, Artificial Sequence
  X17 specific junction RJ  insert-plant
gcctctatacctctgactggaaaattgacg

SEQ ID NO: 39 - DNA, 30 bp, Artificial Sequence
  Y9 specific junction LJ  plant-insert
caaaatgtgcaccaatgatgtctcacgacc

SEQ ID NO: 40 - DNA, 30 bp, Artificial Sequence
  Y9 specific junction RJ  insert-plant
ctcaacaaccagtcaccggaagtcccttaa

SEQ ID NO: 41 - DNA, 30 bp, Artificial Sequence
  Y9 specific junction internal
atgcctctatacctctgaaagtgactgacc

SEQ ID NO: 42 - DNA, 36 bp, Artificial Sequence
  pSIM1678 construct junction LB-pVnt1
ttgtggaggagtaagggtaccagttatacaccctac

SEQ ID NO: 43 - DNA, 30 bp, Artificial Sequence
  pSIM1678 construct junction pVNT1-Vnt1
accagctaacaaaagatgaattattgtgtt

SEQ ID NO: 44 - DNA, 30 bp, Artificial Sequence
  pSIM1678 construct junction Vnt1-tVnt1
tcacaggtactataaataattatttacgtt

SEQ ID NO: 45 - DNA, 36 bp, Artificial Sequence
  pSIM1678 construct junction tVnt1-pAGP
agcctcttttcaaaggggccccaagtgtctgagaca

SEQ ID NO: 46 - DNA, 36 bp, Artificial Sequence
  pSIM1678 construct junction pAGP-Inv
aacaagcttgttaacggatccacattcctcccggat

SEQ ID NO: 47 - DNA, 36 bp, Artificial Sequence
  pSIM1678 construct junction Inv-Inv
tcaaggactttagaggaattcagcggacccagtcca

SEQ ID NO: 48 - DNA, 36 bp, Artificial Sequence
  pSIM1678 construct junction Inv-pGbss
gaggaatgtgtaatgactagtcgtgatgtgtggtct

SEQ ID NO: 49 - DNA, 20 bp, Artificial Sequence
  Adenine Phosphoribosyl Transferase APRT forward primer
gaaccggagcaggtgaagaa

SEQ ID NO: 50 - DNA, 20 bp, Artificial Sequence
  Adenine Phosphoribosyl Transferase APRT reverse primer
gaagcaatcccagcgatacg

SEQ ID NO: 51 - DNA, 17 bp, Artificial Sequence
  Adenine Phosphoribosyl Transferase APRT probe
cgcctcatgatccgatt

SEQ ID NO: 52 - DNA, 18 bp, Artificial Sequence
  E12 forward primer
gggagtaggctttatacc

SEQ ID NO: 53 - DNA, 24 bp, Artificial Sequence
  E12 reverse primer
ccttgaatgatttgatataagaag

SEQ ID NO: 54 - DNA, 18 bp, Artificial Sequence
  E12 probe
tcagtcactttgtactag

SEQ ID NO: 55 - DNA, 19 bp, Artificial Sequence
  F10 forward primer
aaggcaatctttcataagc

SEQ ID NO: 56 - DNA, 18 bp, Artificial Sequence
  F10 reverse primer
cacacacttcgtttacac

SEQ ID NO: 57 - DNA, 17 bp, Artificial Sequence
  F10 probe
tatatcctgctggacca

SEQ ID NO: 58 - DNA, 19 bp, Artificial Sequence
  J3 forward primer
ccggtactcaaatttgcaa

SEQ ID NO: 59 - DNA, 23 bp, Artificial Sequence
  J3 reverse primer
ggtgttcttaatatcgagtgttc

SEQ ID NO: 60 - DNA, 19 bp, Artificial Sequence
  J3 probe
ccacaagctaggaaactca

SEQ ID NO: 61 - DNA, 23 bp, Artificial Sequence
  J55 forward primer
catggaattatttagaaacaaaa

SEQ ID NO: 62 - DNA, 20 bp, Artificial Sequence
  J55 reverse primer
gacgatcaagaatctcaata

SEQ ID NO: 63 - DNA, 19 bp, Artificial Sequence
  J55 probe
tcatagtcaaacagtcagc

SEQ ID NO: 64 - DNA, 18 bp, Artificial Sequence
  V11 forward primer
gtgggcatcaatttagtg

SEQ ID NO: 65 - DNA, 20 bp, Artificial Sequence
  V11 reverse primer
ggagagtaatagccttagta

SEQ ID NO: 66 - DNA, 19 bp, Artificial Sequence
  V11 probe
cctctatacctctgataat

SEQ ID NO: 67 - DNA, 18 bp, Artificial Sequence
  V11 forward primer
tagccaagcatgacttac

SEQ ID NO: 68 - DNA, 18 bp, Artificial Sequence
  V11 reverse primer
cacacacttcgtttacac

SEQ ID NO: 69 - DNA, 18 bp, Artificial Sequence
  V11 probe
atgtatgaatggcagaag

SEQ ID NO: 70 - DNA, 18 bp, Artificial Sequence
  W8 forward primer
aggctttataccgagttg

SEQ ID NO: 71 - DNA, 22 bp, Artificial Sequence
  W8 reverse primer
tcagcttaatcgaataagaaac

SEQ ID NO: 72 - DNA, 17 bp, Artificial Sequence
  W8 probe
actacggtcagtcactt

SEQ ID NO: 73 - DNA, 18 bp, Artificial Sequence
  X17 forward primer
gtccgaaggttgagaaaa

SEQ ID NO: 74 - DNA, 20 bp, Artificial Sequence
  X17 reverse primer
cgtgagacatcatcataatg

SEQ ID NO: 75 - DNA, 17 bp, Artificial Sequence
  X17 probe
cgcttcagttacggctt

SEQ ID NO: 76 - DNA, 25 bp, Artificial Sequence
  Y9 forward primer
gacagctatcaatatactttagtaa

SEQ ID NO: 77 - DNA, 19 bp, Artificial Sequence
  Y9 reverse primer
ttgccatactcatacagag

SEQ ID NO: 78 - DNA, 19 bp, Artificial Sequence
  Y9 probe
aatgatgtctcacgaccaa

SEQ ID NO: 79 - DNA, 19 bp, Artificial Sequence
  pSIM1278 forward primer
tcaccagtatgactgttta

SEQ ID NO: 80 - DNA, 18 bp, Artificial Sequence
  pSIM1278 reverse primer
ctaccacacactctctag

SEQ ID NO: 81 - DNA, 19 bp, Artificial Sequence
  pSIM1278 probe
aagcttgttaacgaactct

SEQ ID NO: 82 - DNA, 18 bp, Artificial Sequence
  pSIM1278 forward primer
gcatcatccatcaaagtg

SEQ ID NO: 83 - DNA, 21 bp, Artificial Sequence
  pSIM1278 reverse primer
gtcggtataataagagaagga

SEQ ID NO: 84 - DNA, 19 bp, Artificial Sequence
  pSIM1278 probe
tagagactatctagagtgt

SEQ ID NO: 85 - DNA, 18 bp, Artificial Sequence
  pSIM1678 forward primer
gaacggagggagtatgaa

SEQ ID NO: 86 - DNA, 19 bp, Artificial Sequence
  pSIM1678 reverse primer
ccactttcagttttggttg

SEQ ID NO: 87 - DNA, 17 bp, Artificial Sequence
  pSIM1678 probe
tcttttcaaaggggccc

SEQ ID NO: 88 - DNA, 18 bp, Artificial Sequence
  pSIM1678 forward primer
gaagcctcttttcaaagg

SEQ ID NO: 89 - DNA, 18 bp, Artificial Sequence
  pSIM1678 reverse primer
acgggttaatcgataacc

SEQ ID NO: 90 - DNA, 16 bp, Artificial Sequence
  pSIM1678 probe
ccccaagtgtctgaga

(19) United States
(12) Patent Application Publication
(10) Pub. No.: US 2016/0102371 A1
(43) Pub. Date: Apr. 14, 2016

(54) EVENT-SPECIFIC DETECTION METHODS

(71) Applicant: J.R. SIMPLOT COMPANY, Boise, ID (US)

(72) Inventors: Jingsong Ye, Boise, ID (US); Jeffrey W. Habig, Boise, ID (US); Janet Layne, Meridian, ID (US); Jeffery W. Hein, Boise, ID (US); Matthew G. Pence, Eagle, ID (US); Stephanie Hudon, Meridian, ID (US)

(73) Assignee: J.R. SIMPLOT COMPANY, Boise, ID (US)

(21) Appl. No.: 14/879,365

(22) Filed: Oct. 9, 2015

Related U.S. Application Data
(60) Provisional application No. 62/118,320, filed on Feb. 19, 2015.
(60) Provisional application No. 62/062,324, filed on Oct. 10, 2014.

(51) Int. Cl. C12Q 1/68

(52) U.S. Cl. CPC C12Q 1/6895; C12Q 2600/156; C12Q 2600/13

(57) ABSTRACT

The present disclosure concerns methods for identifying genetic material in recombinant potato plants, including in food products made from such plants. The disclosure relates to the materials, including nucleotide primers and probes, utilized in the methods set forth herein. Furthermore, the disclosure provides for non-naturally occurring nucleotide junction sequences per se that result from genetic recombination events and methods of detecting said junction sequences.

### CROSS-REFERENCE TO RELATED APPLICATIONS

The present application claims the benefit of priority to U.S. Provisional Patent Application No. 62/062,324, filed on Oct. 10, 2014, and U.S. Provisional Patent Application No. 62/118,320, filed on Feb. 19, 2015, the entire contents of each of which are hereby incorporated by reference in their entirety for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: JRSI_062_02US_SeqList_ST25.txt, date recorded: Oct. 4, 2015, file size≈26 kilobytes).

### FIELD

The present disclosure concerns methods for identifying genetic material in recombinant potato plants, including in food products made from such plants. Furthermore, the disclosure relates to the materials—including nucleotide primers, probes, and the non-naturally occurring nucleotide junction sequences per se—utilized in the methods set forth herein.

### BACKGROUND

The potato is the world's fourth most important food crop and by far the most important vegetable. Potatoes are currently grown commercially in nearly every state of the United States. Annual potato production exceeds 18 million tons in the United States and 300 million tons worldwide. The popularity of the potato derives mainly from its versatility and nutritional value. Potatoes can be used fresh, frozen or dried, or can be processed into flour, starch or alcohol. They contain complex carbohydrates and are rich in calcium, niacin and vitamin C.

The quality of potatoes in the food industry is affected by two critical factors: (1) potatoes contain large amounts of asparagine, a non-essential free amino acid that is rapidly oxidized to form acrylamide, a carcinogenic product, upon frying or baking; and (2) potatoes are highly susceptible to enzymatic browning and discoloration, an undesirable event which happens when polyphenol oxidase leaks out from the damaged plastids of bruised potatoes. In the cytoplasm, the enzyme oxidizes phenols, which then rapidly polymerize to produce dark pigments. Tubers contain large amounts of phosphorylated starch, some of which is degraded during storage to produce glucose and fructose. These reducing sugars react with amino acids to form Maillard products, including acrylamide, when heated at temperatures above 120° C. Two enzymes involved in starch phosphorylation are water dikinase R1 and phosphorylase-L (R1 and PhL). Browning is also triggered non-enzymatically as a consequence of the partial degradation of starch into glucose and fructose.

In order to address the two aforementioned factors, potato plant varieties that produce tubers with low acrylamide content, increased black spot bruise tolerance, and reduced levels of reducing sugars have now been developed (U.S. Pat. No. 8,754,303, “Potato Cultivar J3”; U.S. Pat. No. 8,710,311 “Potato Cultivar F10”; U.S. Pat. No. 8,889,963 “Potato Cultivar J55”; U.S. patent application Ser. No. 14/072,487 “Potato Cultivar E12”; and U.S. Pat. No. 8,889,964 “Potato Cultivar W8,” which is also resistant to late blight, each of which is hereby incorporated by reference in their entirety). These modified potato plants have been developed by the introduction of genetic events, without foreign nucleic acids from a bacterium being introduced into the potato.

However, there is an important need in the industry to have methods and materials for identifying introduced genetic material in these modified potato plants, including in food products made from such plants. Specifically, there is a need to have methods and materials to determine whether a given potato, or potato product, contains a particular introduced genetic transformation event.

### SUMMARY OF THE DISCLOSURE

The present disclosure provides methods of identifying genetic transformation events in a plant. In some embodiments, the plant is a potato. The methods set forth herein are broadly applicable to detecting non-naturally occurring nucleotide junctions that result from plant transformation. The plant transformation events, which in some aspects occur through Agrobacterium mediated vectors, create unique non-naturally occurring nucleotide junction sequences that can be detected by the present methods. These transformation events may be detected in any plant species; however, in certain exemplified embodiments, the methods taught herein teach the detection of non-naturally occurring nucleotide junctions in eight potato cultivars.

In some aspects, the disclosure provides methods and materials that are able to detect transformed potatoes that comprise inserted nucleic acid sequences that are native to the potato plant genome and do not contain, Agrobacterium DNA, viral markers, or vector backbone sequences. Rather, the DNA that is inserted into the genome of the potato varieties, and which is detected by embodiments of the methods described herein, can be non-coding polynucleotides that are native to potato, or native to wild potato, or native to a potato sexually-compatible plant. These introduced nucleotides function to silence genes involved in the expression of black spot bruises, asparagine accumulation, and reducing sugar accumulation. Consequently, these introduced genes lead to lower acrylamide content in the transformed potatoes. Furthermore, the methods taught herein are able to detect genes, and in some aspects coding polynucleotides, introduced into a potato that confer resistance to late blight. The aforementioned inserted DNA creates unique non-naturally occurring nucleotide junctions that are not found in nature.

Thus, the transformation events taught herein lead to the creation of non-natural nucleotide “junction” sequences in the transformed potato. These non-naturally occurring nucleotide junctions can be used as a type of diagnostic that is indicative of the presence of particular genetic transformation events. As aforementioned, the methods taught herein are not limited to potatoes. Rather, the eight potato cultivars utilized herein demonstrate the applicability of the present methods of detecting non-naturally occurring nucleotide junction sequences resultant from plant transformation, in any plant species.

The present techniques are able to detect these non-naturally occurring nucleotide junctions via the utilization of specialized quantitative PCR methods, including uniquely designed primers and probes. In some aspects, the probes of the disclosure bind to the non-naturally occurring nucleotide junction sequences. In some aspects, traditional PCR is utilized. In other aspects, real-time PCR is utilized. In some aspects, quantitative PCR (qPCR) is utilized.

Thus, the disclosure covers the utilization of two common methods for the detection of PCR products in real-time: (1) non-specific fluorescent dyes that intercalate with any double-stranded DNA, and (2) sequence-specific DNA probes consisting of oligonucleotides that are labelled with a fluorescent reporter which permits detection only after hybridization of the probe with its complementary sequence. In some aspects, only the non-naturally occurring nucleotide junction will be amplified via the taught primers, and consequently can be detected via either a non-specific dye, or via the utilization of a specific hybridization probe.

Furthermore, by creating non-naturally occurring nucleotide junction sequences—resultant from the disclosed transformation events—the present inventors have created unique nucleotide sequences that are not found in nature. These sequences can be isolated and comprise a nucleotide molecule that does no exist in nature without the hand of man intervening to create such a molecule. Furthermore, the disclosed probe sequences, which bind to the non-naturally occurring nucleotide junction sequences, are also novel nucleotide molecules that are not found in nature. Consequently, aspects of the disclosure involve non-naturally occurring nucleotide junction sequence molecules per se, along with other nucleotide molecules that are capable of binding to said non-naturally occurring nucleotide junction sequences under mild to stringent hybridization conditions. In some aspects, the nucleotide molecules that are capable of binding to said non-naturally occurring nucleotide junction sequences under mild to stringent hybridization conditions are termed “nucleotide probes.”

Thus, in the present disclosure, representative methods of detecting the E12, F10, J3, J55, V11, W8, X17, and Y9 genetic transformation events are described. The disclosure provides for methods of detecting non-naturally occurring nucleotide junction sequences that result from the aforementioned transformation events. These eight examples serve as species that enable the larger genus of detecting non-naturally occurring nucleotide junction sequences resultant from a transformation event, in any plant species.

In one embodiment, a quantitative PCR method for detecting the presence of a plant transformation event in a nucleic acid sample is provided, comprising: a) combining: i) a pair of forward and reverse nucleotide primers, ii) a nucleotide probe, and iii) a target nucleotide sequence from said sample comprising a non-naturally occurring nucleotide junction to be detected; wherein the nucleotide probe binds to the non-naturally occurring nucleotide junction, or a sequence indicative of the presence of the non-naturally occurring nucleotide junction; and b) detecting the target nucleotide sequence from said sample.

In one aspect, the non-naturally occurring nucleotide junction results from a plant transformation event selected from the group consisting of: E12, F10, J3, J55, V11, W8, X17, and Y9, or combinations thereof. In some aspects, the non-naturally occurring nucleotide junctions sequences are found in food product material. In particular aspects, the food product material is a potato food product material.

In one aspect, the target nucleotide sequence comprises at least one nucleotide sequence selected from the group consisting of: SEQ ID NOs: 1-48.

In one aspect, the pair of forward and reverse nucleotide primers and the nucleotide probe are selected from the group consisting of SEQ ID NOs: 52-90.

In one embodiment, the forward nucleotide primer comprises SEQ ID NO: 52 and the reverse nucleotide primer comprises SEQ ID NO: 53 and the nucleotide probe comprises SEQ ID NO: 54 and the nucleotide probe binds to the non-naturally occurring nucleotide junction.

In one embodiment, the nucleotide probe binds the left or right junction of an E12 event.

In one embodiment, the forward nucleotide primer comprises SEQ ID NO: 55 and the reverse nucleotide primer comprises SEQ ID NO: 56 and the nucleotide probe comprises SEQ ID NO: 57 and the nucleotide probe binds to the non-naturally occurring nucleotide junction.

In one embodiment, the nucleotide probe binds the left or right junction of an F10 event.

In one embodiment, the forward nucleotide primer comprises SEQ ID NO: 58 and the reverse nucleotide primer comprises SEQ ID NO: 59 and the nucleotide probe comprises SEQ ID NO: 60 and the nucleotide probe binds to the non-naturally occurring nucleotide junction.

In one embodiment, the nucleotide probe binds the left or right junction of a J3 event.

In one embodiment, the forward nucleotide primer comprises SEQ ID NO: 61 and the reverse nucleotide primer comprises SEQ ID NO: 62 and the nucleotide probe comprises SEQ ID NO: 63 and the nucleotide probe binds to the sequence indicative of the presence of the non-naturally occurring nucleotide junction present in a J55 event.

In one embodiment, the forward nucleotide primer comprises SEQ ID NO: 64 or 67 and the reverse nucleotide primer comprises SEQ ID NO: 65 or 68 and the nucleotide probe comprises SEQ ID NO: 66 or 69 and the nucleotide probe binds to the non-naturally occurring nucleotide junction.

In one embodiment, the nucleotide probe binds the left or right junction of a V11 event.

In one embodiment, the forward nucleotide primer comprises SEQ ID NO: 70 and the reverse nucleotide primer comprises SEQ ID NO: 71 and the nucleotide probe comprises SEQ ID NO: 72 and the nucleotide probe binds to the sequence indicative of the presence of the non-naturally occurring nucleotide junction present in a W8 event.

In one embodiment, the forward nucleotide primer comprises SEQ ID NO: 73 and the reverse nucleotide primer comprises SEQ ID NO: 74 and the nucleotide probe comprises SEQ ID NO: 75 and the nucleotide probe binds to the non-naturally occurring nucleotide junction.

In one embodiment, the nucleotide probe binds the left or right junction of an X17 event.

In one embodiment, the forward nucleotide primer comprises SEQ ID NO: 76 and the reverse nucleotide primer comprises SEQ ID NO: 77 and the nucleotide probe comprises SEQ ID NO: 78 and the nucleotide probe binds to the non-naturally occurring nucleotide junction.

In one embodiment, the nucleotide probe binds the left or right junction of a Y9 event.

In one embodiment, the forward nucleotide primer comprises SEQ ID NO: 79 and the reverse nucleotide primer comprises SEQ ID NO: 80 and the nucleotide probe comprises SEQ ID NO: 81 and the nucleotide probe binds to the non-naturally occurring nucleotide junction.

In one embodiment, the nucleotide probe binds an internal AGP/Asn1 junction associated with pSIM1278.

In one embodiment, the forward nucleotide primer comprises SEQ ID NO: 82 and the reverse nucleotide primer comprises SEQ ID NO: 83 and the nucleotide probe comprises SEQ ID NO: 84 and the nucleotide probe binds to the non-naturally occurring nucleotide junction.

In one embodiment, the nucleotide probe binds an internal junction associated with pSIM1278.

In one embodiment, the forward nucleotide primer comprises SEQ ID NO: 85 and the reverse nucleotide primer comprises SEQ ID NO: 86 and the nucleotide probe comprises SEQ ID NO: 87 and the nucleotide probe binds to the non-naturally occurring nucleotide junction.

In one embodiment, the nucleotide probe binds an internal junction associated with pSIM1678. In a particular aspect, the nucleotide probe binds to an internal Vnt1 terminator/pAgp junction

In one embodiment, the forward nucleotide primer comprises SEQ ID NO: 88 and the reverse nucleotide primer comprises SEQ ID NO: 89 and the nucleotide probe comprises SEQ ID NO: 90 and the nucleotide probe binds to the non-naturally occurring nucleotide junction.

In one embodiment, the nucleotide probe binds an internal junction associated with pSIM1678.

In one embodiment, a non-naturally occurring construct-specific junction sequence associated with pSIM1278 or pSIM1678 is detected. In these embodiments, the non-naturally occurring junction sequences illustrated in both Table 6 “pSIM1278 and pSIM1678 construct junctions” and FIG. 5 are detected.

In one embodiment, a non-naturally occurring event-specific junction sequence associated with event E12, F10, J3, J55, V11, W8, X17, or Y9 is detected. In these embodiments, the non-naturally occurring junction sequences illustrated in both Table 6 and FIG. 6 are detected.

In one embodiment, the nucleic acid sample is from a potato plant, or potato plant part, or potato derived food product, or potato based ingredient utilized in a food product.

In one embodiment, the potato plant part is at least one selected from the group consisting of: potato flowers, potato tepals, potato petals, potato sepals, potato anthers, potato pollen, potato seeds, potato leaves, potato petioles, potato stems, potato roots, potato rhizomes, potato stolons, potato tubers, potato shoots, potato cells, potato protoplasts, potato plant tissues, and combinations thereof.

In one embodiment, the potato derived food product is at least one selected from the group consisting of: a potato processed food product, a potato livestock feed material, French fries, potato chips, dehydrated potato material, potato flakes, potato granules, potato protein, potation flour, and combinations thereof.

In one embodiment, the nucleic acid sample is from a potato derived food product and wherein the presence of at least one plant transformation event selected from the group consisting of: E12, F10, J3, J55, V11, W8, X17, Y9, or combinations thereof, is able to be detected in the food product.

In one embodiment, the transformation event is able to be detected at levels less than 20%, less than 10%, less than 5%, less than 1%, and less than 0.5% of the total food product. In one embodiment, the transformation event is able to be detected at levels ranging from about 0.1% to about 5% of the total food product, or at levels ranging from about 0.2% to about 5.0% of the total food product, or at levels ranging from about 0.1% to about 10% of the total food product.

In one embodiment, an isolated non-naturally occurring nucleic acid junction sequence sharing at least 75%, 80%, 85%, 90%, 91%, 92%, 93%, 94%, 95%, 96%, 97%, 98%, 99%, or 100% sequence homology to a nucleic acid selected from the group consisting of SEQ ID NOs: 1-48 is provided.

In another embodiment, an isolated non-naturally occurring nucleic acid junction sequence sharing at least 75%, 80%, 85%, 90%, 91%, 92%, 93%, 94%, 95%, 96%, 97%, 98%, 99%, or 100% sequence homology to a non-naturally occurring nucleotide junction sequence created by the insertion of pSIM1278 and/or pSIM1678 into a potato is provided. In some aspects, the non-naturally occurring nucleotide junction sequences are depicted in FIG. 5 (construct specific junctions) and FIG. 6 (event specific junctions) and Table 6.

In another embodiment, an isolated non-naturally occurring nucleic acid junction sequence selected from the group consisting of SEQ ID NOs: 1-48 is provided. In one embodiment, the non-natural nucleotide junction sequences are found in Table 6. The Table 6 also includes an indication of whether or not the junction sequences are contained on the “left” or “right” of the genetic insert. In some aspects, these non-natural nucleotide junction sequences are comprised in longer nucleotide sequences. For instance, the sequences in Table 6 can be included in nucleotide sequences comprising 30, 40, 50, 60, 70, 80, 90, 100, 200, 300, 400, 500, or more nucleotides in length.

In one embodiment, an isolated non-naturally occurring nucleic acid sequence capable of hybridizing under mild conditions to a nucleic acid selected from the group consisting of SEQ ID NOs: 1-48 is provided. In one embodiment, an isolated non-naturally occurring nucleic acid sequence capable of hybridizing under stringent conditions to a nucleic acid selected from the group consisting of SEQ ID NOs: 1-48 is provided. In some aspects, the aforementioned nucleic acid sequence capable of hybridizing to the SEQ ID NOs: 1-48 is a nucleotide probe. In some aspects, the nucleotide probe is configured for real-time PCR. In some aspects, the probe is labeled with a reporter molecule.

In another embodiment, an isolated non-naturally occurring nucleic acid probe sequence sharing at least 75%, 80%, 85%, 90%, 91%, 92%, 93%, 94%, 95%, 96%, 97%, 98%, 99%, or 100% sequence homology to a nucleic acid selected from the group consisting of SEQ ID NOs: 54, 57, 60, 63, 66, 69, 72, 75, 78, 81, 84, 87, and 90 is provided. In another embodiment, an isolated non-naturally occurring nucleic acid probe sequence selected from the group consisting of SEQ ID NOs: 54, 57, 60, 63, 66, 69, 72, 75, 78, 81, 84, 87, and 90 is provided. As set forth in Table 7, the aforementioned probes are able to bind to non-naturally occurring even-specific and construct-specific nucleotide junction sequences as follows: 54 (E12), 57 (F10), 60 (J3), 63 (J55), 66 (V11), 69 (V11), 72 (W8), 75 (X17), 78 (Y9), 81 (pSIM1278), 84 (pSIM1278), 87 (pSIM1678), and 90 (pSIM1678).

In another embodiment, an isolated non-naturally occurring nucleic acid primer or probe sequence sharing at least 75%, 80%, 85%, 90%, 91%, 92%, 93%, 94%, 95%, 96%, 97%, 98%, 99%, or 100% sequence homology to a nucleic acid selected from the group consisting of SEQ ID NOs: 52-90 is provided. In another embodiment, an isolated non-naturally occurring nucleic acid primer or probe sequence selected from the group consisting of SEQ ID NOs: 52-90 is provided.

Also taught herein are kits comprising the forward and reverse nucleotide primers and nucleotide probes according to the aforementioned disclosure, along with standard reagents utilized in PCR and qPCR reactions.

Further, in one embodiment, the present disclosure provides a plant vector, referred to as pSIM1278, that comprises: a first silencing cassette containing two copies of a DNA segment comprising, in anti-sense orientation, a fragment of the asparagine synthetase-1 gene (fAsn1) and the 3′-untranslated sequence of the polyphenol oxidase-5 gene; and a second silencing cassette containing two copies of a DNA segment comprising, in anti-sense orientation, a fragment of the potato phosphorylase-L (pPhL) gene and a fragment of the potato R1 gene.

The pSIM1278 vector comprises a 9,512 bp backbone region that supports maintenance of the plant DNA prior to plant transformation and is not transferred into plant cells upon transformation of the plant cells, and a 10,148 bp DNA insert region comprising native DNA that is stably integrated into the genome of the plant cells upon transformation.

Further, in another embodiment, the present disclosure provides a plant vector, referred to as pSIM1678, that comprises: a first expression cassette containing one copy of a DNA segment comprising, in sense orientation, an Rpi-vnt1 late blight resistance gene (Vnt1); and a second silencing cassette containing two copies of a DNA segment comprising, in anti-sense orientation, a fragment of the vacuolar acid invertase (VInv) gene.

The pSIM1678 vector comprises a 9,512 bp backbone region that supports maintenance of the plant DNA prior to plant transformation and is not transferred into plant cells upon transformation of the plant cells, and a 9,090 bp DNA insert region comprising native DNA that is stably integrated into the genome of the plant cells upon transformation.

The disclosure provides methods of detecting whether or not the aforementioned DNA insert region has been introduced into a plant. As aforementioned, the inserted region of DNA leads to the formation of unique non-natural nucleotide junction sequences. These junction sequences can be found in Table 6, FIG. 5, and FIG. 6, amongst other places of the disclosure.

Furthermore, the disclosure can detect whether or not the pSIM1278 and/or pSIM1678 vector was utilized to introduce genetic material into a plant. In some embodiments, the plant is a potato. However, the present methods and materials disclosed herein could be used to detect the presence of the taught genetic events introduced into any suitable plant. As the present methods can be utilized to detect whether or not the pSIM1278 and/or pSIM1678 vector was utilized to introduce genetic material into a plant, the methods can detect transformation in any plant, or any potato plant, which utilized such vectors to introduce DNA into the plant.

In embodiments, methods were developed to optimize DNA extraction for the purpose of providing event specific detection of biotech potato food products.

Presented in certain embodiments herein, are a detailed description of all equipment, reagents, and methods used in the taught event-specific, or construct-specific, detection protocols. In addition, data are presented, in certain aspects, to support the repeatability of the taught PCR DNA extraction procedures along with screening for DNA quality.

In some of the taught aspects of PCR event-specific, or construct-specific detection, all procedures for Real-Time PCR are outlined, along with evidence of the ability to detect low levels (0.2-5.0%) of biotech potatoes in food products.

In one aspect of the disclosure, the potato plant variety expressing one or more of the silencing cassettes of the plant DNA vector is selected from the group consisting of the following transformation events: E12 (Russet Burbank), J3 (Atlantic), J55 (Atlantic), F10 (Ranger Russet), W8 (Russet Burbank), V11 (Snowden), X17 (Ranger Russet), and Y9 (Atlantic). The disclosure teaches methods and materials useful for detecting the presence of any of the aforementioned genetic transformation events. These events are able to be detected utilizing a part of a potato plant.

In specific embodiments, any part of a potato plant can be utilized to isolate genetic material for incorporation into the detection methods taught herein. In some aspects, the taught methods will utilize embryos, protoplasts, meristematic cells, callus, pollen, leaves, anthers, pistils, cotyledons, hypocotyl, roots, root tips, flowers, seeds, petioles, tubers, eyes, or stems of a potato plant as source material. Still further, the present disclosure provides methods that utilize any product produced from a potato plant as source material. In some aspects, the food product is a French fry, potato chip, dehydrated potato material, potato flakes, or potato granules.

In a particular aspect, four different potato varieties (Russet Burbank, Ranger Russet, Atlantic, and Snowden) were transformed with the pSIM1278 construct. In some embodiments, these potatoes are termed “GEN1” or Generation 1 or First Generation and include: E12 (Russet Burbank), J3 (Atlantic), J55 (Atlantic), F10 (Ranger Russet), and V11 (Snowden).

In another aspect, the Russet Burbank, Ranger Russet, and Atlantic potato variety was transformed with both the pSIM1278 and pSIM1678 constructs. In some embodiments, these potatoes are termed “GEN2” or Generation 2 or Second Generation and include: W8 (Russet Burbank), X17 (Ranger Russet), and Y9 (Atlantic).

Eight events were identified that exhibited low acrylamide content and/or increased black spot bruise tolerance: E12 (Russet Burbank), J3 (Atlantic), J55 (Atlantic), F10 (Ranger Russet), W8 (Russet Burbank), V11 (Snowden), X17 (Ranger Russet), and Y9 (Atlantic). The W8, X17, and Y9 events also exhibit increased resistance to late blight. All of these events are able to be detected by the event-specific, or construct-specific, detection methods disclosed herein.

One embodiment of this disclosure teaches construct-specific and variety/event-specific primers and probes and qPCR conditions to genetically identify each transformation event.

In one aspect, the present disclosure teaches qPCR methods utilized to identify a transformation event selected from the group consisting of: E12 (Russet Burbank), J3 (Atlantic), J55 (Atlantic), F10 (Ranger Russet), W8 (Russet Burbank), V11 (Snowden), X17 (Ranger Russet), and Y9 (Atlantic).

One embodiment of this disclosure teaches a method to examine a sample for the presence or absence of material derived from one or more transgenic plant events, comprising the steps of: (a) detecting the presence or absence in the sample of nucleic acids comprising one, more than one, or all of the nucleotide sequences having SEQ ID NOs 1-48; and (b) concluding based upon the presence or absence in the sample of said SEQ ID Nos, whether or not the sample contained genetic material from a plant transformation event. These sequences are indicative of a non-naturally occurring nucleotide junction that results from the transformation event. These non-natural junction sequences are outlined, inter alia, in Table 6, along with an indication of whether or not the junction sequence is contained on the “left” or “right” of the genetic insert. Further, a visual depiction of said events can be found, inter alia, in FIG. 5 and FIG. 6.

In some aspects, the presence or absence of nucleic acids in a sample is detected using PCR amplification. In some aspects, real-time PCR amplification is used. In some aspects, the taught methods utilize primer/probe sets from Table 7 having SEQ ID NOs: 52-90, or variants of said primer/probe sets. In some aspects, SEQ ID NOs: 49-51 are used to detect a control.

In some embodiments, the sample comprises the insert region of pSIM1278 that is present in event E12. In a further embodiment, event E12 contains inverted repeats of potato DNA effective for inhibition of expression of the endogenous asparagine synthetase-1 gene and the endogenous polyphenol oxidase-5 gene, in addition to inverted repeats of the endogenous potato promoters for the phosphorylase-L and dikinase R1 genes.

In some embodiments, the sample comprises the insert region of pSIM1278 that is present in event F10. In a further embodiment, event F10 contains inverted repeats of potato DNA effective for inhibition of expression of the endogenous asparagine synthetase-1 gene and the endogenous polyphenol oxidase-5 gene, in addition to inverted repeats of the endogenous potato promoters for the phosphorylase-L and dikinase R1 genes.

In some embodiments, the sample comprises the insert region of pSIM1278 that is present in event J3. In a further embodiment, event J3 contains inverted repeats of potato DNA effective for inhibition of expression of the endogenous asparagine synthetase-1 gene and the endogenous polyphenol oxidase-5 gene, in addition to inverted repeats of the endogenous potato promoters for the phosphorylase-L and dikinase R1 genes.

In some embodiments, the sample comprises the insert region of pSIM1278 that is present in event J55. In a further embodiment, event J55 contains inverted repeats of potato DNA effective for inhibition of expression of the endogenous asparagine synthetase-1 gene and the endogenous polyphenol oxidase-5 gene, in addition to inverted repeats of the endogenous potato promoters for the phosphorylase-L and dikinase R1 genes.

In some embodiments, the sample comprises the insert region of pSIM1278 that is present in event V11. In a further embodiment, event V11 contains inverted repeats of potato DNA effective for inhibition of expression of the endogenous asparagine synthetase-1 gene and the endogenous polyphenol oxidase-5 gene, in addition to inverted repeats of the endogenous potato promoters for the phosphorylase-L and dikinase R1 genes.

In some embodiments, the sample comprises the insert region of pSIM1278 and the insert region of pSIM1678 that are present in event W8. In a further embodiment, event W8 contains inverted repeats of potato DNA effective for inhibition of expression of the endogenous asparagine synthetase-1 gene and the endogenous polyphenol oxidase-5 gene, in addition to inverted repeats of the endogenous potato promoters for the phosphorylase-L and dikinase R1 genes. In a further embodiment, event W8 contains inverted repeats of potato DNA effective for inhibition of expression of the endogenous vacuolar acid invertase gene, in addition to sense potato DNA effective for expression of the late blight resistance gene Rpi-Vnt1.

In some embodiments, the sample comprises the insert region of pSIM1278 and the insert region of pSIM1678 that are present in event X17. In a further embodiment, event X17 contains inverted repeats of potato DNA effective for inhibition of expression of the endogenous asparagine synthetase-1 gene and the endogenous polyphenol oxidase-5 gene, in addition to inverted repeats of the endogenous potato promoters for the phosphorylase-L and dikinase R1 genes. In a further embodiment, event X17 contains inverted repeats of potato DNA effective for inhibition of expression of the endogenous vacuolar acid invertase gene, in addition to sense potato DNA effective for expression of the late blight resistance gene Rpi-Vnt1.

In some embodiments, the sample comprises the insert region of pSIM1278 and the insert region of pSIM1678 that are present in event Y9. In a further embodiment, event Y9 contains inverted repeats of potato DNA effective for inhibition of expression of the endogenous asparagine synthetase-1 gene and the endogenous polyphenol oxidase-5 gene, in addition to inverted repeats of the endogenous potato promoters for the phosphorylase-L and dikinase R1 genes. In a further embodiment, event Y9 contains inverted repeats of potato DNA effective for inhibition of expression of the endogenous vacuolar acid invertase gene, in addition to sense potato DNA effective for expression of the late blight resistance gene Rpi-Vnt1.

In one embodiment, the disclosure provides qPCR protocols utilizing a nucleotide probe labelled at the 5′ end with 6-carboxyfluorescein and at the 3′ end with Black Hole Quenchers™ However, it will be understood by a skilled artisan that any hybridization probe and any reporter molecule may be constructed.

In some embodiments, the efficiency of the PCR amplification is 90% to 110%. In another embodiment, the linearity of the PCR amplification is measured by the R² value. In some embodiments, the R² value is greater than or equal to 0.98. In some embodiments, the PCR amplification can detect at least 24 pg of potato leaf DNA between 34 and 35 cycles of amplification using an annealing/extension temperature of 60° C. In some embodiments, the PCR amplification is robust. In some embodiments, the PCR amplification has thermal cycling conditions comprising: (a) One PCR cycle at 95° C. for 600 sec for an initial denaturation; (b) Forty-five PCR cycles: at 95° C. for 15 sec for denaturation and then 60° C. for 60 sec for annealing/extension. In some embodiments, the thermal cycling conditions comprise: (a) One PCR cycle at 95° C. for 600 sec for an initial denaturation; (b) Forty PCR cycles: at 95° for 15 sec for denaturation and then 60° C. for 15 sec for annealing and then 72° C. for 10 sec for extension.

In some embodiments, the sample comprises plants or parts thereof, including flowers, tepals, petals, sepals, anthers, pollen, seeds, leaves, petioles, stems, roots, rhizomes, stolons, tubers or shoots, or portions thereof, plant cells, plant protoplasts and/or plant tissues, and/or plant-derived material, preferably food or feed material, including processed food or feed material. In some embodiments, the processed food is selected from the group consisting of French fries, potato chips, dehydrated potato material, potato flakes, potato protein, potato flour, and potato granules.

In some embodiments, the sample comprises a food product. In further embodiments, the food product comprises a mix of material derived from events E12, F10, J3, J55, V11, W8, X17, and/or Y9. In some embodiments, the material comprises potato fry, potato chip, potato flake, and potato tuber.

In some embodiments, the potato tuber and/or fry derived from events E12, F10, J3, J55, V11, W8, X17, and/or Y9 comprises about less than 1% of the total food product. In some embodiments, the potato tuber and/or fry derived from events E12, F10, J3, J55, V11, W8, X17, and/or Y9 comprises about less than 0.5% of the total food product. In some embodiments, the potato tuber and/or fry derived from events E12, F10, J3, J55, V11, W8, X17, and/or Y9 comprises about 0.2% of the total food product.

In some embodiments, the potato chip derived from events E12, F10, J3, J55, V11, W8, X17, and/or Y9 comprises about less than 10% of the total food product. In some embodiments, the potato chip derived from events E12, F10, J3, J55, V11, W8, X17, and/or Y9 comprises about 5% of the total food product.

In some embodiments, the potato flake derived from events E12, F10, J3, J55, V11, W8, X17, and/or Y9 comprises about less than 15% of the total food product. In some embodiments, the potato flake derived from events E12, F10, J3, J55, V11, W8, X17, and/or Y9 comprises about less than 8% of the total food product. In some embodiments, the potato flake derived from events E12, F10, J3, J55, V11, W8, X17, and/or Y9 comprises about 2.5% of the total food product.

Taught herein is an isolated nucleotide sequence comprising a sequence selected from SEQ ID NOs 1-90.

Taught herein is a kit for examining a sample for the potential presence or absence of material derived from one or more transformation plant events, the kit comprising one, more than one, or all primer/probe sets, or variants of said primer/probe sets. Kits of the present disclosure may also include directions/instructions for use of said kit.

In addition to the exemplary aspects and embodiments described above, further aspects and embodiments will become apparent by study of the following descriptions.

### BRIEF DESCRIPTION OF THE DRAWINGS

FIG. 1 depicts the pSIM1278 transformation vector. The vector backbone region, on the left, is 9,512 bp long, as it starts at position 10,149 bp and ends at position 19,660 bp. The backbone DNA consists mainly of bacterial DNA which provides support maintenance of the DNA insert prior to plant transformation. The DNA insert region (right side), including flanking Border sequences, is 10,148 bp long (from 1 bp to 10,148 bp). The DNA insert was stably integrated into the potato genome upon transformation.

FIG. 2 provides a schematic representation of the two silencing cassettes in the DNA insert inserted in the pSIM1278 transformation vector. Each silencing cassette contains two copies of two gene fragments separated by a spacer. Two copies of a DNA segment comprising fragments of four targeted genes, namely Asn-1, Ppo-5, Ph1 and R1, were inserted as inverted repeats between two convergent promoters, indicated as Pro, that are predominantly active in tubers. Plants containing the resulting silencing cassette produce a diverse and unpolyadenylated array of RNA molecules in tubers that dynamically and vigorously silence the intended target genes. The size of the RNA molecules was generally smaller than the distance between the two promoters employed because convergent transcription results in collisional transcription.

FIG. 3 depicts the pSIM1678 transformation vector of the present invention. The vector backbone region, on the left, is 9,512 bp long, as it starts at position 9,091 bp and ends at position 18,602 bp. The backbone DNA consists mainly of bacterial DNA which provides support maintenance of the DNA insert prior to plant transformation. The DNA insert region (right side), including flanking Border sequences, is 9,090 bp long (from 1 bp to 9,090 bp). The DNA insert was stably integrated into the potato genome upon transformation.

FIG. 4A-D shows a diagram of the structures of DNA inserts in potato events E12, F10, J3, and J55. Abbreviations are as follows: LB=Left Border (a 25-base pair sequence) similar to A. tumefaciens T-DNA border, AGP=the promoter of the ADP glucose pyrophosphorylase gene, GBS=the promoter of the granule-bound starch synthase gene, RB=Right Border (a 25-base pair sequence) similar to A. tumefaciens T-DNA border, ASN1=probe used in DNA gel blot hybridization and derived from the asparagine synthase 1 (Asn1) gene, fASN1=fragment of the asparagine synthase 1 (Asn1) gene, fPPO5=fragment of the polyphenol oxidase 5 (Ppo 5) gene, pPHL=fragment of the promoter of the Phosphorylase-L gene used in the second inverted repeat cassette, PHL=probe used in DNA gel blot hybridization and derived from the promoter of the Phosphorylase-L gene, pRL=fragment of the promoter of the water dikinase R1 gene used in the second inverted repeat cassette, spacer=the sequence between the arms of each inverted repeat, RV=Restriction enzyme EcoRV, Hd=Restriction enzyme Hind III, R1=Restriction enzyme EcoRI, Sc=Restriction enzyme ScaI. Heavy black lines denote probes to various regions of the DNA insert used in DNA gel blot hybridization. Bent arrows denote transcription start site for each respective promoter. White arrowheads depict the direction of each strand (sense or antisense) for a given gene or promoter fragment in each inverted repeat cassette. The numbers depict the nucleotide position in the DNA insert. Nucleotide position 1 is the start of the AGP promoter after the LB. Table 2 gives further details on each element of the DNA insert. The cultivars are depicted as follows: FIG. 4A—F10 Insert; FIG. 4B—E12 Insert; FIG. 4C—J3 Insert; FIG. 4D—J55 Insert.

FIG. 5 shows construct-specific junctions in the insert regions of plasmid constructs pSIM1278 and pSIM1678. The numbers below the insert regions indicate construct-specific junctions and correspond to SEQ ID NOs: 3-16 and SEQ ID NOs: 42-48 of Table 6. Abbreviations are as follows for the insert region of pSIM1278: LB=Left Border (a 25-base pair sequence) similar to A. tumefaciens T-DNA border, pAGP=the promoter of the ADP glucose pyrophosphorylase gene, pGbss=the promoter of the granule-bound starch synthase gene, RB=Right Border (a 25-base pair sequence) similar to A. tumefaciens T-DNA border, ASN=fragment of the asparagine synthase 1 (Asn1) gene, PPO=fragment of the polyphenol oxidase 5 (Ppo 5) gene, PHL=fragment of the promoter of the Phosphorylase-L gene used in the second inverted repeat cassette, RL=fragment of the promoter of the water dikinase R1 gene used in the second inverted repeat cassette, red box=the spacer sequence between the arms of each inverted repeat. Abbreviations are as follows for the insert region of pSIM1678: LB=Left Border (a 25-base pair sequence) similar to A. tumefaciens T-DNA border, pAGP=the promoter of the ADP glucose pyrophosphorylase gene, pGbss=the promoter of the granule-bound starch synthase gene, RB=Right Border (a 25-base pair sequence) similar to A. tumefaciens T-DNA border, pVnt1=the native promoter of the late blight resistance gene Rpi-vnt1, Vnt1=gene that confers resistance to late blight (Phytophthora infestans), tVnt1=the native terminator of the late blight resistance gene Rpi-vnt1, Inv=fragment of the vacuolar acid invertase gene. Arrows depict the direction of each strand (sense or antisense) for a given gene or promoter fragment in each inverted repeat cassette or the direction of transcription for a given promoter. Tables 2 and 4 give further details on each element of each insert region.

FIG. 6 A-I shows event-specific junctions and construct-specific junctions for Innate™ 1.0 Inserts (pSIM1278) Cultivars E12, F10, V11, J3, J55, and E56 and Innate™ 2.0 Inserts (pSIM1278 and pSIM1678) Cultivars W8, X17, and Y9. The numbers below the insert regions indicate construct-specific junctions and correspond to SEQ ID NOs found in Table 6. The numbers above the insert regions indicate event-specific junctions and correspond to SEQ ID NOs found in Table 6. Abbreviations are as described in FIG. 5. The cultivar inserts are depicted as follows: FIG. 6A—E12 Structure; FIG. 6B—F10 Structure; FIG. 6C V11 Structure; FIG. 6D—J3 Structure; FIG. 6E—J55 Structure; FIG. 6F—E56 Structure; FIG. 6G W8 Structure; FIG. 6H—X17 Structure; FIG. 6I—Y9 Structure.

FIG. 7 illustrates that all of the DNA isolations performed on food mixes from Ranger Russet event F10 tuber, fry, and flake and from Atlantic event J3 tuber and chip were able to be amplified at the lowest percentage of ground Innate™ food products mixed into commercial variety food products. There was one false negative in F10 flake at 2.5%. These results demonstrate that the disclosed method produces DNA of sufficient quality to be used in qPCR testing. Innate™ is a trademark utilized by J. R. Simplot to indicate potato plants that have been transformed with the pSIM1278 and/or pSIM1678 transformation vector and food products made from said plants.

FIG. 8 illustrates the process for constructing plasmid pSIM1278, utilizing the DNA sequences as described in Table 1 and Table 2. The starting vector, pCAMBIA1301, contains the origins of replications in the final pSIM1278 backbone.

FIG. 9 illustrates the construction of T-DNA expression cassettes in pSIM1278. Fusion PCR was used to amplify elements 1A (pAgp—1st copy), 1B (pAgp—2nd copy), 2 (Asn1, Ppo5), 3 (Ppo5, Asn1), 4 (pGbss—1st copy) and 7 (Spacer1, Ppo5, Asn1). Elements 5 (PhL, R1) and 6 (Spacer2, R1, PhL, pGbss) were synthesized by the Blue Heron Biotechnology, Inc. (Bothell, Wash.) based on the sequence from the potato genome. Elements 8, 9, and 10 were generated by ligating building blocks shown in the figure. In the end, three fragments, 10, 11 and 6 were created to span the desired expression cassette. These three fragments were ligated and inserted into the KpnI-SacI restriction sites shown in FIG. 8 to generate pSIM1278.

FIG. 10 illustrates the process for constructing plasmid pSIM1678, utilizing the DNA sequences as described in Table 3 and Table 4. The starting vector, pSIM1278, contains the final pSIM1678 backbone. One of skill in the art would be able to utilize the Examples and FIG. 9 and FIG. 10 to transform any potato plant, which would then contain non-naturally occurring nucleotide junctions detectable according to the methods taught herein.

### DETAILED DESCRIPTION

### Definitions

In the description and tables which follow, a number of terms are used. In order to provide a clear and consistent understanding of the specification and claims, including the scope to be given such terms, the following definitions are provided.

The term “a” or “an” refers to one or more of that entity; for example, “a primer” refers to one or more primers or at least one primer. As such, the terms “a” (or “an”), “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein, the term “allele” is any of one or more alternative forms of a gene which relate to one trait or characteristic. In a diploid cell or organism, the two alleles of a given gene occupy corresponding loci on a pair of homologous chromosomes.

As used herein, the term “amino acid sequence” includes an oligopeptide, peptide, polypeptide, or protein and fragments thereof that are isolated from, native to, or naturally occurring in a plant, or are synthetically made but comprise the nucleic acid sequence of the endogenous counterpart.

As used herein, the term “artificially manipulated” means to move, arrange, operate or control by the hands or by mechanical means or recombinant means, such as by genetic engineering techniques, a plant or plant cell, so as to produce a plant or plant cell that has a different biological, biochemical, morphological, or physiological phenotype and/or genotype in comparison to unmanipulated, naturally-occurring counterpart.

As used herein, the term “asexual propagation” means producing progeny by generating an entire plant from leaf cuttings, stem cuttings, root cuttings, tuber eyes, stolons, single plant cells protoplasts, callus and the like, that does not involve fusion of gametes.

As used herein, the term “backbone” means a nucleic acid sequence of a binary vector that excludes the DNA insert sequence intended for transfer.

As used herein, the term “backcrossing” is a process in which a breeder repeatedly crosses hybrid progeny back to one of the parents, for example, a first generation hybrid F₁ with one of the parental genotypes of the F₁ hybrid.

As used herein, the term “black spot bruise” describes a condition wherein black spots found in bruised tuber tissue are a result of a pigment called melanin that is produced following the injury of cells and gives tissue a brown, gray or black appearance. Melanin is formed when phenol substrates and an appropriate enzyme come in contact with each other as a result of cellular damage. The damage does not require broken cells. However, mixing of the substrate and enzyme must occur, usually when the tissue is impacted. Black spots occur primarily in the perimedullary tissue just beneath the vascular ring, but may be large enough to include a portion of the cortical tissue.

As used herein, the term “border-like sequences” means the following. A “border-like” sequence is isolated from the selected plant species that is to be modified, or from a plant that is sexually-compatible with the plant species to be modified, and functions like the border sequences of Agrobacterium. That is, a border-like sequence of the present disclosure promotes and facilitates the integration of a polynucleotide to which it is linked. A DNA insert of the present disclosure preferably contains border-like sequences. A border-like sequence of a DNA insert is between 5-100 bp in length, 10-80 bp in length, 15-75 bp in length, 15-60 bp in length, 15-50 bp in length, 15-40 bp in length, 15-30 bp in length, 16-30 bp in length, 20-30 bp in length, 21-30 bp in length, 22-30 bp in length, 23-30 bp in length, 24-30 bp in length, 25-30 bp in length, or 26-30 bp in length. A DNA insert left and right border sequences can be isolated from and/or native to the genome of a plant that is to be modified. A DNA insert border-like sequence is not identical in nucleotide sequence to any known Agrobacterium-derived T-DNA border sequence. Thus, a DNA insert border-like sequence may possess 1, 2, 3, 4, 5, 6, 7, 8, 9, 10, 11, 12, 13, 14, 15, 16, 17, 18, 19, 20, or more nucleotides that are different from a T-DNA border sequence from an Agrobacterium species, such as Agrobacterium tumefaciens or Agrobacterium rhizogenes. That is, a DNA insert border, or a border-like sequence of the present disclosure has at least 95%, at least 90%, at least 80%, at least 75%, at least 70%, at least 60% or at least 50% sequence identity with a T-DNA border sequence from an Agrobacterium species, such as Agrobacterium tumefaciens or Agrobacterium rhizogenes, but not 100% sequence identity. As used herein, the descriptive terms “DNA insert border” and “DNA insert border-like” are exchangeable. A border-like sequence can be isolated from a plant genome and be modified or mutated to change the efficiency by which it is capable of integrating a nucleotide sequence into another nucleotide sequence. Other polynucleotide sequences may be added to or incorporated within a border-like sequence of the present disclosure. Thus, a DNA insert left border or a DNA insert right border may be modified so as to possess 5′- and 3′-multiple cloning sites, or additional restriction sites. A DNA insert border sequence may be modified to increase the likelihood that backbone DNA from the accompanying vector is not integrated into the plant genome.

As used herein, the term “chip” is a thin slice of potato that has been deep fried or baked until crunchy. Potato chips are commonly served as an appetizer, side dish, or snack. Chips are also known as crisps.

As used herein, the verb “comprise” as is used in this description and in the claims and its conjugations are used in its non-limiting sense to mean that items following the word are included, but items not specifically mentioned are not excluded.

As used herein, a composition “consisting essentially of” certain elements is limited to the inclusion of those elements, as well as to those elements that do not materially affect the basic and novel characteristics of the inventive composition. Thus, so long as the composition does not affect the basic and novel characteristics of the instant disclosure, that is, does not contain foreign DNA that is not from the selected plant species or a plant that is sexually compatible with the selected plant species, then that composition may be considered a component of an inventive composition that is characterized by “consisting essentially of” language.

As used herein, the term “cotyledon” is a type of seed leaf. The cotyledon contains the food storage tissues of the seed.

As used herein, the term “degenerate primer” is an oligonucleotide that contains sufficient nucleotide variations that it can accommodate base mismatches when hybridized to sequences of similar, but not exact, homology.

As used herein, the term “dicotyledon” or “dicot” is a flowering plant whose embryos have two seed leaves or cotyledons. Examples of dicots include, but are not limited to, tobacco, tomato, potato, sweet potato, cassava, legumes including alfalfa and soybean, carrot, strawberry, lettuce, oak, maple, walnut, rose, mint, squash, daisy, and cactus.

As used herein, the term “DNA insert” according to the present disclosure means the DNA insert to be inserted into the genome of a plant comprises polynucleotide sequences native to that plant or has native genetic elements to that plant. In one example, for instance, the DNA insert of the potato variety J3 of the present disclosure is a 10,147 bp non-coding polynucleotide that is native to potato or wild potato, or a potato sexually-compatible plant, that is stably integrated into the genome of the plant cells upon transformation and silences genes involved in the expression of black spot bruises, asparagine accumulation, and senescence sweetening. The DNA insert preferably comprises two expression cassettes and is inserted into a transformation vector referred to as the pSIM1278 transformation vector. The first cassette comprises fragments of both the asparagine synthetase-1 gene (Asn1) and the polyphenol oxidase-5 gene (Ppo5), arranged as inverted repeats between the Agp promoter of the ADP glucose pyrophosphorylase gene (Agp) and the Gbss promoter of the granule-bound starch synthase gene (Gbss). These promoters are predominantly active in tubers. The function of the second cassette is to silence the promoters of the starch associated gene dikinase-R1 (R1) and the phosphorylase-L gene (PhL). This cassette is comprised of fragments of the promoters of the starch associated gene dikinase-R1 (R1) and the phosphorylase-L gene (PhL), operably linked to the same Agp and Gbss promoters as the first cassette. These expression cassettes contain DNA only from either the selected plant species or from a plant that is sexually compatible with the selected plant species.

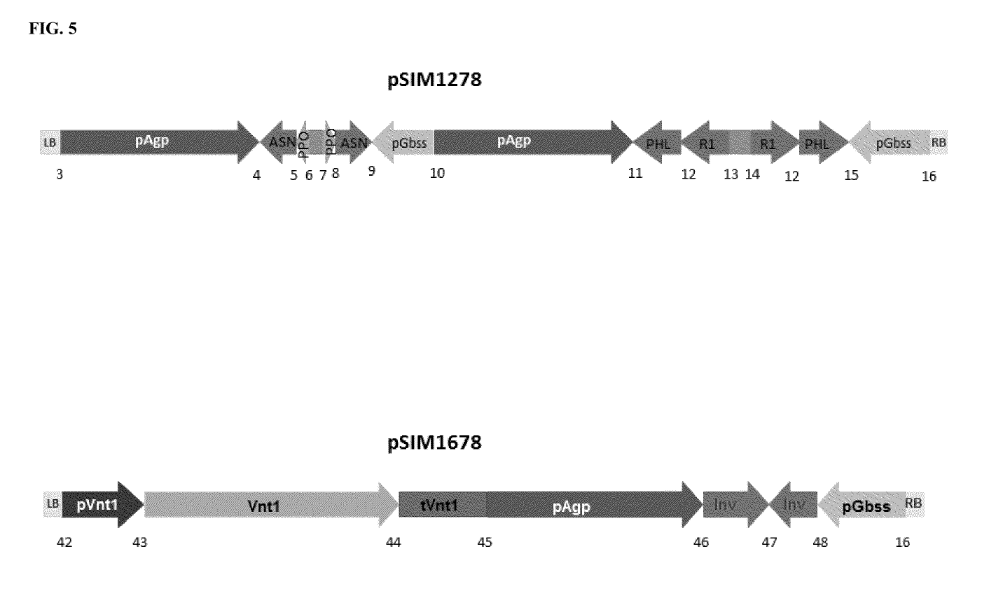

As used herein the term “non-natural nucleotide junction” or “non-naturally occurring nucleotide junction” refers to a sequence of nucleotides that do not occur in nature. Rather, these sequences are formed via a genetic transformation event. As aforementioned, the genetic transformation events described herein are created with expression cassettes that contain no non-native potato DNA. Thus, these non-natural nucleotide junctions are composed of potato nucleotides, but these nucleotides are in a genetic arrangement that does not occur in nature, but which results from the manipulation of man that occurs during the genetic transformation of the potato. Table 6 describes embodiments of these junction sequences. For event-specific junction sequences, Table 6 illustrates that: on one side of the junction is found nucleotides from the potato that has been transformed, and on the other side of the junction is found nucleotides that have been inserted via the transformation event. Thus, for event-specific junction sequences, the non-natural nucleotide junction represents the border where the inserted nucleotides meet the potato plant's native nucleotides. Also illustrated in Table 6 are construct junctions. These unique junction sequences occur in all of the transformation events and are not specific to a particular event, but rather will be present in any event that utilized the pSIM1278 construct and/or the pSIM1678 construct to perform transformation. These junctions represent the sequences of various genetic elements contained within the construct, for example the junction of where the ASN and PPO (ASN/PPO) elements come together. These construct-specific junctions are easily visualized by reference to FIG. 5.

As used herein, the term “efficiency” refers to a hallmark of Real-Time PCR assays. An ideal qPCR (quantitative PCR) reaction has an efficiency of 100% with a slope of −3.32, which correlates with a perfect doubling of PCR product during each cycle. However, slopes between −3.1 and −3.6 with efficiencies between 90 and 110% are generally considered acceptable (Commission, C. A. (2009). Definition of Minimum Performance Requirements for Analytical Methods of GMO Testing European Network of GMO Laboratories (ENGL), (October 2008), 1-8). Efficiency is established by replicated standard curves. Amplification efficiency is determined from the slope of the log-linear portion of the standard curve and is calculated as E=(10^{(-1/slope)}−1)*100. (Bustin, S. A., et al. (2009). The MIQE Guidelines: Minimum Information for Publication of Quantitative Real-Time PCR Experiments. Clinical Chemistry, 55(4), 1-12. doi:10.1373/clinchem.2008.112797).

As used herein, the term “embryo” is the immature plant contained within a mature seed.

As used herein the term “event” refers to the unique DNA recombination event that took place in one plant cell, which was then used to generate entire transgenic plants. Plant cells are transformed with a binary transformation vector carrying a DNA insert of interest. Transformed cells are regenerated into transgenic plants, and each resulting transgenic plant represents a unique event. Molecular techniques such as Southern blot hybridization or PCR are used to confirm each transformed event. Each derived event is identified by an abbreviation (e.g. J3). Different events possess differences in the number of copies of DNA insert in the cell genome, the arrangement of the DNA insert copies and/or the DNA insert location in the genome. The events that result in optimal expression of genes in the DNA insert and exhibition of traits may be analyzed and studied further.

As used herein, the term “foreign,” with respect to a nucleic acid, means that the nucleic acid is derived from non-plant organisms, or derived from a plant that is not the same species as the plant to be transformed, or is derived from a plant that is not interfertile with the plant to be transformed, or does not belong to the species of the target plant. According to the present disclosure, foreign DNA or RNA represents nucleic acids that are naturally occurring in the genetic makeup of fungi, bacteria, viruses, mammals, fish or birds, but are not naturally occurring in the plant that is to be transformed. Thus, a foreign nucleic acid is one that encodes, for instance, a polypeptide that is not naturally produced by the transformed plant. A foreign nucleic acid does not have to encode a protein product. According to the present disclosure, a desired intragenic plant is one that does not contain any foreign nucleic acids integrated into its genome.

As used herein, the term “flake” refers to potato flakes which are created through an industrial process of cooking, mashing and dehydrating to yield a packaged convenience food that can be reconstituted by adding hot water or milk, producing a close approximation of mashed potatoes.

As used herein, the term “fry” is a baton of deep-fried potato. Fries are elongated pieces of fried potato that are served hot, either soft or crispy, and generally eaten as an accompaniment with lunch or dinner, or eaten as a snack.

As used herein, the term “gene” refers to the coding region and does not include nucleotide sequences that are 5′- or 3′- to that region. A functional gene is the coding region operably linked to a promoter or terminator. A gene can be introduced into a genome of a species, whether from a different species or from the same species, using transformation or various breeding methods.

As used herein, the term “gene converted” or “conversion” refers to plants which are developed by a plant breeding technique called backcrossing wherein essentially all of the desired morphological and physiological characteristics of a variety are recovered in addition to the one or more genes transferred into the variety via the backcrossing technique, via genetic engineering or via mutation. One or more loci may also be transferred.

As used herein, the term “genetic rearrangement” refers to the re-association of genetic elements that can occur spontaneously in vivo as well as in vitro which introduces a new organization of genetic material. For instance, the splicing together of polynucleotides at different chromosomal loci, can occur spontaneously in vivo during both plant development and sexual recombination. Accordingly, recombination of genetic elements by non-natural genetic modification techniques in vitro is akin to recombination events that also can occur through sexual recombination in vivo.

As used herein, the term “hypocotyl” is the portion of an embryo or seedling between the cotyledons and the root. Therefore, it can be considered a transition zone between shoot and root.

As used herein, the term “in frame” means the following. Nucleotide triplets (codons) are translated into a nascent amino acid sequence of the desired recombinant protein in a plant cell. Specifically, the present disclosure contemplates a first nucleic acid linked in reading frame to a second nucleic acid, wherein the first nucleotide sequence is a gene and the second nucleotide is a promoter or similar regulatory element.

As used herein, the term “integrate” refers to the insertion of a nucleic acid sequence from a selected plant species, or from a plant that is from the same species as the selected plant, or from a plant that is sexually compatible with the selected plant species, into the genome of a cell of a selected plant species. “Integration” refers to the incorporation of only native genetic elements into a plant cell genome. In order to integrate a native genetic element, such as by homologous recombination, the present disclosure may “use” non-native DNA as a step in such a process. Thus, the present disclosure distinguishes between the “use of” a particular DNA molecule and the “integration” of a particular DNA molecule into a plant cell genome.

As used herein, the term “introduction” refers to the insertion of a nucleic acid sequence into a cell, by methods including infection, transfection, transformation or transduction.

As used herein, the term “isolated” refers to any nucleic acid or compound that is physically separated from its normal, native environment. The isolated material may be maintained in a suitable solution containing, for instance, a solvent, a buffer, an ion, or other component, and may be in purified, or unpurified, form.

As used herein, the term “late blight” refers to a potato disease caused by the oomycete Phytophthora infestans and also known as ‘potato blight’ that can infect and destroy the leaves, stems, fruits, and tubers of potato plants.

As used herein, the term “leader” refers to a sequence that precedes (or is 5′ to) a gene and is transcribed but not translated.

As used herein, the term “level of detection” or “LOD” is the lowest amount or concentration of analyte in a sample, which can be reliably detected, but not necessarily quantified, as demonstrated by single-laboratory validation, according to the European Network of GMO Laboratories.

As used herein, the term “linearity” refers to a hallmark of optimized Real-Time PCR assays and is determined by the R² value obtained by linear regression analysis, which should be ≧0.98 (Bustin et al., 2009).

As used herein, the term “locus” confers one or more traits such as, for example, male sterility, herbicide tolerance, insect resistance, disease resistance, waxy starch, modified fatty acid metabolism, modified phytic acid metabolism, modified carbohydrate metabolism, and modified protein metabolism. The trait may be, for example, conferred by a naturally occurring gene introduced into the genome of the variety by backcrossing, a natural or induced mutation, or a transgene introduced through genetic transformation techniques. A locus may comprise one or more alleles integrated at a single chromosomal location.

As used herein, the term “marketable yield” is the weight of all tubers harvested that are between 2 and 4 inches in diameter. Marketable yield is measured in cwt (hundred weight) where cwt=100 pounds.

As used herein, the term “monocotyledon” or “monocot” is a flowering plant whose embryos have one cotyledon or seed leaf. Examples of monocots include, but are not limited to turf grass, maize, rice, oat, wheat, barley, sorghum, orchid, iris, lily, onion, and palm.

As used herein, the term “native” genetic element refers to a nucleic acid that naturally exists in, originates from, or belongs to the genome of a plant that is to be transformed. Thus, any nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species. In other words, a native genetic element represents all genetic material that is accessible to plant breeders for the improvement of plants through classical plant breeding. Any variants of a native nucleic acid also are considered “native” in accordance with the present disclosure. In this respect, a “native” nucleic acid may also be isolated from a plant or sexually compatible species thereof and modified or mutated so that the resultant variant is greater than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, or 60% similar in nucleotide sequence to the unmodified, native nucleic acid isolated from a plant. A native nucleic acid variant may also be less than about 60%, less than about 55%, or less than about 50% similar in nucleotide sequence. A “native” nucleic acid isolated from a plant may also encode a variant of the naturally occurring protein product transcribed and translated from that nucleic acid. Thus, a native nucleic acid may encode a protein that is greater than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, or 60% similar in amino acid sequence to the unmodified, native protein expressed in the plant from which the nucleic acid was isolated.

As used herein, the term “naturally occurring nucleic acid” is found within the genome of a selected plant species and may be a DNA molecule or an RNA molecule. The sequence of a restriction site that is normally present in the genome of a plant species can be engineered into an exogenous DNA molecule, such as a vector or oligonucleotide, even though that restriction site was not physically isolated from that genome. Thus, the present disclosure permits the synthetic creation of a nucleotide sequence, such as a restriction enzyme recognition sequence, so long as that sequence is naturally occurring in the genome of the selected plant species or in a plant that is sexually compatible with the selected plant species that is to be transformed.

As used herein, the term “operably linked” means combining two or more molecules in such a fashion that in combination they function properly in a plant cell. For instance, a promoter is operably linked to a structural gene when the promoter controls transcription of the structural gene.

As used herein, the term “plant” includes but is not limited to angiosperms and gymnosperms such as potato, tomato, tobacco, alfalfa, lettuce, carrot, strawberry, sugarbeet, cassava, sweet potato, soybean, maize, turf grass, wheat, rice, barley, sorghum, oat, oak, eucalyptus, walnut, and palm. Thus, a plant may be a monocot or a dicot. The word “plant,” as used herein, also encompasses plant cells, seed, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores. Plants may be at various stages of maturity and may be grown in liquid or solid culture, or in soil or suitable media in pots, greenhouses or fields. Expression of an introduced leader, trailer or gene sequences in plants may be transient or permanent. A “selected plant species” may be, but is not limited to, a species of any one of these “plants.”

As used herein, the term “plant parts” (or a potato plant, or a part thereof) includes but is not limited to protoplast, leaf, stem, root, root tip, anther, pistil, seed, embryo, pollen, ovule, cotyledon, hypocotyl, flower, tuber, eye, tissue, petiole, cell, meristematic cell, and the like.

As used herein, the term “plant species” is the group of plants belonging to various officially named plant species that display at least some sexual compatibility.

As used herein, the terms “plant transformation” and “cell culture” broadly refer to the process by which plant cells are genetically modified and transferred to an appropriate plant culture medium for maintenance, further growth, and/or further development.

As used herein, the term “precise breeding” refers to the improvement of plants by stable introduction of nucleic acids, such as native genes and regulatory elements isolated from the selected plant species, or from another plant in the same species as the selected plant, or from species that are sexually compatible with the selected plant species, into individual plant cells, and subsequent regeneration of these genetically modified plant cells into whole plants. Since no unknown or foreign nucleic acid is permanently incorporated into the plant genome, the inventive technology makes use of the same genetic material that is also accessible through conventional plant breeding.

As used herein, the term “primer” is an oligonucleotide that anneals to a nucleic acid sequence of interest. The primer serves as a starting point for nucleic acid synthesis. DNA polymerase, one enzyme that catalyzes this process, adds new nucleotides to the 3′ end of a DNA primer, and copies the opposite strand. For example, forward and reverse primers complementary to a DNA sequence of interest are used in a polymerase chain reaction (PCR) assay to amplify a DNA region of interest.

As used herein, the term “probe” is an oligonucleotide that has been labelled with a detectable molecule, such as a radioactive label, biotin, digoxygenin or fluorescein, and is complementary to a nucleic acid sequence of interest. For example, probes labeled at the 5′ end with 6-FAM (6-carboxyfluorescein) and at the 3′ end with a BHQ1 (Black Hole Quenchers™ 1) are used in Real-Time PCR for detection of nucleic acid sequences of interest. However, the term probe can also be used more generically, to refer to a nucleotide sequence that is capable of binding to a non-naturally occurring nucleotide junction sequence, irrespective of whether the probe has a r label attached thereon.

As used herein, the term “progeny” includes an F₁ potato plant produced from the cross of two potato plants and progeny further includes, but is not limited to, subsequent F₂, F₃, F₄, F₅, F₆, F₇, F₈, F₉, and F₁₀ generational crosses with the recurrent parental line.

As used herein, the term “Quantitative Trait Loci” (QTL) refers to genetic loci that control to some degree numerically representable traits that are usually continuously distributed.

As used herein, the term “recombinant” broadly describes various technologies whereby genes can be cloned, DNA can be sequenced, and protein products can be produced. As used herein, the term also describes proteins that have been produced following the transfer of genes into the cells of plant host systems.

As used herein, the term “regeneration” refers to the development of a plant from tissue culture.

As used herein, the term “regulatory sequences” refers to those sequences which are standard and known to those in the art, which may be included in the expression vectors to increase and/or maximize transcription of a gene of interest or translation of the resulting RNA in a plant system. These include, but are not limited to, promoters, peptide export signal sequences, introns, polyadenylation, and transcription termination sites. Methods of modifying nucleic acid constructs to increase expression levels in plants are also generally known in the art (see, e.g. Rogers et al., 260 J. Biol. Chem. 3731-38, 1985; Cornejo et al., 23 Plant Mol. Biol. 567: 81, 1993). In engineering a plant system to affect the rate of transcription of a protein, various factors known in the art, including regulatory sequences such as positively or negatively acting sequences, enhancers and silencers, as well as chromatin structure may have an impact. The present disclosure provides that at least one of these factors may be utilized in engineering plants to express a protein of interest. The regulatory sequences of the present disclosure are native genetic elements, i.e., are isolated from the selected plant species to be modified.

As used herein, the term “selectable marker” is typically a gene that codes for a protein that confers some kind of resistance to an antibiotic, herbicide or toxic compound, and is used to identify transformation events. Examples of selectable markers include the streptomycin phosphotransferase (spt) gene encoding streptomycin resistance, the phosphomannose isomerase (pmi) gene that converts mannose-6-phosphate into fructose-6 phosphate; the neomycin phosphotransferase (nptII) gene encoding kanamycin and geneticin resistance, the hygromycin phosphotransferase (hpt or aphiv) gene encoding resistance to hygromycin, acetolactate synthase (als) genes encoding resistance to sulfonylurea-type herbicides, genes coding for resistance to herbicides which act to inhibit the action of glutamine synthase such as phosphinothricin or basta (e.g., the bar gene), or other similar genes known in the art.

As used herein, the term “sense suppression” is a reduction in expression of an endogenous gene by expression of one or more an additional copies of all or part of that gene in transgenic plants.

As used herein, the term “specific gravity” is an expression of density and is a measurement of potato quality. There is a high correlation between the specific gravity of the tuber and the starch content and percentage of dry matter or total solids. A higher specific gravity contributes to higher recovery rate and better quality of the processed product.

The term “stringent conditions” used herein refers to conditions under which a specific hybrid is formed, but a non-specific hybrid is not formed, or is much less likely to form. For example, the stringent conditions may be conditions under which DNA (e.g. a probe) having high homology (90% or more, or 95% or more) with DNA of a non-naturally occurring nucleotide junction—e.g. a probe sequence having a sequence with high homology to a sequence of Table 6—hybridizes to said sequence. The stringent conditions may refer to conditions under which hybridization occurs at a temperature lower than the melting temperature (Tm) of a perfect hybrid by about 5° C. to about 30° C. (in aspects about 10° C. to about 25° C.). For example, the conditions described in J. Sambrook et al., Molecular Cloning, A Laboratory Manual, Second Edition, Cold Spring Harbor Laboratory Press (1989) (particularly the conditions described in §11.45 “Conditions for Hybridization of Oligonucleotide Probes”) may be used as the stringent conditions. Thus, an indication that two nucleic acid sequences are substantially homologous is that the two molecules hybridize to each other under stringent conditions. Stringent conditions are sequence dependent and are different under different environmental parameters. Generally, stringent conditions are selected to be about 5° C. to 20° C. lower than the thermal melting point (Tₘ) for the specific sequence at a defined ionic strength and pH. The Tₘ is defined as the temperature in degrees Celsius, at which 50% of all molecules of a given DNA sequence are hybridized into a double strand, and 50% are present as single strands.

As used herein, the term “T-DNA-like” sequence is a nucleic acid sequence that is isolated from a selected plant species, or from a plant that is sexually compatible with the selected plant species, and which shares at least 75%, 80%, 85%, 90%, or 95%, but not 100%, sequence identity with Agrobacterium species T-DNA. The T-DNA-like sequence may contain one or more border or border-like sequences that are each capable of integrating a nucleotide sequence into another polynucleotide.

As used herein, the term “total yield” refers to the total weight of all harvested tubers.

As used herein, the term “trailer” refers to transcribed but not translated sequence following (or 3′ to) a gene.

As used herein, the term “transcribed DNA” is DNA comprising both a gene and the untranslated leader and trailer sequences that are associated with that gene. The gene is transcribed as a single mRNA by the action of the preceding promoter.

As used herein, the term “transformation of plant cells” is a process by which DNA is stably integrated into the genome of a plant cell. “Stably” refers to the permanent, or non-transient retention and/or expression of a polynucleotide in and by a cell genome. Thus, a stably integrated polynucleotide is one that is a fixture within a transformed cell genome and can be replicated and propagated through successive progeny of the cell or resultant transformed plant. Transformation may occur under natural or artificial conditions using various methods well known in the art. Transformation may rely on any known method for the insertion of nucleic acid sequences into a prokaryotic or eukaryotic host cell, including Agrobacterium-mediated transformation protocols, viral infection, whiskers, electroporation, heat shock, lipofection, polyethylene glycol treatment, micro-injection, and particle bombardment.

As used herein, the term “transgene” is a gene that will be inserted into a host genome.

As used herein, the term “transgenic plant” is a genetically modified plant which contains at least one transgene.

As used herein, the term “tuber” refers to a type of modified plant structure that is enlarged to store nutrients. It is used by plants to survive the winter or dry months, to provide energy and nutrients for regrowth during the next growing season, and as a means of asexual reproduction. It can be derived from stems or roots. Potatoes are stem tubers.

As used herein, the term “variant” is understood to mean a nucleotide or amino acid sequence that deviates from the standard, or given, nucleotide or amino acid sequence of a particular gene or protein. The terms, “isoform,” “isotype,” and “analog” also refer to “variant” forms of a nucleotide or an amino acid sequence. An amino acid sequence that is altered by the addition, removal or substitution of one or more amino acids, or a change in nucleotide sequence, may be considered a “variant” sequence. The variant may have “conservative” changes, wherein a substituted amino acid has similar structural or chemical properties, e.g., replacement of leucine with isoleucine. A variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software.

As used herein, the term “vine maturity” refers to a plant's ability to continue to utilize carbohydrates and photosynthesize. Vine maturity is scored on a scale of 1 to 5 where 1=dead vines and 5=vines green, still flowering.

### Innate™ Technologies

The insertion of desirable traits into the genome of potato plants presents particular difficulties because potato is tetraploid, highly heterozygous and sensitive to in-breeding depression. It is therefore very difficult to efficiently develop transgenic potato plants that produce less acrylamide and less harmful Maillard-reaction products, including N-Nitroso-N-(3-keto-1,2-butanediol)-3′-nitrotyramine (Wang et al., Arch Toxicol 70: 10-5, 1995), 5-hydroxymethyl-2-furfural (Janzowski et al., Food Chem Toxicol 38: 801-9, 2000), and other Maillard reaction products with mutagenic properties (Shibamoto, Prog Clin Biol Res 304: 359-76, 1989), during processing using conventional breeding.

Several methods have been tested and research is ongoing to reduce acrylamide through process changes, reduction in dextrose, and additives such as asparaginase, citrate, and competing amino acids. The required capital expense to implement process changes throughout the potato industry would cost millions of dollars. In addition to the expense, these process changes have significant drawbacks including potentially negative flavors associated with additives such as asparaginase or citrate. Typically, fry manufacturers add dextrose during processing of French fries to develop the desired golden brown color, but dextrose also increases the formation of acrylamide through the Maillard reaction. Significant reductions in acrylamide occur by merely omitting dextrose from the process; however, the signature golden brown colors must then be developed some other way (such as though the addition of colors like annatto) The use of alternate colors, results in an absence of the typical flavors that develop through those browning reactions. Another challenge with the use of additives to reduce reactants like asparagine is moisture migration that occurs during frozen storage with the resulting return of asparagine to the surface and increased acrylamide. Finally, the blackening that occurs after potatoes are bruised affects quality and recovery in processing French fries and chips. Damaged and bruised potatoes must be trimmed or are rejected before processing, resulting in quality challenges or economic loss.

A description of Innate™ technologies outlines the plant biological systems all working together to create the plants. These include trait identification, design of vectors, incorporation of vectors into Agrobacterium, recipient potato variety selection, transforming plants, and confirmation that the new potatoes contain the expected DNA inserts. The Innate™ methods allow the insertion of non-coding DNA into potato to develop new potato events with desired traits that are not plant pests.

The “native technology” strategy of the present disclosure addresses the need of the potato industry to improve the agronomic characteristics and nutritional value of potatoes by reducing the expression of polyphenol oxidase-5 (Ppo5), which is responsible for black spot bruise, the expression of asparagine synthetase-1 (Asn1), which is responsible for the accumulation of asparagine, a precursor in acrylamide formation, and/or the expression of phosphorylase-L and dikinase-R1, which are enzymes associated with the accumulation of reducing sugars that normally react with amino acids, such as asparagine, and form toxic Maillard products, including acrylamide.

The partial or complete silencing of these genes in tubers decreases the potential to produce acrylamide. Use of the Innate™ technologies of the disclosure allows for the incorporation of desirable traits into the genome of commercially valuable potato plant varieties by transforming the potatoes only with “native” genetic material, that is genetic material obtained from potato plants or plants that are sexually-compatible with potato plants, that comprises non-coding regulatory regions, without the integration of any foreign genetic material into the plant's genome.

Desirable traits include high tolerance to impact-induced black spot bruise, reduced formation of the acrylamide precursor asparagine and reduced accumulation of reducing sugars, with consequent decrease in accumulation of toxic Maillard products, including acrylamide, improved quality and food color control. The incorporation of these desirable traits into existing potato varieties is impossible to achieve through traditional breeding because potato is tetraploid, highly heterozygous and sensitive to inbreeding depression.

The non-coding potato plant DNA insert sequences used in the present disclosure are native to the potato plant genome and do not contain any Agrobacterium DNA. The DNA insert preferably comprises two expression cassettes and is inserted into a transformation vector referred to as the pSIM1278 transformation vector (described in U.S. Pat. No. 8,754,303, “Potato Cultivar J3”; U.S. Pat. No. 8,710,311 “Potato Cultivar F10”; U.S. Pat. No. 8,889,963 “Potato Cultivar J55”; and U.S. patent application Ser. No. 14/072,487 “Potato Cultivar E12”; and U.S. Pat. No. 8,889,964 “Potato Cultivar W8,” which also has the pSIM1678 vector, each of these patents and applications are incorporated herein by reference in their entirety).

The first cassette comprises fragments of both the asparagine synthetase-1 gene (Asn1) and the polyphenol oxidase-5 gene (Ppo5), arranged as inverted repeats between the Agp promoter of the ADP glucose pyrophosphorylase gene (Agp) and the Gbss promoter of the granule-bound starch synthase gene (Gbss). These promoters are predominantly active in tubers.

The function of the second cassette is to silence the promoters of the starch associated gene dikinase-R1 (R1) and the phosphorylase-L gene (PhL). This cassette is comprised of fragments of the promoters of the starch associated gene dikinase-R1 (R1) and the phosphorylase-L gene (PhL), operably linked to the same Agp and Gbss promoters as the first cassette. These expression cassettes contain no foreign DNA, and consist of DNA only from either the selected plant species or from a plant that is sexually compatible with the selected plant species.

A second DNA insert comes from the transformation vector referred to as pSIM1678 (described in U.S. Pat. No. 8,889,964, “Potato Cultivar W8,” which is incorporated herein by reference in its entirety) that comprises the Rpi-vnt1 expression cassette and a silencing cassette for the plant vacuolar invertase gene, VInv. The Rpi-vnt1 gene cassette consists of the VNT1 protein coding region regulated by its native promoter and terminator sequences to confer broad resistance to late blight, whereas the silencing cassette consists of an inverted repeat of sequence from the potato VInv gene flanked by opposing plant promoters, pGbss and pAgp. The function of the first cassette is to confer resistance to late blight, while the function of the second cassette is to silence the vacuolar invertase gene, reducing glucose and fructose.

Targeted gene silencing with native DNA reduces the level of the RNA transcripts of the targeted genes in the tubers of the potato events. In general, the inserted DNA contains silencing cassettes that, when expressed, generate variably-sized and unprocessed transcripts. These transcripts trigger the degradation of mRNAs that would normally code for an enzyme, like asparagine synthetase. This results in much reduced levels of the targeted “silenced” enzymes.

Asn1 and Ppo5 gene silencing is sufficient to significantly reduce acrylamide formation by two to four fold without further inhibiting the starch associated genes kinase-R1 (R1) and phosphorylase-L (PhL).

Thus, the tubers of the potato events incorporate highly desirable traits, including a reduced ratio in free amide amino acids asparagine and glutamine, which is associated with reduced acrylamide formation upon frying or baking Specifically, the potato varieties of the present disclosure are characterized by two- to more than four-fold reduction in free-asparagine content. Furthermore, the potato varieties of the present disclosure display a delay in the degradation of starch into the reducing sugars glucose and fructose during storage. Impairment of starch-to-sugar conversion further reduces senescence sweetening and acrylamide formation and limits heat-induced browning. Further, events W8, X17, and Y9, also show a resistance to late blight, which is resultant from the additional utilization of the pSIM1678 vector, in addition to the pSIM1278 vector, that is present in events J3, F10, J55, and E12.

Potato varieties of the present disclosure are therefore extremely valuable in the potato industry and food market, as their tubers produce significantly less acrylamide upon heat processing and do not carry any potentially harmful foreign genes.

The research leading to potato varieties which combine the advantageous characteristics referred to above is largely empirical. This research requires large investments of time, labor, and money. The development of a potato cultivar can often take up to eight years or more from greenhouse to commercial usage. Breeding begins with careful selection of superior parents to incorporate the most important characteristics into the progeny. Since all desired traits usually do not appear with just one cross, breeding must be cumulative.

Present breeding techniques continue with the controlled pollination of parental clones. Typically, pollen is collected in gelatin capsules for later use in pollinating the female parents. Hybrid seeds are sown in greenhouses and tubers are harvested and retained from thousands of individual seedlings. The next year one to four tubers from each resulting seedling are planted in the field, where extreme caution is exercised to avoid the spread of virus and diseases. From this first-year seedling crop, several “seed” tubers from each hybrid individual which survived the selection process are retained for the next year's planting. After the second year, samples are taken for density measurements and fry tests to determine the suitability of the tubers for commercial usage. Plants which have survived the selection process to this point are then planted at an expanded volume the third year for a more comprehensive series of fry tests and density determinations. At the fourth-year stage of development, surviving selections are subjected to field trials in several states to determine their adaptability to different growing conditions. Eventually, the varieties having superior qualities are transferred to other farms and the seed increased to commercial scale. Generally, by this time, eight or more years of planting, harvesting and testing have been invested in attempting to develop the new and improved potato cultivars.

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Such foreign additional and/or modified genes are referred to herein collectively as “transgenes”. Over the last fifteen to twenty years several methods for producing transgenic plants have been developed, and the present disclosure, in particular embodiments, also relates to transformed versions of the claimed variety or line.

Plant transformation involves the construction of an expression vector which will function in plant cells. Such a vector comprises DNA comprising a gene under control of, or operatively linked to, a regulatory element (for example, a promoter). The expression vector may contain one or more such operably linked gene/regulatory element combinations. The vector(s) may be in the form of a plasmid, and can be used alone or in combination with other plasmids, to provide transformed potato plants, using transformation methods as described below to incorporate transgenes into the genetic material of the potato plant(s).

Traditional plant breeding typically relies on the random recombination of plant chromosomes to create varieties that have new and improved characteristics. According to standard, well-known techniques, genetic “expression cassettes,” comprising genes and regulatory elements, are inserted within the borders of Agrobacterium-isolated transfer DNAs (“T-DNAs”) and integrated into plant genomes. Agrobacterium-mediated transfer of T-DNA material typically comprises the following standard procedures: (1) in vitro recombination of genetic elements, at least one of which is of foreign origin, to produce an expression cassette for selection of transformation, (2) insertion of this expression cassette, often together with at least one other expression cassette containing foreign DNA, into a T-DNA region of a binary vector, which usually consists of several hundreds of basepairs of Agrobacterium DNA flanked by T-DNA border sequences, (3) transfer of the sequences located between the T-DNA borders, often accompanied with some or all of the additional binary vector sequences from Agrobacterium to the plant cell, and (4) selection of stably transformed plant cells that display a desired trait, such as an increase in yield, improved vigor, enhanced resistance to diseases and insects, or greater ability to survive under stress.

Thus, genetic engineering methods may rely on the introduction of foreign, not-endogenous nucleic acids, including regulatory elements such as promoters and terminators, and genes that are involved in the expression of a new trait or function as markers for identification and selection of transformants, from viruses, bacteria and plants. Marker genes are typically derived from bacterial sources and confer antibiotic or herbicide resistance. Classical breeding methods are laborious and time-consuming, and new varieties typically display only relatively modest improvements.

In the “anti-sense” technology, the sequence of native genes is inverted to silence the expression of the gene in transgenic plants.

### Expression Vectors for Potato Transformation: Marker Genes

Expression vectors include at least one genetic marker, operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene which, when under the control of plant regulatory signals, confers resistance to kanamycin. Fraley et al., Proc. Natl. Acad. Sci. U.S.A., 80:4803 (1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase and aminoglycoside-3′-adenyl transferase, the bleomycin resistance determinant. Hayford et al., Plant Physiol. 86:1216 (1988), Jones et al., Mol. Gen. Genet., 210:86 (1987), Svab et al., Plant Mol. Biol. 14:197 (1990) Hille et al., Plant Mol. Biol. 7:171 (1986). Other selectable marker genes confer resistance to herbicides such as glyphosate, glufosinate or bromoxynil. Comai et al., Nature 317:741-744 (1985), Gordon-Kamm et al., Plant Cell 2:603-618 (1990) and Stalker et al., Science 242:419-423 (1988).

Selectable marker genes for plant transformation not of bacterial origin include, for example, mouse dihydrofolate reductase, plant 5-enolpyruvylshikimate-3-phosphate synthase and plant acetolactate synthase. Eichholtz et al., Somatic Cell Mol. Genet. 13:67 (1987), Shah et al., Science 233:478 (1986), Charest et al., Plant Cell Rep. 8:643 (1990).

Another class of marker genes for plant transformation requires screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include .beta.-glucuronidase (GUS), .beta.-galactosidase, luciferase and chloramphenicol acetyltransferase. Jefferson, R. A., Plant Mol. Biol. Rep. 5:387 (1987), Teeri et al., EMBO J. 8:343 (1989), Koncz et al., Proc. Natl. Acad. Sci. USA 84:131 (1987), DeBlock et al., EMBO J. 3:1681 (1984).

In vivo methods for visualizing GUS activity that do not require destruction of plant tissue are available. Molecular Probes publication 2908, IMAGENE GREEN, p. 1-4 (1993) and Naleway et al., J. Cell Biol. 115:151a (1991). However, these in vivo methods for visualizing GUS activity have not proven useful for recovery of transformed cells because of low sensitivity, high fluorescent backgrounds and limitations associated with the use of luciferase genes as selectable markers.

In some aspects, a gene encoding Green Fluorescent Protein (GFP) has been utilized as a marker for gene expression in prokaryotic and eukaryotic cells. Chalfie et al., Science 263:802 (1994). GFP and mutants of GFP may be used as screenable markers.

### Expression Vectors for Potato Transformation: Promoters

Genes included in expression vectors must be driven by a nucleotide sequence comprising a regulatory element, for example, a promoter. Several types of promoters are well known in the transformation arts as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters that initiate transcription only in a certain tissue are referred to as “tissue-specific”. A “cell-type” specific promoter primarily drives expression in certain cell types in one or more organs, for example, vascular cells in roots or leaves. An “inducible” promoter is a promoter which is under environmental control. Examples of environmental conditions that may effect transcription by inducible promoters include anaerobic conditions or the presence of light. Tissue-specific, tissue-preferred, cell type specific, and inducible promoters constitute the class of “non-constitutive” promoters. A “constitutive” promoter is a promoter that is active under most environmental conditions.

### A. Inducible Promoters

An inducible promoter is operably linked to a gene for expression in potato. Optionally, the inducible promoter is operably linked to a nucleotide sequence encoding a signal sequence which is operably linked to a gene for expression in potato. With an inducible promoter the rate of transcription increases in response to an inducing agent.

Any inducible promoter can be used in the instant disclosure. See Ward et al., Plant Mol. Biol. 22:361-366 (1993). Exemplary inducible promoters include, but are not limited to, that from the ACEI system which responds to copper (Mett et al., PNAS 90:4567-4571 (1993)); In2 gene from maize which responds to benzenesulfonamide herbicide safeners (Hershey et al., Mol. Gen Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone. Schena et al., Proc. Natl. Acad. Sci. USA 88:0421 (1991).

### B. Constitutive Promoters

A constitutive promoter is operably linked to a gene for expression in potato or the constitutive promoter is operably linked to a nucleotide sequence encoding a signal sequence which is operably linked to a gene for expression in potato.

Many different constitutive promoters can be utilized in the instant disclosure. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2: 163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)).

The ALS promoter, Xbal/Ncol fragment 5′ to the Brassica napus ALS3 structural gene (or a nucleotide sequence similarity to said Xbal/Ncol fragment), represents a particularly useful constitutive promoter. See PCT application WO 96/30530.

### C. Tissue-Specific or Tissue-Preferred Promoters

A tissue-specific promoter is operably linked to a gene for expression in potato. Optionally, the tissue-specific promoter is operably linked to a nucleotide sequence encoding a signal sequence which is operably linked to a gene for expression in potato. Plants transformed with a gene of interest operably linked to a tissue-specific promoter produce the protein product of the transgene exclusively, or preferentially, in a specific tissue.

Any tissue-specific or tissue-preferred promoter can be utilized in the instant disclosure. Exemplary tissue-specific or tissue-preferred promoters include, but are not limited to, a root-preferred promoter—such as that from the phaseolin gene (Murai et al., Science 23:476-482 (1983) and Sengupta-Gopalan et al., Proc. Natl. Acad. Sci. USA 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

### Signal Sequences for Targeting Proteins to Subcellular Compartments

Transport of protein produced by transgenes to a subcellular compartment such as the chloroplast, vacuole, peroxisome, glyoxysome, cell wall or mitochondrion or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized.

The presence of a signal sequence directs a polypeptide to either an intracellular organelle or subcellular compartment or for secretion to the apoplast. Many signal sequences are known in the art. See, for example, Becker et al., Plant Mol. Biol. 20:49 (1992); Close, P. S., Master's Thesis, Iowa State University (1993); Knox, C., et al., Plant Mol. Biol. 9:3-17 (1987); Lerner et al., Plant Physiol. 91:124-129 (1989); Frontes et al., Plant Cell 3:483-496 (1991); Matsuoka et al., Proc. Natl. Acad. Sci. 88:834 (1991); Gould et al., J. Cell. Biol. 108:1657 (1989); Creissen et al., Plant J. 2:129 (1991); Kalderon, et al., Cell 39:499-509 (1984); Steifel, et al., Plant Cell 2:785-793 (1990).

### Taxonomy of the Genus Solanum

The Solanaceae family contains several well-known cultivated crops such as tomato (Solanum lycopersicum also referred to as Lycopersicon esculentum), eggplant (Solanum melogena), tobacco (Nicotiana tabacum), pepper (Capsicum annuum) and potato (Solanum tuberosum). Within the genus Solanum, over a thousand species have been recognized. Potatoes will not hybridize with non-tuber bearing Solanum (tomato, eggplant, etc.) species including weeds commonly found in and around commercial potato fields (Love 1994).

The genus Solanum is divided into several subsections, of which the subsection potatoe contains all tuber-bearing potatoes. The subsection potatoe is divided into series, of which tuberosa is relevant to this document. Within the series tuberosa approximately 54 species of wild and cultivated potatoes are found. One of these is S. tuberosum.

S. tuberosum is divided into two subspecies: tuberosum and andigena. The subspecies tuberosum is the cultivated potato widely in use as a crop plant in, for example, North America and Europe. The subspecies andigena is also a cultivated species, but cultivation is restricted to Central and South America (Hanneman 1994).

### Wild Potatoes in the U.S.

The only two wild potato species that grow within the borders of the USA, and for which specimens exist in gene banks, include the tetraploid species S. fendleri (recently reclassified as S. stoloniferum; however, some sources, including the Inter-genebank Potato Database, still use the S. fendleri designation) and the diploid species S. jamesii (Bamberg et al. 2003; IPD 2011; Bamberg and del Rio 2011a; Bamberg and del Rio 2011b; Spooner et al. 2004). Love (1994) reported that a third species, S. pinnatisectum, is also a native species in the USA. However, Spooner et al. (2004) determined that what was previously thought to be S. pinnatisectum was in fact S. jamesii. Through more than 10 years of field work and assessments of existing records, Bamberg et al. (2003) and Spooner et al. (2004) established the presence of only these two species, S. fendleri and S. jamesii, in the U.S. These researchers also attempted to verify previously recorded locations, and through this process, updated the maps of current known locations of these species, providing latitude and longitude locations for each documented population (Bamberg et al. 2003) and distribution maps (Spooner et al. 2004). These species mostly reside in dry forests, scrub desert, and sandy areas at altitudes of 5,000 to 10,000 feet, well isolated from most commercial production areas (Bamberg and del Rio 2011a).

While there is some overlap between the acreage used for commercial production and occurrence of wild species on a county level, the majority of the potato production in the United States is not in wild potato zones. However, there is a possibility that a few wild potato plants may be growing near potato fields (Love 1994). Spooner et al. (2004) describe S. jamesii habitat in the U.S. as among boulders on hillsides, sandy alluvial stream bottoms, in gravel along trails or roadways, rich organic soil of alluvial valleys, sandy fallow fields, grasslands, juniper-pinyon scrub deserts, oak thicket, coniferous and deciduous forests at elevations between 4,500 to 9,400 feet. They describe S. fendleri habitat similarly, and at elevations between 4700 to 11,200 feet.

### Genetics of Potato

The basic chromosome number in the genus Solanum is twelve. S. tuberosum subsp. tuberosum can be diploids (2n=2x=24) or tetraploids (2n=4x=48). The diploids have a limited range in parts of South America, while the tetraploids are the most commonly cultivated all over the world. How tetraploidy originated in potato is unclear. The cultivated S. tuberosum subsp. tuberosum can be either an autotetraploid (doubling of the chromosomes of a diploid species) or an allotetraploid (doubling of the chromosomes of a diploid hybrid between two related species).

While nearly all diploid species are self-incompatible, the cultivated tetraploid S. tuberosum subsp. tuberosum is capable of self-pollination (selfing). Plaisted (1980) has shown that under field conditions selfing is most likely for tetraploid S. tuberosum, with 80-100 percent of the seeds formed due to selfing. Conner and Dale (1996) collected outcrossing data from several field experiments with genetically modified potatoes, performed in New Zealand, the United Kingdom and Sweden. In each study, the outcrossing rate was zero when receiving plants were separated by more than 20 meters from the genetically modified ones. Although many Solanum species are fertile, it appears that a large number of the tetraploid cultivated S. tuberosum subsp. tuberosum cultivars have reduced fertility.

### Potato Varieties

Potato varieties take many years to develop. The decision to establish a new variety is based on many factors such as need in the market place, potential consumer acceptance, and pest tolerance or resistance. Potato varieties do not have a high frequency of introduction and discontinuation compared to some other crops such as field corn or soybeans. Since potatoes are clonally propagated, there is a reduced risk of varietal dilution due to cross pollination.

The potato events used in the present disclosure originate from four potato varieties.

Russet Burbank is the parent variety for event E12 and W8. Luther Burbank developed this variety in the early 1870s. Plants are vigorous and continue vine growth throughout the season. Stems are thick, prominently angled and finely mottled. Leaflets are long to medium in width and light to medium green in color. The blossoms are few, white and not fertile. The cultivar is tolerant to common scab but is susceptible to Fusarium and Verticillium wilts, leafroll and net necrosis and virus Y. Plants require conditions of high and uniform soil moisture and controlled nitrogen fertility to produce tubers free from knobs, pointed ends and dumbbells. Jelly-end and sugar-end develop in tubers when plants are subjected to stress. The tubers produced are large brown-skinned and white-fleshed, display good long-term storage characteristics, and represent the standard for excellent baking and processing quality. The variety is sterile and widely grown in the Northwest and Midwest, especially for the production of french fries.

Ranger Russet is the parent variety for event F10 and X17. This full season variety was released in 1991. Ranger Russet is more resistant than Russet Burbank to Verticillium wilt, viruses X and Y, leafroll and net necrosis, and Fusarium dry rot. It is highly resistant to hollow heart. Plants are large and upright to spreading. Stems are thick, green that can be light brownish to light purple in full sun. Leaves are large, broad and medium green. Flowers are abundant and produce viable pollen. Buds are green with reddish-purple base and pedicel and moderate amount of short pubescence. Corolla is medium large, red-purple color and anthers are bright yellow. It produces high yields of good quality, high specific gravity tubers that are long and slightly flattened, and well suited for baking and processing into french fries. Tubers are susceptible to common scab and black spot bruise. Ranger Russet matures earlier than Russet Burbank and would be considered a medium-length storage variety. The variety is fertile and mainly grown in the Northwest, especially for the production of french fries.

Atlantic is the parent variety for event J3 and J55 and Y9. Plants are moderately large, with thick, upright stems, and slightly swollen, sparsely pubescent nodes. Leaves are bright, medium green, smooth, and moderately pubescent with prominent wings, large asymmetrical primary leaflets and numerous secondary and tertiary leaflets. Flowers are profuse with green, awl-shaped, pubescent calyx lobes, pale lavender corolla, orange anthers and abundant, viable pollen. The cultivar is tolerant to scab and Verticillium wilt, resistant to pinkeye, highly resistant to Race A of golden nematode, virus X, tuber net necrosis, and shows some resistance to black spot bruise. Tubers are susceptible to internal heat necrosis, particularly in sandy soils in warm, dry seasons. Hollow heart in the larger diameter tubers (diameter >4 inches) can be serious in some growing areas. Tubers are oval to round with light to heavy scaly netted skin, moderately shallow eyes, and white flesh. Tuber dormancy is medium-long. With high yield potential, high specific gravity and uniform tuber size and shape, Atlantic is the standard variety for chipping from the field or from very short-term storage (Webb et al. 1978). The variety is fertile and mainly grown in the Northeast and Southeast, especially for the production of chips.

Snowden is the parent variety for event V11. Snowden (W 855) was selected in the late 1970s in Wisconsin from a cross between Wischip and B5141-6, and named in 1990. Selection and early testing was done by Dr. Stan Peloquin and Mr. Donald Kichefski at the UW-Lelah Starks Potato Breeding Farm, Rhinelander, Wis. Vines are large erect and medium. Leaves are light green and closed. Flowers are white with yellow anthers and tend to abort. Male sterility is common and fruit rarely develop. The eyes are medium, deeper at the apical end and uniformly distributed. The tuber has white flesh, while the skin is light tan slightly netted. The tuber is uniform, round and slightly flat, with consistently 2.5 to 3.5 inch diameter. It is susceptible to early and late blights and common scab and is attractive to the Colorado potato beetle. The variety is tolerant to hollow heart and brown center. It yields slightly less than or about the same as Atlantic. It is now a standard in the North Central Regional Trials. It is very much like Atlantic except that it chips out of 45° F. storage without reconditioning. It is used for the production of chips.

### Methods for Potato Transformation

Numerous methods for plant transformation have been developed, including biological and physical plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc. Boca Raton, 1993) pages 67-88. In addition, expression vectors and in-vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pages 89-119.

### A. Agrobacterium-Mediated Transformation

One method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci. 10:1 (1991). Descriptions of Agrobacterium vector systems and methods for Agrobacterium-mediated gene transfer are provided by Gruber et al., supra, Miki et al., supra and Moloney et al., Plant Cell Reports 8:238 (1989). See also, U.S. Pat. No. 5,563,055 (Townsend and Thomas), issued Oct. 8, 1996. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. No. 5,693,512, U.S. Pat. No. 6,051,757 and EP904362A1, which are all hereby incorporated by reference in their entirety.

Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method.

Agrobacterium-mediated transformation is achieved through the use of a genetically engineered soil bacterium belonging to the genus Agrobacterium. Several Agrobacterium species mediate the transfer of a specific DNA known as “T-DNA” that can be genetically engineered to carry any desired piece of DNA into many plant species. The major events marking the process of T-DNA mediated pathogenesis are: induction of virulence genes, processing and transfer of T-DNA. This process is the subject of many reviews (Ream, 1989; Howard and Citovsky, 1990; Kado, 1991; Hooykaas and Schilperoort, 1992; Winnans, 1992; Zambryski, 1992; Gelvin, 1993; Binns and Howitz, 1994; Hooykaas and Beijersbergen 1994; Lessl and Lanka, 1994; Zupan and Zambryski, 1995).

Agrobacterium-mediated genetic transformation of plants involves several steps. The first step, in which the Agrobacterium and plant cells are first brought into contact with each other, is generally called “inoculation”. Following the inoculation step, the Agrobacterium and plant cells/tissues are usually grown together for a period of several hours to several days or more under conditions suitable for growth and T-DNA transfer. This step is termed “co-culture”. Following co-culture and T-DNA delivery, the plant cells are often treated with bacteriocidal and-or bacteriostatic agents to kill the Agrobacterium. If this is done in the absence of any selective agents to promote preferential growth of transgenic versus non-transgenic plant cells, then this is typically referred to as the “delay” step. If done in the presence of selective pressure favoring transgenic plant cells, then it is referred to as a “selection” step. When a “delay” is used, it is followed by one or more “selection” steps. Both the “delay” and “selection”. steps typically include bactericidal and-or bacteriostatic agents to kill any remaining Agrobacterium cells because the growth of Agrobacterium cells is undesirable after the infection (inoculation and co-culture) process.

Although transgenic plants produced through Agrobacterium-mediated transformation generally contain a simple integration pattern as compared to microparticle-mediated genetic transformation, a wide variation in copy number and insertion patterns exists (Jones et al, 1987; Jorgensen et al., 1987). Moreover, even within a single plant genotype, different patterns of T-DNA integration are possible based on the type of explant and transformation system used (Grevelding et al., 1993). Factors that regulate T-DNA copy number are poorly understood.

The particular Innate™ methodology of transformation will be set forth in the accompanying examples. However, the aforementioned “generic” method of Agrobacterium-mediated plant transformation of plants also produces non-naturally occurring nucleotide junction sequences that can be detected by the taught methods.

### B. Direct Gene Transfer

Direct plant transformation methods using DNA have also been reported. The first of these to be reported historically is electroporation, which utilizes an electrical current applied to a solution containing plant cells (M. E. Fromm et al., Nature, 319, 791 (1986); H. Jones et al., Plant Mol. Biol., 13, 501 (1989) and H. Yang et al., Plant Cell Reports, 7, 421 (1988).

Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope, but without killing at least some of them (U.S. Pat. No. 5,204,253, U.S. Pat. No. 5,015,580).

A third direct method uses fibrous forms of metal or ceramic consisting of sharp, porous or hollow needle-like projections that literally impale the cells, and also the nuclear envelope of cells. Both silicon carbide and aluminum borate whiskers have been used for plant transformation (Mizuno et al., 2004; Petolino et al., 2000; U.S. Pat. No. 5,302,523 US Application 20040197909) and also for bacterial and animal transformation (Kaepler et al., 1992; Raloff, 1990; Wang, 1995). There are other methods reported, and undoubtedly, additional methods will be developed. The methods taught herein are capable of detecting the non-naturally occurring nucleotide junctions that result from any plant transformation method.

### Potato Breeding

The foregoing methods for transformation would typically be used for producing a transgenic variety. The transgenic variety could then be crossed with another (non-transformed or transformed) variety in order to produce a new transgenic variety. Alternatively, a genetic trait that has been engineered into a particular potato line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties that do not contain that gene. As used herein, “crossing” can refer to a simple X by Y cross or the process of backcrossing depending on the context.

Persons of ordinary skill in the art will recognize that when the term potato plant is used in the context of the present disclosure, this also includes derivative varieties that retain the essential distinguishing characteristics of the event in question, such as a gene converted plant of that variety or a transgenic derivative having one or more value-added genes incorporated therein (such as herbicide or pest resistance). Backcrossing methods can be used with the present disclosure to improve or introduce a characteristic into the variety. The term “backcrossing” as used herein refers to the repeated crossing 1, 2, 3, 4, 5, 6, 7, 8, 9 or more times of a hybrid progeny back to the recurrent parents. The parental potato plant which contributes the gene(s) for the one or more desired characteristics is termed the nonrecurrent or donor parent. This terminology refers to the fact that the nonrecurrent parent is used one time in the backcross protocol and therefore does not recur. The parental potato plant to which the gene or genes from the nonrecurrent parent are transferred is known as the recurrent parent as it is used for several rounds in the backcrossing protocol. In a typical backcross protocol, the original variety of interest (recurrent parent) is crossed to a second variety (nonrecurrent parent) that carries the gene(s) of interest to be transferred. The resulting progeny from this cross are then crossed again to the recurrent parent and the process is repeated until a potato plant is obtained wherein essentially all of the desired morphological and physiological characteristics of the recurrent parent are recovered in the converted plant, in addition to the one or more genes transferred from the nonrecurrent parent.

The selection of a suitable recurrent parent is an important step for a successful backcrossing procedure. The goal of a backcross protocol is to alter or substitute one or more traits or characteristics in the original variety. To accomplish this, one or more genes of the recurrent variety are modified, substituted or supplemented with the desired gene(s) from the nonrecurrent parent, while retaining essentially all of the rest of the desired genes, and therefore the desired physiological and morphological constitution of the original variety. The choice of the particular nonrecurrent parent will depend on the purpose of the backcross. One of the major purposes is to add some commercially desirable, agronomically important trait to the plant. The exact backcrossing protocol will depend on the characteristic or trait being altered or added to determine an appropriate testing protocol. Although backcrossing methods are simplified when the characteristic being transferred is a dominant allele, a recessive allele may also be transferred. In this instance, it may be necessary to introduce a test of the progeny to determine if the desired characteristic has been successfully transferred.

Likewise, transgenes can be introduced into the plant using any of a variety of established recombinant methods well-known to persons skilled in the art, such as: Gressel, 1985, Biotechnologically Conferring Herbicide Resistance in Crops: The Present Realities, In Molecular Form and Function of the Plant Genome, L. van Vloten-Doting, (ed.), Plenum Press, New York; Huttner, S. L., et al., 1992, Revising Oversight of Genetically Modified Plants, Bio/Technology; Klee, H., et al., 1989, Plant Gene Vectors and Genetic Transformation: Plant Transformation Systems Based on the use of Agrobacterium tumefaciens, Cell Culture and Somatic Cell Genetics of Plants; Koncz, C., et al., 1986, The Promoter of T.sub.L-DNA Gene 5 Controls the Tissue-Specific Expression of Chimeric Genes Carried by a Novel Type of Agrobacterium Binary Vector; Molecular and General Genetics; Lawson, C., et al., 1990, Engineering Resistance to Mixed Virus Infection in a Commercial Potato Cultivar: Resistance to Potato Virus X and Potato Virus Y in Transgenic Russet Burbank, Bio/Technology; Mitsky, T. A., et al., 1996, Plants Resistant to Infection by PLRV. U.S. Pat. No. 5,510,253; Newell, C. A., et al., 1991, Agrobacterium-Mediated Transformation of Solanum tuberosum L. Cv. Russet Burbank, Plant Cell Reports; Perlak, F. J., et al., 1993, Genetically Improved Potatoes: Protection from Damage by Colorado Potato Beetles, Plant Molecular Biology; all of which are incorporated herein by reference for this purpose.

Many traits have been identified that are not regularly selected for in the development of a new variety but that can be improved by backcrossing and genetic engineering techniques. These traits may or may not be transgenic; examples of these traits include but are not limited to: herbicide resistance; resistance to bacterial, fungal or viral disease; insect resistance; uniformity or increase in concentration of starch and other carbohydrates; enhanced nutritional quality; decrease in tendency of tuber to bruise; and decrease in the rate of starch conversion to sugars. These genes are generally inherited through the nucleus.

### Deposit Information

The following deposit information is included merely to provide representative plant material used in the examples and claimed methods.

A tuber deposit of the J.R. Simplot Company proprietary POTATO CULTIVAR J3 disclosed above has been made with the American Type Culture Collection (ATCC), 10801 University Boulevard, Manassas, Va. 20110. The date of deposit was May 23, 2013. The ATCC Accession Number is PTA-120371. See, U.S. Pat. No. 8,754,303, “Potato Cultivar J3” incorporated herein by reference.

A tuber deposit of the J.R. Simplot Company proprietary POTATO CULTIVAR F10 disclosed above has been made with the American Type Culture Collection (ATCC), 10801 University Boulevard, Manassas, Va. 20110. The date of deposit was May 23, 2013. The ATCC Accession Number is PTA-120373. See, U.S. Pat. No. 8,710,311 “Potato Cultivar F10” incorporated herein by reference.

A tuber deposit of the J.R. Simplot Company proprietary POTATO CULTIVAR W8 disclosed above has been made with the American Type Culture Collection (ATCC), 10801 University Boulevard, Manassas, Va. 20110. The date of deposit was Mar. 11, 2014. The ATCC Accession Number is PTA-121079. See, U.S. Pat. No. 8,889,964 “Potato Cultivar W8” incorporated herein by reference.

A tuber deposit of the J. R. Simplot Company proprietary POTATO CULTIVAR J55 disclosed above has been made with the American Type Culture Collection (ATCC), 10801 University Boulevard, Manassas, Va. 20110. The date of deposit was Sep. 25, 2013. The ATCC Accession Number is PTA-120601. See, U.S. Pat. No. 8,889,963 “Potato Cultivar J55” incorporated herein by reference.

A tuber deposit of the J.R. Simplot Company proprietary POTATO CULTIVAR E12 disclosed above has been made with the American Type Culture Collection (ATCC), 10801 University Boulevard, Manassas, Va. 20110. The date of deposit was May 23, 2013. The ATCC Accession Number is PTA-120372. See, U.S. patent application Ser. No. 14/072,487 “Potato Cultivar E12” incorporated herein by reference.

A tuber deposit of the J. R. Simplot Company proprietary POTATO CULTIVAR X17 disclosed above has been made with the American Type Culture Collection (ATCC), 10801 University Boulevard, Manassas, Va. 20110. The date of deposit was Jun. 17, 2015. The ATCC Accession Number is PTA-122248.

A tuber deposit of the J. R. Simplot Company proprietary POTATO CULTIVAR Y9 disclosed above has been made with the American Type Culture Collection (ATCC), 10801 University Boulevard, Manassas, Va. 20110. The date of deposit was Jun. 17, 2015. The ATCC Accession Number is PTA-122247.

A tuber deposit of the J. R. Simplot Company proprietary POTATO CULTIVAR V11 disclosed above has been made with the American Type Culture Collection (ATCC), 10801 University Boulevard, Manassas, Va. 20110. The date of deposit was ______. The ATCC Accession Number is ______.

### EXAMPLES

### Example 1

### The pSIM1278 Transformation Vector Backbone

Plasmid pSIM1278 is a 19.7 kb binary transformation vector used to transform potatoes. This example shows the source of the genetic elements, the cloning steps for the backbone, and T-DNA sequences, and the order of the elements in the plasmid.

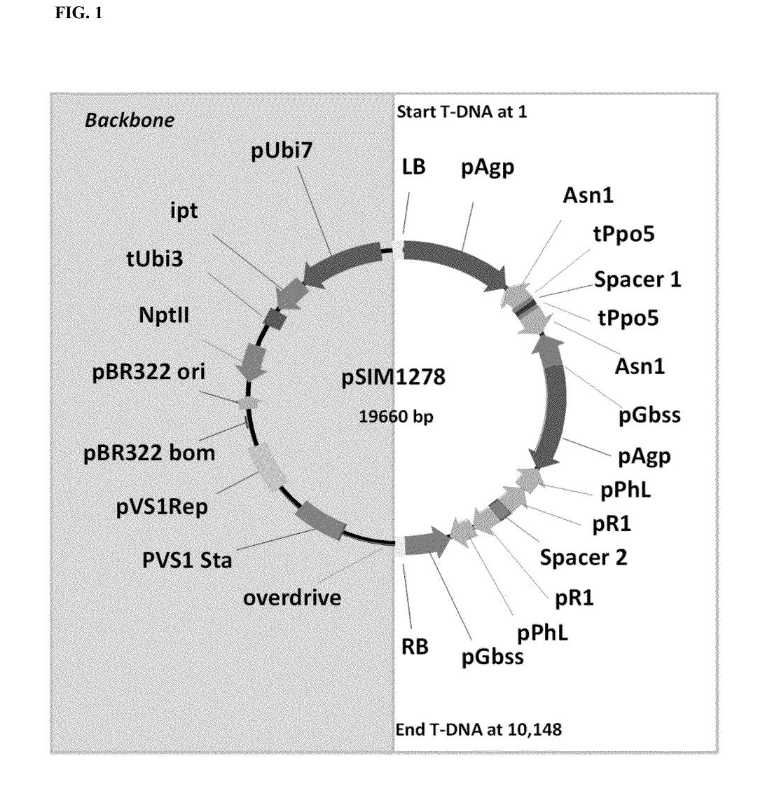

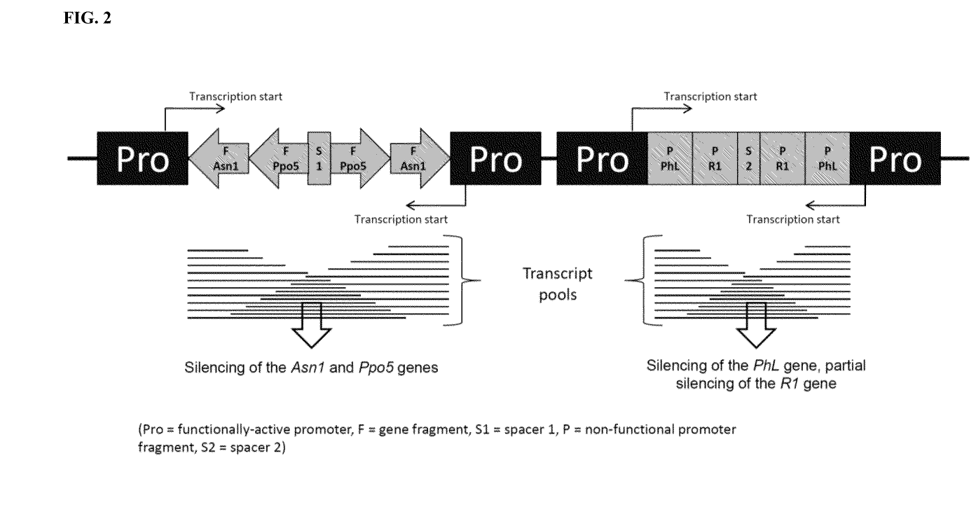

The plasmid backbone (FIG. 1 and Table 1) contains two well-characterized bacterial origins of replication. pVS1 (pVS1 Sta and Rep) enables maintenance of the plasmid in Agrobacterium, and pBR322 (pBR322 bom and ori) enables maintenance of the plasmid in Escherichia coli. The Agrobacterium DNA overdrive sequence enhances cleavage at the RB, and the E. coli. nptII gene is a bacterial kanamycin selectable marker. The backbone contains an expression cassette comprising the Agrobacterium isopentenyl transferase (ipt) gene flanked by the Ranger Russet potato polyubiquitin (Ubi7) promoter and the Ranger Russet potato polyubiquitin (Ubi3) terminator. The ipt cassette is a screenable phenotype used to select against plasmid backbone DNA integration in the host plant. When present in transformed plant tissue, overexpression of ipt results in the overproduction of the plant hormone cytokinin resulting in plants with stunted phenotypes, abnormal leaves and the inability to root.

The backbone portion is not transferred into the plant cells. The various elements of the backbone are described in Table 1.

TABLE 1
Genetic Elements of the pSIM1278 Backbone

| | | Accession | | Size | |
|---|---|---|---|---|---|
| Genetic Element | Origin | Number¹ | Position | (bp) | Function |
| 1. Intervening sequence | Synthetic DNA | | 10,149-10,154 | 6 | Sequence used for cloning |
| 2. Overdrive | Agrobacterium | NC_002377 | 10,155-10,187 | 33 | Enhances cleavage of A. tumefaciens |
| | tumefaciens | | | | Right Border site¹ |
| | Ti-plasmid | | | | |
| 3. Intervening sequence | Pseudomonas | AJ537514 | 10,188-11,266 | 1,079 | pVS1 backbone¹ |
| | fluorescens | | | | |
| | pVS1 | | | | |
| 4. pVS1 partitioning | P. fluorescens | AJ537514 | 11,267-12,267 | 1,001 | pVS1 stability¹ |
| protein StaA (PVS1 Sta) | pVS1 | | | | |
| 5. Intervening sequence | P. fluorescens | AJ537514 | 12,268-12,860 | 593 | pVS1 backbone¹ |
| | pVS1 | | | | |
| 6. pVS1 replicon | P. fluorescens | AJ537514 | 12,861-13,861 | 1,001 | pVS1 replication region in |
| (pVS1Rep) | pVS1 | | | | Agrobacterium¹ |
| 7. Intervening sequence | P. fluorescens | AJ537514 | 13,862-14,099 | 238 | pVS1 backbone¹ |
| | pVS1 | | | | |
| 8. Intervening sequence | pBR322 | J01749 | 14,100-14,180 | 81 | pBR322 backbone¹ |
| 9. pBR322 bom | pBR322 | J01749 | 14,181-14,531 | 351 | pBR322 region for replication in E. coli¹ |
| 10. Intervening sequence | pBR322 | J01749 | 14,532-14,670 | 139 | pBR322 backbone¹ |
| 11. Origin of replication | pBR322 | J01749 | 14,671-14,951 | 281 | Bacterial origin of replication¹ |
| for pBR322 (pBR322 ori) | | | | | |
| 12. Intervening sequence | pBR322 | J01749 | 14,952-15,241 | 290 | pBR322 backbone¹ |
| 13. Neomycin | Tn5 transposon | FJ362602 | 15,242-16,036 | 795 | Aminoglycoside |
| phosphotransferase II | | | | | phosphotransferase¹ (Simpson et al. |
| (nptII) gene | | | | | 1985) |
| 14. Intervening sequence | Vector DNA | FJ362602 | 16,037-16,231 | 195 | pCAMBIA vector backbone¹ |
| 15. Terminator of the | S. tuberosum | GP755544 | 16,232-16,586 | 355 | Terminator for ipt gene transcription |
| ubiquitin-3 gene (tUbi3) | | | | | (Garbarino and Belknap, 1994) |
| 16. Intervening sequence | A. tumefaciens | NC_002377 | 16,587-16,937 | 351 | Sequence used for DNA cloning |
| | Ti-plasmid | | | | |
| 17. Isopentenyl | A. tumefaciens | NC_002377 | 16,938-17,660 | 723 | Condensation of AMP and |
| transferase (ipt) gene | Ti-plasmid | | | | isopentenyl-pyrophosphate to form |
| | | | | | isopentenyl-AMP, a cytokinin in the |
| | | | | | plant. Results in abnormal growth |
| | | | | | phenotypes in plant (Smigocki and |
| | | | | | Owens, 1988) |
| 18. Intervening sequence | Synthetic DNA | | 17,661-17,672 | 12 | Sequence used for DNA cloning |
| 19. Polyubiquitin | S. tuberosum | U26831 | 17,673-19,410 | 1,738 | Promoter to drive expression of the |
| promoter (Ubi7) | var. Ranger | | | | ipt backbone marker gene |
| | Russet | | | | (Garbarino et al., 1995) |
| 20. Intervening sequence | Vector DNA | U10460 | 19,411-19,660 | 250 | pZP200 vector backbone¹ |

¹http://www.cambia.org/daisy/cambia/585.html-(General structure map of pCAMBIA vectors)

### Example 2

### The pSIM1278 Transformation Vector T-DNA

The pSIM1278 DNA insert region, including the flanking border sequences, used in the pSIM1278 is 10,148 bp long, from 1 bp to 10,148 bp. The pSIM1278 DNA insert consists of native DNA only and is stably integrated into the potato genome. The pSIM1278 DNA insert or a functional part thereof, is the only genetic material of vector pSIM1278 that is integrated in the potato plant varieties of the invention.

The pSIM1278 DNA insert is described in: FIG. 1 (along with vector backbone region), FIG. 2, FIG. 5, and Table 2 below. The LB and RB sequences (25 bp each) were synthetically designed to be similar to and function like T-DNA borders from Agrobacterium tumefaciens. The GenBank Accession AY566555 was revised to clarify the sources of DNA for the Border regions. ASN1 described as genetic elements 5 and 10 is referred to as StAst1 in Chawla et al., 2012.

Plasmid pSIM1278 T-DNA contains two expression cassettes:

The first cassette (elements 4 to 12, Table 2) results in down-regulation of Asn1 and Ppo5 in the transformed potato variety. It is comprised of two identical 405 bp fragments of Asn1 and two identical 144 bp fragments of Ppo5. The fragments of Asn1 and Ppo5 are arranged as inverted repeats separated by a non-coding 157 bp Ranger Russet potato nucleotide spacer element. The Asn1 and Ppo5 fragments are arranged between the two convergent potato promoters; the Agp promoter of the ADP glucose pyrophosphorylase gene (Agp) and the Gbss promoter of the granule-bound starch synthase gene (Gbss) that are primarily active in tubers. These promoters drive expression of the inverted repeats to generate double-stranded RNA and down-regulate Asn1 and Ppo5.

The second cassette (elements 14 to 21, Table 2) results in down-regulation of PhL and R1 in the transformed potato variety. It is comprised of two identical 509 bp fragments of the PhL promoter region (pPhL) and two identical 532 bp fragments of R1 promoter region (pR1). The pPhL and pR1 fragments are arranged as inverted repeats separated by a non-coding 258 bp fragment of the Ranger Russet potato polyubiquitin gene. Like the first cassette, the pPhL and pR1 fragments are arranged between and transcribed by the potato Agp and Gbss promoters.

TABLE 2
Genetic Elements of pSIM1278 T-DNA, from Left Border Site to Right Border

| | | Accession | Position | Size | |
|---|---|---|---|---|---|
| Genetic Element | Origin | Number | (pSIM1278) | (bp) | Intended Function |
| 1. Left Border (LB) site¹ | Synthetic | AY566555⁵ | 1-25 | 25 | Site for secondary cleavage to release |
| | | (bases 1-25) | | | single-stranded DNA insert from |
| | | | | | pSIM1278 (van Haaren et al. 1989) |
| 2. Left Border region | S. tuberosum | AY566555⁵ | 26-187 | 162 | Supports secondary cleavage at LB |
| sequence including LB | var. | (bases 26-187) | | | |
| | Ranger Russet. | | | | |
| 3. Intervening Sequence | S. tuberosum | AF393847 | 188-193 | 6 | Sequence used for DNA cloning |
| 4. Promoter for the ADP | S. tuberosum | HM363752 | 194-2,453 | 2260 | One of the two convergent promoters that |
| glucose pyrophosphorylase | var. | | | | drives expression of an inverted repeat |
| gene (pAgp), 1st copy | Ranger Russet | | | | containing fragments of Asn1 and Ppo5, |
| | | | | | especially in tubers |
| 5. Fragment of the | S. tuberosum | HM363759 | 2,454-2,858 | 405 | Generates with (11) double stranded RNA |
| asparagine synthetase-1 | var. | | | | that triggers the degradation of Asn1 |
| (Asn1) gene (1st copy | Ranger Russet | | | | transcripts to impair asparagine formation |
| antisense orientation) | | | | | (Chawla et al., 2012²) |
| 6. 3′-untranslated sequence | S. verrucosum | HM363754 | 2,859-3,002 | 144 | Generates with (9) double stranded RNA |
| of the polyphenol oxidase- | | | | | that triggers the degradation of Ppo5 |
| 5 gene (Ppo5) (1st copy, in | | | | | transcripts to block black spot |
| antisense orientation) | | | | | development |
| 7. Intervening Sequence | S. tuberosum | DQ478950 | 3,003-3,008 | 6 | Sequence used for DNA cloning |
| 8. Spacer-1 | S. tuberosum | HM363753 | 3,009-3,165 | 157 | Sequence between the 1st inverted repeats |
| | var. | | | | |
| | Ranger Russet | | | | |
| 9. 3′-untranslated sequence | S. verrucosum | HM363754 | 3,166-3,309 | 144 | Generates with (6) double stranded RNA |
| of the polyphenol oxidase- | | | | | that triggers the degradation of Ppo5 |
| 5 gene (Ppo5) (2nd copy, | | | | | transcripts to block black spot |
| in sense orientation) | | | | | development |
| 10. Fragment of the | S. tuberosum | HM363759 | 3,310-3,715 | 406 | Generates with (5) double stranded RNA |
| asparagine synthetase-1 | var. | | | | that triggers the degradation of Asn1 |
| (Asn1) gene (2nd copy, in | Ranger Russet | | | | transcripts to impair asparagine formation |
| sense orientation) | | | | | (Chawla et al., 2012²) |
| 11. Intervening Sequence | S. tuberosum | X73477 | 3,716-3,721 | 6 | Sequence used for DNA cloning |
| 12. Promoter for the | S. tuberosum | HM363755 | 3,722-4,407 | 686 | One of the two convergent promoters that |
| granule-bound starch | var. | | | | drives expression of an inverted repeat |
| synthase (pGbss) gene (1st | Ranger Russet | | | | containing fragments of Asn1 and Ppo5, |
| copy, convergent | | | | | especially in tubers |
| orientation relative to the | | | | | |
| 1st copy of pAgp) | | | | | |
| 13. Intervening Sequence | S. tuberosum | X95996/ | 4,408-4,423 | 16 | Sequence used for DNA cloning |
| | | AF393847 | | | |
| 14. pAgp, 2nd copy | S. tuberosum | HM363752 | 4,424-6,683 | 2260 | One of the two convergent promoters that |
| | var. | | | | drives expression of an inverted repeat |
| | Ranger Russet | | | | containing fragments of the promoters of |
| | | | | | PhL and R1, especially in tubers |
| 15. Fragment of promoter | S. tuberosum | HM363758 | 6,684-7,192 | 509 | Generates with (21) double stranded RNA |
| for the potato | var. | | | | that triggers the degradation of PhL |
| phosphorylase-L (pPhL) | Ranger Russet | | | | transcripts to limit the formation of |
| gene (1st copy, in | | | | | reducing sugars through starch |
| antisense orientation) | | | | | degradation |
| 16. Fragment of promoter | S. tuberosum | HM363757 | 7,193-7,724 | 532 | Generates with (20) double stranded RNA |
| for the potato R1 gene | var. | | | | that triggers the degradation of R1 |
| (pR1) (1st copy, in | Ranger Russet | | | | transcripts to limit the formation of |
| antisense orientation) | | | | | reducing sugars through starch |
| | | | | | degradation |
| 17. Intervening Sequence | S. tuberosum | DQ478950 | 7,725-7,730 | 6 | Sequence used for DNA cloning |
| 18. Spacer-2 | S. tuberosum | U26831³ | 7,731-7,988 | 258 | Sequence between the 2nd inverted repeat |
| | var. | | | | |
| | Ranger Russet | | | | |
| 19. Fragment of promoter | S. tuberosum | HM363757 | 7,989-8,520 | 532 | Generates with (20) double stranded RNA |
| for the potato R1 gene | var. | | | | that triggers the degradation of R1 |
| (pR1) (2nd copy, in sense | Ranger Russet | | | | transcripts to limit the formation of |
| orientation) | | | | | reducing sugars through starch |
| | | | | | degradation |
| 20. Fragment of promoter | S. tuberosum | HM363758 | 8,521-9,029 | 509 | Generates with (16) double stranded RNA |
| for the potato | var. | | | | that triggers the degradation of PhL |
| phosphorylase-L (pPhL) | Ranger Russet | | | | transcript to limit the formation of |
| gene (2nd copy, in sense | | | | | reducing sugars through starch |
| orientation) | | | | | degradation |
| 21. pGbss (2nd copy, | S. tuberosum | X83220⁴ | 9,030-9,953 | 924 | One of the two convergent promoters that |
| convergent orientation | var. | | | | drives expression of an inverted repeat |
| relative to the 2nd copy of | Ranger Russet | | | | containing fragments of the promoters of |
| pAgp) | | | | | PhL and R1, especially in tubers |
| 22. Intervening Sequence | S. tuberosum | AF143202 | 9,954-9,962 | 9 | Sequence used for DNA cloning |
| 23. Right Border region | S. tuberosum | AY566555⁵ | 9,963-10,123 | 161 | Supports primary cleavage at RB-Like site |
| sequence including RB | var. | (bases 231-391) | | | |
| | Ranger Russet | | | | |
| 24. Right Border (RB) | Synthetic | AY566555⁵ | 10,124-10,148 | 25 | Site for primary cleavage to release single |
| sequence¹ | | (bases 392-416) | | | stranded DNA insert from pSIM1278 (van |
| | | | | | Haaren et al. 1989) |

¹The LB and RB sequences (25-bp each) were synthetically designed to be similar to and function like T-DNA borders from Agrobacterium tumefaciens.
²ASN1 described as genetic elements 5 and 11 is referred to as StAst1 in Chawla et al. 2012.
³GenBank Accession HM36756H is replaced with a citation to GenBank Accession U26831 to properly include four 3′ end nucleotides present in the pGbss DNA element of the pSIM1278 construct.
⁴GenBank Accession HM363755 is replaced with a citation to GenBank Accession X83220 to properly include the full pGbss (2nd copy) DNA insert sequence present in the pSIM1278 construct.
⁵GenBank Accession AY566555 was revised to clarify the sources of DNA for the Border regions.

Thus, as can be seen from Table 1 and Table 2, the pSIM1278 plasmid is a binary vector designed for potato plant transformation. The vector backbone contains sequences for replication in both E. coli and Agrobacterium along with an ipt marker for screening to eliminate plants with vector backbone DNA. The T-DNA region consists of two expression cassettes flanked by LB and RB sequences. Upon inoculation of host plant tissue with Agrobacterium containing the pSIM1278 vector, the T-DNA region of pSIM1278 is transferred into the host genome.

The DNA insert described in Table 2 that was used to create potato lines of the present disclosure does not activate adjacent genes and does not adversely affect the phenotype of potato plant varieties.

### Example 3

### The pSIM1678 Transformation Vector Backbone

Plasmid pSIM1678 is a 18.6 kb binary transformation vector used to transform potatoes. This example shows the source of the genetic elements, the cloning steps for the backbone, and T-DNA sequences, and the order of the elements in the plasmid.

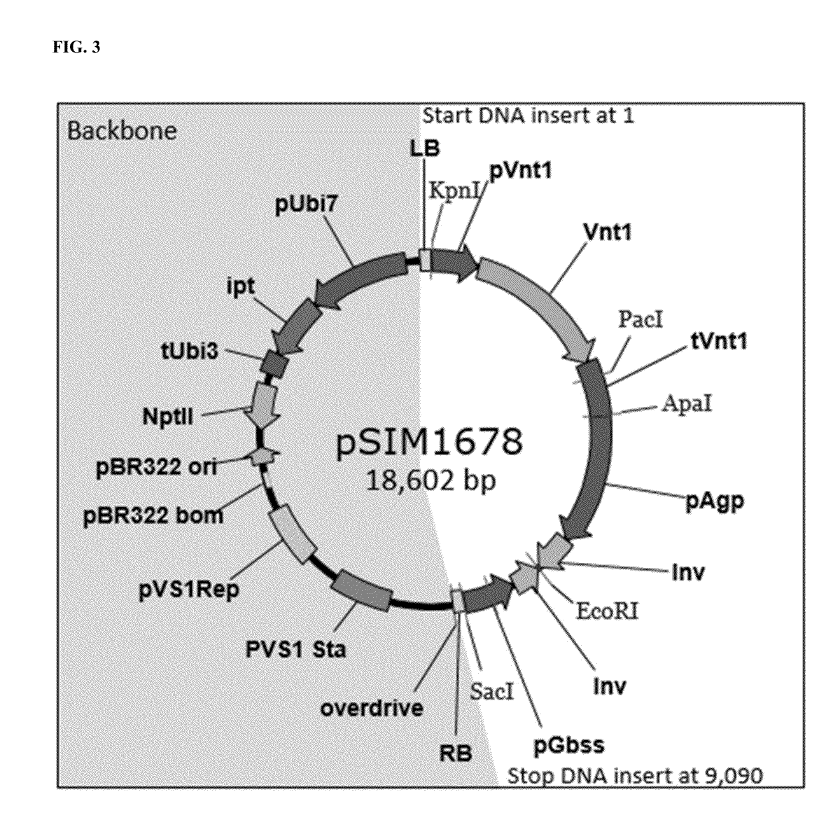

The plasmid backbone (FIG. 3; Table 3) contains two well-characterized bacterial origins of replication. pVS1 (pVS1 Sta and Rep) enables maintenance of the plasmid in Agrobacterium, and pBR322 (pBR322 bom and ori) enables maintenance of the plasmid in Escherichia coli. The Agrobacterium DNA overdrive sequence enhances cleavage at the RB, and the E. coli. nptII gene is a bacterial kanamycin selectable marker. The backbone contains an expression cassette comprising the Agrobacterium isopentenyl transferase (ipt) gene flanked by the Ranger Russet potato polyubiquitin (Ubi7) promoter and the Ranger Russet potato polyubiquitin (Ubi3) terminator (Garbarino and Belknap, 1994). The ipt cassette is a screenable phenotype used to select against plasmid backbone DNA integration in the host plant. When present in transformed plant tissue, overexpression of ipt results in the overproduction of the plant hormone cytokinin resulting in plants with stunted phenotypes, abnormal leaves and the inability to root.

The backbone portion is not transferred into the plant cells. The various elements of the backbone are described in Table 3.

TABLE 3
Genetic Elements of the pSIM1678 Backbone

| | | | | | Function |
|---|---|---|---|---|---|
| | | Accession | | Size | ¹http://www.cambia.org/daisy/cambia/585.html - |
| Genetic Element | Origin | Number¹ | Position | (bp) | (General structure map of pCAMBIA vectors) |
| 1. Intervening sequence | Synthetic DNA | | 9,091-9,096 | 6 | Sequence used for cloning |
| 2. Overdrive | Agrobacterium | NC_002377 | 9,097-9,126 | 30 | Enhances cleavage of A. tumefaciens |
| | tumefaciens | | | | Right Border site¹ |
| | Ti-plasmid | | | | |
| 3. Intervening sequence | Pseudomonas | AJ537514 | 9,127-10,208 | 1,082 | pVS1 backbone¹ |
| | fluorescens pVS1 | | | | |
| 4. pVS1 partitioning protein | P. fluorescens | AJ537514 | 10,209-11,209 | 1,001 | pVS1 stability¹ |
| StaA (PVS1 Sta) | pVS1 | | | | |
| 5. Intervening sequence | P. fluorescens | AJ537514 | 11,210-11,802 | 593 | pVS1 backbone¹ |
| | pVS1 | | | | |
| 6. pVS1 replicon | P. fluorescens | AJ537514 | 11,803-12,803 | 1,001 | pVS1 replication region in |
| (pVS1Rep) | pVS1 | | | | Agrobacterium¹ |
| 7. Intervening sequence | P. fluorescens | AJ537514 | 12,804-13,040 | 237 | pVS1 backbone¹ |
| | pVS1 | | | | |
| 8. Intervening sequence | pBR322 | J01749 | 13,041-13,212 | 172 | pBR322 backbone¹ |
| 9. pBR322 bom | pBR322 | J01749 | 13,213-13,473 | 261 | pBR322 region for replication in E. coli¹ |
| 10. Intervening sequence | pBR322 | J01749 | 13,474-13,612 | 139 | pBR322 backbone¹ |
| 11. Origin of replication for | pBR322 | J01749 | 13,613-13,893 | 281 | Bacterial origin of replication¹ |
| pBR322 (pBR322 ori) | | | | | |
| 12. Intervening sequence | pBR322 | J01749 | 13,894-14,183 | 290 | pBR322 backbone¹ |
| 13. Neomycin | Tn5 transposon | FJ362602 | 14,184-14,978 | 795 | Aminoglycoside phosphotransferase¹ |
| phosphotransferase II (nptII) | | | | | (Simpson et al. 1985) |
| gene | | | | | |
| 14. Intervening sequence | Vector DNA | FJ362602 | 14,979-15,173 | 195 | pCAMBIA vector backbone¹ |
| 15. Terminator of the | S. tuberosum | GP755544 | 15,174-15,528 | 355 | Terminator for ipt gene transcription |
| ubiquitin-3 gene (tUbi3) | | | | | (Garbarino and Belknap, 1994) |
| 16. Intervening sequence | A. tumefaciens | NC_002377 | 15,529-15,879 | 351 | Sequence used for DNA cloning |
| | Ti-plasmid | | | | |
| 17. Isopentenyl transferase | A. tumefaciens | NC_002377 | 15,880-16,602 | 723 | Condensation of AMP and isopentenyl- |
| (ipt) gene | Ti-plasmid | | | | pyrophosphate to form isopentenyl- |
| | | | | | AMP, a cytokinin in the plant. Results |
| | | | | | in abnormal growth phenotypes in plant |
| | | | | | (Smigocki and Owens, 1988) |
| 18. Intervening sequence | Synthetic DNA | | 16,603-16,614 | 12 | Sequence used for DNA cloning |
| 19. Polyubiquitin promoter | S. tuberosum | (A) U26831 | 16,615-18,352 | 1,738 | Promoter to drive expression of the ipt |
| (Ubi7) | var. | | | | backbone marker gene (Garbarino et al., |
| | Ranger Russet | | | | 1995) |
| 20. Intervening sequence | Vector DNA | (B) U10460 | 18,353-18,602 | 250 | pZP200 vector backbone |

### Example 4

### The pSIM1678 Transformation Vector T-DNA

The pSIM1678 DNA insert region, including the flanking border sequences, used in the pSIM1678 is 9,090 bp long (from 1 bp to 9,090 bp). The pSIM1678 DNA insert consists of native DNA only and is stably integrated into the potato genome. The pSIM1678 DNA insert or a functional part thereof, is the only genetic material of vector pSIM1678 that is integrated in the potato plant varieties of the invention.

The pSIM1678 DNA insert is described in FIG. 3 (along with vector backbone region), FIG. 5, and Table 4 below. In Table 4, the LB and RB sequences (25-bp each) were synthetically designed to be similar to and function like T-DNA borders from Agrobacterium tumefaciens. GenBank Accession AY566555 was revised to clarify the sources of DNA for the Border regions.

Plasmid pSIM1678 T-DNA is from 1-bp to 9,090-bp and contains two expression cassettes (FIG. 3):

The first cassette (elements 4 to 6, Table 4) contains the 2,626 bp Rpi-vnt1 (Vnt1) gene originating from Solanum venturii. The gene product, VNT1, is an R-protein involved in the plant immune response that protects potato from late blight infection from Phytophthora infestans. The gene is expressed under the native Vnt1 promoter, pVnt1, and terminator, tVnt1.

The second cassette (elements 8 to 14, Table 4) results in down-regulation of vaculor Invertase (VInv) in the transformed potato variety. It is comprised of two fragments of VInv (elements 10 and 12, Table 4) arranged as inverted repeats separated. VInv fragments are arranged between the two convergent potato promoters; the Agp promoter of the ADP glucose pyrophosphorylase gene (Agp) and the Gbss promoter of the granule-bound starch synthase gene (Gbss) that are primarily active in tubers. These promoters drive expression of the inverted repeats to generate double-stranded RNA and down-regulate VInv.

TABLE 4
Genetic Elements of pSIM1678 T-DNA, from Left Border Site to Right Border

| | | Accession | Position | Size | |
|---|---|---|---|---|---|
| Genetic Element | Origin | Number | (pSIM1678) | (bp) | Intended Function |
| 1. Left Border (LB) | Synthetic | AY566555³ | 1-25 | 25 | Site for secondary cleavage |
| site¹ | | (bases 1-25) | | | to release single-stranded |
| | | | | | DNA insert from pSIM1678 |
| 2. Left Border region | S. tuberosum | AY566555³ | 26-187 | 162 | Supports secondary |
| sequence including LB | var. | (bases 1-187) | | | cleavage at LB |
| | Ranger Russet. | | | | |
| 3. Intervening Sequence | S. tuberosum | AF393847 | 188-193 | 6 | Sequence used for DNA |
| | | | | | cloning |
| 4. Native promoter for | S. venturii | FJ423044 | 194-902 | 709 | Drives expression of late |
| the late blight resistance | | | | | blight resistance gene vnt1 |
| gene (Vnt1) | | | | | |
| 5. Late blight resistance | S. venturii | FJ423044 | 903-3,578 | 2676 | Solanum venturii late blight |
| gene VNt1 (Rpi-vnt1) | | | | | resistance protein gene |
| 6. Native terminator for | S. venturii | FJ423044 | 3,579-4,503 | 925 | Ends transcription of late |
| the Vnt1 gene | | | | | blight resistance gene vnt1 |
| 7. Intervening Sequence | S. tuberosumt | HM363755 | 4,504-4,510 | 7 | Sequence used for DNA |
| | | | | | cloning |
| 8. Promoter for the ADP | S. tuberosum | HM363752 | 4,511-6,770 | 2260 | One of the two convergent |
| glucose pyrophosphorylase | var. | | | | promoters that drives |
| gene (pAgp) | Ranger Russet | | | | expression of an inverted |
| | | | | | repeat containing fragments |
| | | | | | of acid invertase gene |
| 9. Intervening Sequence | S. tuberosum | DQ206630 | 6,771-6,776 | 6 | Sequence used for DNA |
| | var. | | | | cloning |
| | Ranger Russet | | | | |
| 10. Fragment of the acid | S. tuberosum | DQ478950 | 6,777-7,455 | 679 | Generates with (12) double |
| invertase (Inv) (sense | var. | | | | stranded RNA that triggers |
| orientation) | Ranger Russet | | | | the degradation of invertase |
| | | | | | transcripts |
| 11. Intervening Sequence | S. tuberosum | X73477 | 7,456-7,461 | 6 | Sequence used for DNA |
| | var. | | | | cloning |
| | Ranger Russet | | | | |
| 12. Fragment of the acid | S. tuberosum | DQ478950 | 7,462-7,965 | 504 | Generates with (10) double |
| invertase (Inv) (anti- | var. | | | | stranded RNA that triggers |
| sense orientation) | Ranger Russet | | | | the degradation of invertase |
| | | | | | transcripts |
| 13. Intervening Sequence | S. tuberosum | X95996 | 7,966-7,971 | 6 | Sequence used for DNA |
| | var. | | | | cloning |
| | Ranger Russet | | | | |
| 14. Promoter for the | S. tuberosum | X83220² | 7,972-8,895 | 924 | One of the two convergent |
| granule-bound starch | var. | | | | promoters that drives |
| synthase (pGbss) gene | Ranger Russet | | | | expression of an inverted |
| (convergent orientation | | | | | repeat containing fragments |
| relative to the pAgp) | | | | | of invertase gene, especially |
| | | | | | in tubers |
| 15. Intervening Sequence | S. tuberosum | AF143202 | 8,896-8904 | 9 | Sequence used for DNA |
| | | | | | cloning |
| 16. Right Border region | S. tuberosum | AY566555³ | 8905-9,065 | 161 | Supports primary cleavage |
| sequence including RB | var. | (bases 231-416) | | | at RB-Like site |
| | Ranger Russet | | | | |
| 17. Right Border (RB) | Synthetic | AY566555³ | 9,066-9,090 | 25 | Site for primary cleavage to |
| sequence¹ | | (bases 392-416) | | | release single stranded DNA |
| | | | | | insert from pSIM1678 (van |
| | | | | | Haaren et al., 1989) |

¹The LB and RB sequences (25-bp each) were synthetically designed to be similar to and function like T-DNA borders from Agrobacterium tumefaciens.
²GenBank Accession HM363755 is replaced with a citation to GenBank Accession X83220 to properly include the full pGbss (2nd copy) DNA insert sequence present in the pSIM1278 construct.
³GenBank Accession AY566555 was revised to clarify the sources of DNA for the Border regions.

Thus, as can be seen from Table 3 and Table 4, the pSIM1678 plasmid is a binary vector designed for potato plant transformation. The vector backbone contains sequences for replication in both E. coli and Agrobacterium along with an ipt marker for screening to eliminate plants with vector backbone DNA. The T-DNA region consists of two expression cassettes flanked by LB and RB sequences. Upon inoculation of host plant tissue with Agrobacterium harboring the pSIM1678 vector, the T-DNA region of pSIM1678 is transferred into the host genome.

### Example 5

### The Agrobacterium Strain and Transfection

The C58-derived Agrobacterium strain AGL1 was developed by precisely deleting the transfer DNA of the hyper-virulent plasmid pTiBo542 (Lazo et al., 1991). A transposon insertion in the general recombination gene (recA) stabilizes recombinant plasmid vectors such as pSIM1278 (FIG. 1). AGL1 displays resistance against carbenicillin and rifampicin, and is eliminated from transformed potato tissue using timentin. Following selection, plants are both antibiotic and Agrobacterium free, with the potato-derived expression cassettes inserted into the plant's genome.

Stock plants were maintained in magenta boxes with 40 ml half-strength M516 (Phytotechnology) medium containing 3% sucrose and 2 g/l gelrite (propagation medium). Potato internode segments of four to six mm were cut from four-week old plants, infected with the Agrobacterium AGL1 strain carrying pSIM1278, and transferred to tissue culture media containing 3% sucrose and 6 g/l agar (co-cultivation medium). Infected explants were transferred, after two days, to M404 (Phytotechnology) medium containing 3% sucrose, 6 g/l agar and 150 mg/l timentin to eliminate Agrobacterium (hormone-free medium). Details of the methods are described in Richael et al. (2008).

After one month, the infected explants were transferred to fresh medium lacking any synthetic hormones and incubated in a Percival growth chamber under a 16 hr photoperiod at 24° C. where they started to form shoots. Many shoots expressed the ipt gene and displayed a cytokinin overproduction phenotype; these shoots were not considered for further analyses. PCR genotyping demonstrated that about 0.3 to 1.5% of the remaining shoots contained at least part of the P-DNA while lacking the ipt gene. Thus, no markers were used to select for the transformed plants. Details on ipt-based marker-free plant transformation were published by Richael et al. (2008).

The process of eliminating Agrobacterium started two days after explant infection. For this purpose, tissues were subjected to the antibiotic timentin (150 mg/L) until proven to be free of live Agrobacterium. Proof was obtained by incubating stem fragments of transformed events on nutrient broth-yeast extract (NBY medium) for 2 weeks at 28° C. (repeated twice). In accordance with 97 CFR Part 340, transformed plants were transported and planted in the field only when free of live Agrobacterium.

The Russet Burbank W8 event contains inserts derived from two separate transformations with different plasmids. The first insert, plasmid pSIM1278, contains two cassettes consisting of inverted repeats designed to silence up to four potato genes, Asn1, Ppo5, R1, and PhL, in tubers. Similarly, the second plasmid, pSIM1678, contains a cassette consisting of an inverted repeat to silence the VInv gene in tubers, while also containing a copy of the Rpi-vnt1 gene under its native potato promoter.

Potato plant varieties were analyzed by DNA gel blot analyses to determine the structure and copy number of integrated DNA insert sequences and to confirm the absence of vector backbone sequences.

In addition, molecular characterization was used to determine the sequence of the junctions flanking the DNA insert and show stability of the inserted DNA.

Sequencing information of the junctions provided a basis for developing specific PCR tests for the intragenic potato plant varieties. Thus, the disclosed methods are broadly applicable to other plant species, and other potato cultivars, as it is within the skill level of an artisan in this field to sequence the DNA of a plant species that has undergone a transformation event and identify the non-naturally occurring nucleotide junctions resultant therefrom. Said artisan would then be able to develop an appropriate probe that binds to said non-naturally occurring junction sequences and primers optimized to amplify such sequence.

### Example 6

### Evidence for the Absence of the Vector Backbone DNA

Unlike many commercial transgenic crops, potato cultivars of the disclosure were confirmed to be free of Agrobacterium-derived DNA sequences that are used for transformation, such as vector backbone DNA, by three different methods: 1) First, the presence or absence of the negative selectable isopentenyl isomerase (ipt) marker gene in the vector backbone was determined, as inadvertent transfer of backbone DNA comprising the ipt gene expression cassette from Agrobacterium to plant cells would trigger ipt gene expression and, consequently, the formation of the cytokinin-type hormone isopentenyladenosine, 2) Southern blot hybridization was then used on the transformed potato plants that had passed the first screening method to confirm the absence of backbone DNA, and 3) PCR was then designed to amplify fragments indicative of junctions between DNA insert border regions and flanking backbone DNA or regions within the backbone DNA that flank the DNA insert. The efficacy of the method was confirmed by using pSIM1278 DNA as a positive control. Potato cultivars of the present disclosure did not produce PCR bands indicative of the presence of vector backbone DNA.

### Example 7

### Stability of the Inserted DNA

The stability of DNA inserts was evaluated in the original transformants and again in propagated plant material using both DNA gel blot hybridization and trait evaluation. These studies were carried out to ensure that intragenic events expressed the incorporated traits in a consistent and reliable manner. Instability might be triggered by rare recombination events or could also be caused by methylation. Because potatoes are normally propagated clonally, standard assessments for sexually propagated crops were not directly applicable, and tubers rather than seeds were used to define subsequent generations. Results of DNA blot hybridization demonstrate consistent bands were present in multiple generations, thus indicating stability. Further evidence for stability was obtained by confirming trait efficacy in generations one and two tuber seed.

DNA insert stability was demonstrated in the originally-transformed material (G0) by extracting and evaluating DNA from leaves of plants that had been propagated in vitro and never planted in soil. For generation-1 (G1) analyses, two propagated plants from each intragenic variety and one plant from each control were planted in the greenhouse; one of the tubers harvested from each plant was planted to obtain leaves from G1 plants that were used to isolate DNA and evaluate the G1 generation. Tubers from this generation were planted again, and leaves of the resulting G2 plants allowed a characterization of that generation.

The structure of the insert was shown to be stable using Southern blot analysis of genomic DNA isolated over three generations of W8 potatoes (G0-G3), whereas the phenotypic stability was assessed by measuring polyphenol oxidase activity, in the second generation of field-grown tubers. This method shows visual evidence of PPO silencing after applying catechol to the cut surface of potatoes. These studies were carried out to ensure that the desired genetic changes in W8 remained stable over multiple clonal cycles while maintaining the traits.

The stability of the DNA inserts was evaluated by comparing three successive clonal generations (G1, G2, and G3) to the original transformant (G0) using Southern blots. Stable DNA inserts are expected to maintain the same structure and thus produce the same digestion patterns over multiple generations of the plant. To test stability of the inserts in the W8 event, its digestion pattern was compared using two probes (GBS1 and AGP) that hybridize to regions of the inserts from both pSIM1278 and pSIM1678, and two probes (INV and VNT1) that are specific to the pSIM1678 insert. Since the DNA sequences these probes hybridize with are contained in the potato genome as well as within the DNA insert(s), both endogenous and insert-specific bands are expected in the Southern blots.

All genomic DNA samples were digested with the restriction enzyme, EcoRV, and hybridized with a probe specific to either AGP or GBS1. EcoRV was chosen for these studies as it digests within both inserts to provide a unique banding pattern with internal bands of predicted size in the pSIM1278 insert (e.g. 2.3 kb). The banding patterns between all samples of W8 were identical to each other for both probes. The multiple bands present in the Russet Burbank control are also found in W8, but W8 also contains bands corresponding to the pSIM1278 and pSIM1678 inserts. These bands are similarly consistent between all generations of W8 analyzed indicating genetic stability of both inserts.

A second analysis was performed using two probes specific to the pSIM1678 insert. For this analysis, genomic DNA samples were digested with the restriction enzyme, XbaI, and hybridized with VNT1 and INV probes. XbaI was chosen as the restriction enzyme for these studies as it digests the pSIM1678 internally and produces a band of known size (e.g. 4.6 kb for the INV probe). Again, both endogenous and insert-specific bands were detected with consistent banding patterns between the three generations analyzed. The genetic and phenotypic analyses indicated the insertions arising from transformation of both pSIM1278 and pSIM1678 are stable over three generations. Given the demonstrated stability over three generations, it is likely that stability will be maintained during subsequent cycles of vegetative propagation.

### Example 8

### Junction Analysis and Variety-Specific Detection

DNA insert/flanking plant DNA junctions were sequenced using either Adapter Ligation-Mediated PCR or Thermal Asymmetric Interlaced PCR.

The junction sequences were used to design primers for potato cultivars of the disclosure, and these primers were applied for variety-specific PCR-based detection methods.

Primers can be used to amplify a variety-specific DNA fragment, resulting in a line specific test method for said variety. The methods developed were used to monitor plants and tubers in field and storage to confirm the absence of intragenic material in tubers or processed food, and to ensure the purity of organic seed.

### Example 9

### Efficacy and Tissue-Specificity of Gene Silencing

Gene silencing methods were employed to lower the activity of the Asn1, Ppo5, PhL, R1 and VInv native proteins, and transcript levels rather than protein amounts were evaluated to link new phenotypic traits to changes at the molecular level.

Since strong silencing of the Asn1 gene involved in ASN (asparagine) formation in leaves and stems might adversely affect growth, the Agp promoter and the Gbss promoter, which are tuber- and stolon-specific promoters and are much less active in photosynthetically-active tissues and roots, were used to drive gene silencing in tubers and stolons. The transcript levels of the five targeted genes in various tissues of plant varieties, along with their untransformed counterparts were determined by Northern blot analysis.

Two of the three gene silencing cassettes introduced into Russet Burbank to generate the W8 event were very effective at silencing their target transcripts for RNAi-mediated silencing. These two constructs effectively silenced Asn1, Ppo5, and VInv in the tubers of W8. The specificity of silencing to the tubers indicates that few, if any, of the siRNA generated by the RNAi machinery spread to other tissues or that their levels were insufficient to invoke an RNAi response in those tissues. The only evidence for silencing outside of tubers was in flowers where lower levels of Asn1 were observed, yet the magnitude of change was much lower than in tubers. The promoter silencing strategy with PhL and R1 had minimal effect, which was consistent with other events containing the same pSIM1278 construct (Collinge and Clark 2013).

A summary of the down-regulated transcript levels in specific tissues of several intragenic potato cultivars is shown in Table 5. Each letter (A, P, L, R) in Table 5 indicates that silencing was confirmed, although the amount of silencing varied depending on the gene and tissue.

TABLE 5
Summary of Down-Regulated Genes in Different Tissues

| Event | Tubers¹ | | | | Stolons¹ | | | Roots¹ | | | Stems¹ | | Leaves¹ | Flowers^{1,2} |
|---|---|---|---|---|---|---|---|---|---|---|---|---|---|---|
| F10 | A | P | | R | A | L | R | A | P | R | | P | A | A |
| E12 | A | P | L | R | A | L | R | A | P | | | P | | A |
| J3 | A | P | L | R | A | | R | A | | | A | P | | A |
| J55 | A | P | L | R | A | L | R | A | | | | P | | A |

¹A = Asn1, P = Ppo5, L = PhL, R = R1. Letters in table indicate down-regulated gene expression by tissue.
²The partially down-regulated Asnl gene expression might alter the amino acid composition of the flowers. Such effects will be limited to a reduction in ASN and an increase in GLN. Since ASN and GLN are similar non-essential amino acids, changes in the levels of these compounds is not expected to affect the quality of petal, nectar, and pollen as feed for insects or other organisms.

### Example 10

### DNA Isolation from Potato Plant Leaf Tissue

DNA was extracted from 3 g of potato plant leaf tissue, ground in liquid nitrogen and mixed with 20 ml of Extraction Buffer (0.35 M Sorbitol, 0.1 M Tris-HCl, pH 8.0, 0.5 M EDTA, pH 8.0). Samples were pelleted at 3,000 rpm for 5 min and rinsed with 2 ml Extraction Buffer. Pellets were resuspended in 4 ml of Extraction Buffer and 4 μl of 100 mg/ml RNase A. Four milliliters of Nuclear Lysis Buffer (1 M Tris-HCl, pH 8.0, 0.5 M EDTA, pH 8.0, 5 M NaCl, 20 mg/ml CTAB) and 1.6 ml of 5% Sarcosyl were added to each sample. Samples were mixed and incubated at 65° C. for 20 min with agitation. Samples were shaken with an equal volume of chloroform:isoamyl alcohol (24:1) and centrifuged at 3,000 rpm for 5 min. The aqueous phase was kept and the chloroform extraction step was repeated 2-3 times. DNA was precipitated in an equal volume of isopropyl alcohol and pelleted at 3,000 rpm for 10 min. Pellets were rinsed in 70% ethanol, dried and resuspended in TE.

### Example 11

### High-Yield CTAB-Based DNA Extraction Method

Potatoes and potato products contain high levels of polysaccharides that can interfere with PCR, particularly qPCR. Thus, it is recommended that DNA isolation is performed with a method that yields high quality DNA and that qPCR is performed using master mixes designed to prevent interference by PCR inhibitors, such as polysaccharides. To satisfy these requirements, a cetyltrimethylammonium bromide (CTAB) isolation method and the PerfeCTa® qPCR ToughMix® available from Quanta (Example 16) are used. These PCR methods using event-specific primers tested on DNA extracted from potato leaf material have resulted in high PCR efficiency, good linearity, target specificity, and robustness.

### Protocol for Extraction Method

1. Make CTAB buffer fresh daily (1M Tris-HCl, pH 8.0, 0.5 M EDTA pH 8.0, 5M NaCl and 20 mg/ml CTAB) and pre-warm to 65° C.
2. Add 10 mL CTAB to chip and tuber/30 mL CTAB Buffer to flake and fry.
Add 1 mL CTAB to leaf.
3. Add the following amount of starting material to an appropriate tube:
Flake: 3 g
Freeze-dried Fry: 3 g (if using fresh fries use 6 g)
Chip (grind chips to a smooth paste): 1 g
Freeze-dried Tuber: 1 g
Freeze-dried Leaf (grind to a fine powder): 0.5 g
4. Add 3 μL per ml of Proteinase K to each tube and mix to remove clumps.
5. Incubate at 65° C. with shaking in incubator at 210 rpm for 2-3 hours (45′ is sufficient for tuber and leaf)
6. Centrifuge samples at 14,000 rpm for 40 minutes (20′ for tuber and 10′ for leaf is sufficient)
7. Transfer supernatant to a clean tube and incubate on ice for 20 minutes
8. Add an equal volume of ice-cold chloroform
9. Optional: Add 200 μL, of PhytoPure DNA extraction resin (GE Healthcare Life Sciences) for every 1 g of starting material. Vortex resin before use to ensure homogenous.
10. Mix samples vigorously at room temperature.
11. Centrifuge samples at 14,000 rpm for 40′ (20′ for tuber and 10′ for leaf is fine)
12. Transfer upper aqueous phase to a clean tube.
13. Repeat chloroform extraction until clean interface (no additional resin required).
14. Add 1/10th volume of 3M Na-Acetate (pH 5.3)
15. Add an equal volume of isopropanol.
16. Invert tube until DNA precipitates (overnight at 4° C. is optimal)
17. Centrifuge at 14,000 rpm for 20′ to pellet DNA (5′ is sufficient for leaf). At this point some samples may not have an obvious pellet. In this case, carefully pipet off the upper clear layer and leave the lower darker layer, this will pellet during step 19.
18. Wash pellet with ice-cold 70% ethanol.
19. Centrifuge pellet at 14,000 rpm for 5-10 minutes.
20. Air-dry pellet for 10′ or until pellet edges look clear.
21. Resuspend DNA in 200-400 μL TE buffer.
22. Add RNase to a concentration of 20 μg/ml. Incubate at 37° C. for 30 minutes.
23. Add 5 volumes of Qiagen Buffer PB to DNA. Do not exceed 10 μg of DNA per column.
24. Spin DNA/PB solution through the column.
25. Wash 2 times with buffer AW2 or PE
26. Spin column at 14,000 rpm for 2 minutes to dry column.
27. Apply appropriate amount of TE (50 μL) to column and let sit at 65° C. for 5′ before eluting.
28. DNA concentration should be measured using a fluorescent intercalating dye (e.g. Qubit High-sensitivity) and samples should be tested for PCR inhibitors through serial dilution analysis, particularly if performing quantitative analyses.

### Example 12

### DNA Isolation from Tuber, Flake, Chip, and Fry Using the QIAamp Fast DNA Stool Mini Kit

The following protocol was conducted to isolate DNA. DNA samples isolated using this technique tend to be less pure and of lower yield than the CTAB-based method described in the preceding section. Care must be taken to ensure the DNA is of sufficient quality for any quantitative analysis due to the presence of PCR inhibitors. Any matrix-specific instructions are bolded.

Weigh approximately 300 mg of flake, chip, or fry tissue and place into a 2.0 ml microcentrifuge tube containing 1.7 ml Qiagen InhibitEX Buffer (preheat buffer to redissolve any precipitates). For freeze-dried tuber use 150 mg tissue in 1.2 ml InhibitEX Buffer. Fry and flake material will require two 300 mg samples to be lysed separately and combined over a single column downstream. For chip samples, optionally two samples can be lysed and combined to increase yield.

Mix thoroughly by vortexing for 30-60 seconds.

Incubate flake, chip, and tuber samples in a heated block (preferred) or water bath at 95° C.±5° C. for 30 min. For fry samples, incubate instead at 70° C. for 30 min.

Centrifuge tubes in a microcentrifuge for 5 min at 14,000 rpm.

Transfer 600-650 μl supernatant from each tube into individual 2.0 ml microcentrifuge tubes, and add 25 μl Proteinase K provided in the Qiagen Kit.

Mix thoroughly by vortexing.

Add 600-650 μl Buffer AL to each tube and mix by vortexing.

Incubate samples for 10 min at 70° C.

Add 600-650 μl 95-100% ethanol (not provided in kit) and mix by vortexing.

Pass solution 650 μl at a time through a QiaAmp Spin Column using a vacuum manifold or pulse centrifugation at 21,000×g. For fry and flake material, pass two tubes worth of solution over a single column to concentrate. The final spin should be 1 min at 14,000 rpm to remove all buffer.

Wash columns once with 500 μl Buffer AW1 and once with 500 μl of Buffer AW2 using a vacuum manifold or centrifugation at 14,000 rpm for 1 min.

Move columns to 2 ml collection tubes, centrifuge for 3 min at 14,000 rpm and discard flow-through.

Transfer column to a new 1.7 ml microcentrifuge tube and pipette 50 μl of TE for tuber and chip or 30 μl TI for fry and flake samples directly onto the membrane.

Incubate for 3 min at room temperature.

Centrifuge for 1 min at 14,000 rpm to elute DNA.

Quantify and QA the DNA (store at −20° C.). The (OD₂₆₀/OD₂₈₀) and (OD₂₃₀/OD₂₆₀) ratios should be documented for all samples. DNA concentration should be measured using a fluorescent intercalating dye (e.g. Qubit dsDNA HS Assay Kit from Life Technologies) and samples should be tested for PCR inhibitors through serial dilution analysis, particularly if performing quantitative analyses.

### Example 13

### Molecular Characterization of DNA Inserts and Non-Naturally Occurring Junction Sequences in Potato Events

All potato events were analyzed by DNA gel blot analyses to determine the structure and copy number of the integrated DNA insert sequences and to confirm the absence of vector backbone sequences. These studies were carried out as part of the characterization and biosafety assessment of the events. Molecular characterization was used to determine the sequence of the junctions flanking the DNA insert and show stability of the inserted DNA. Sequencing information of the junctions provided the basis for developing event-specific PCR tests for all events (Table 6).

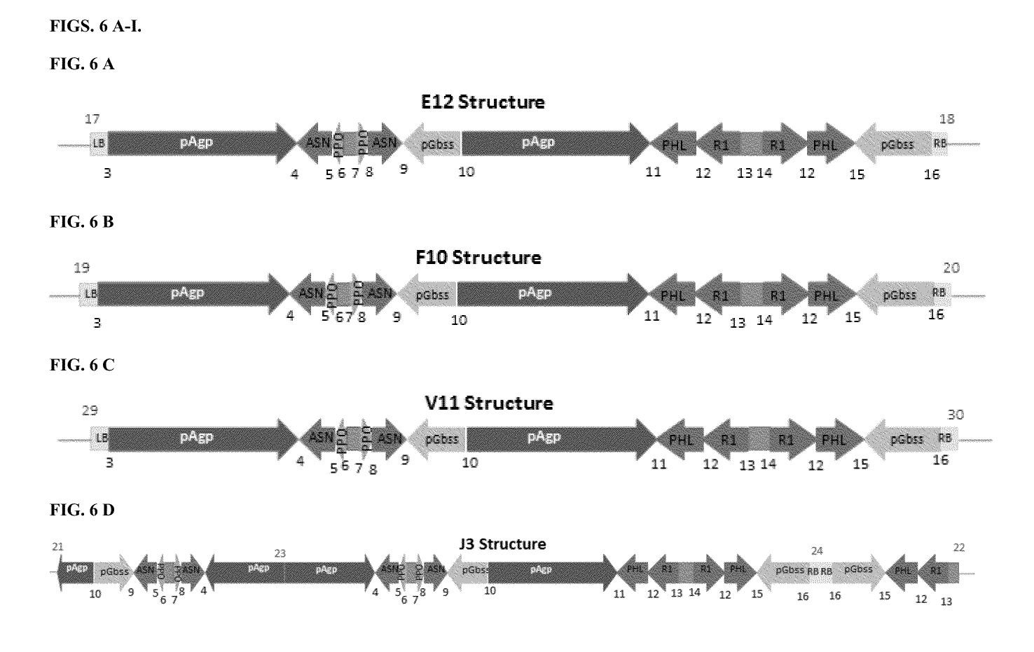

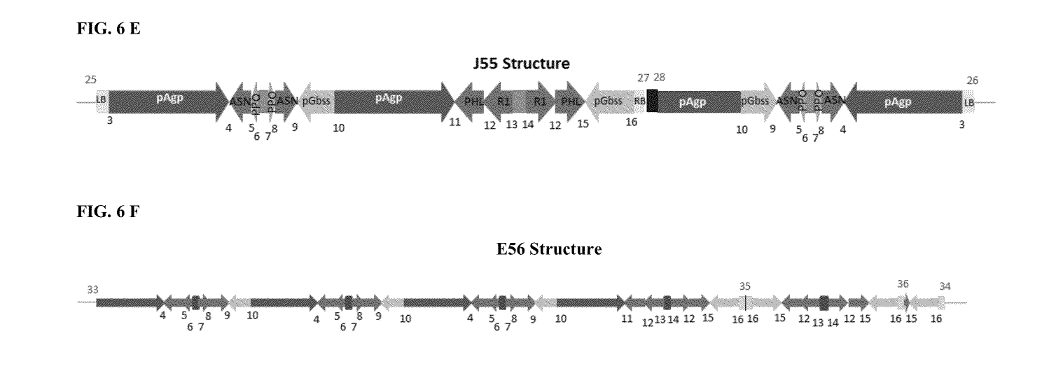

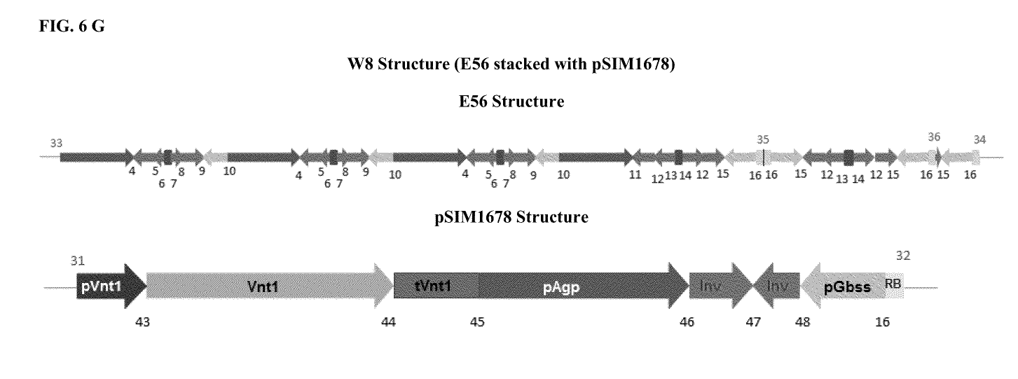

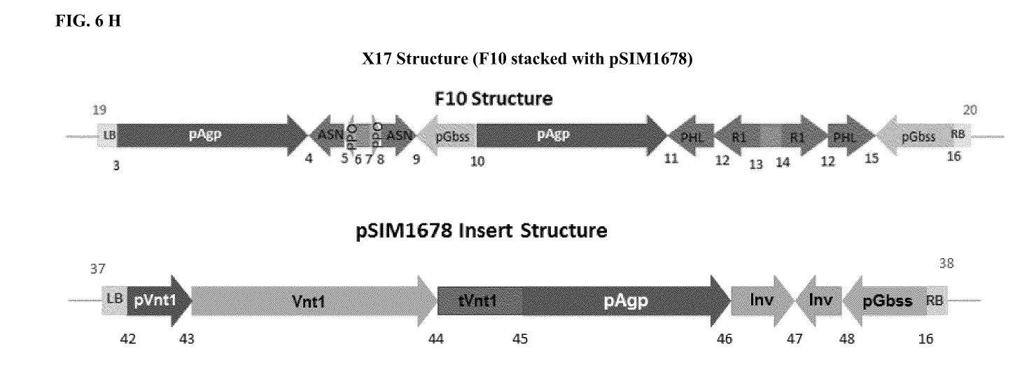

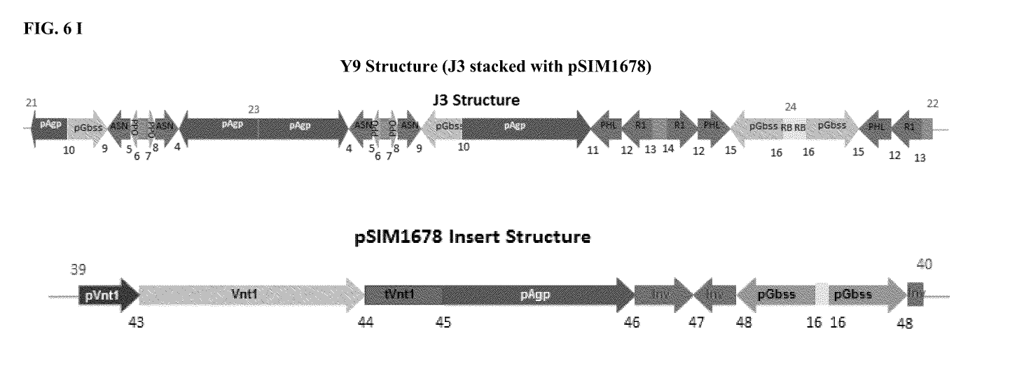

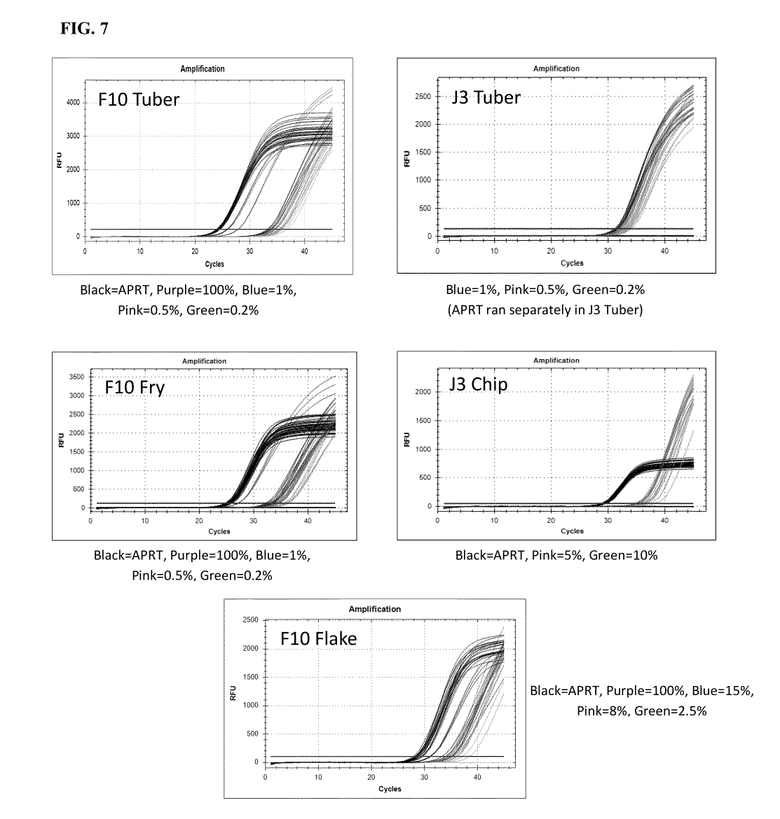

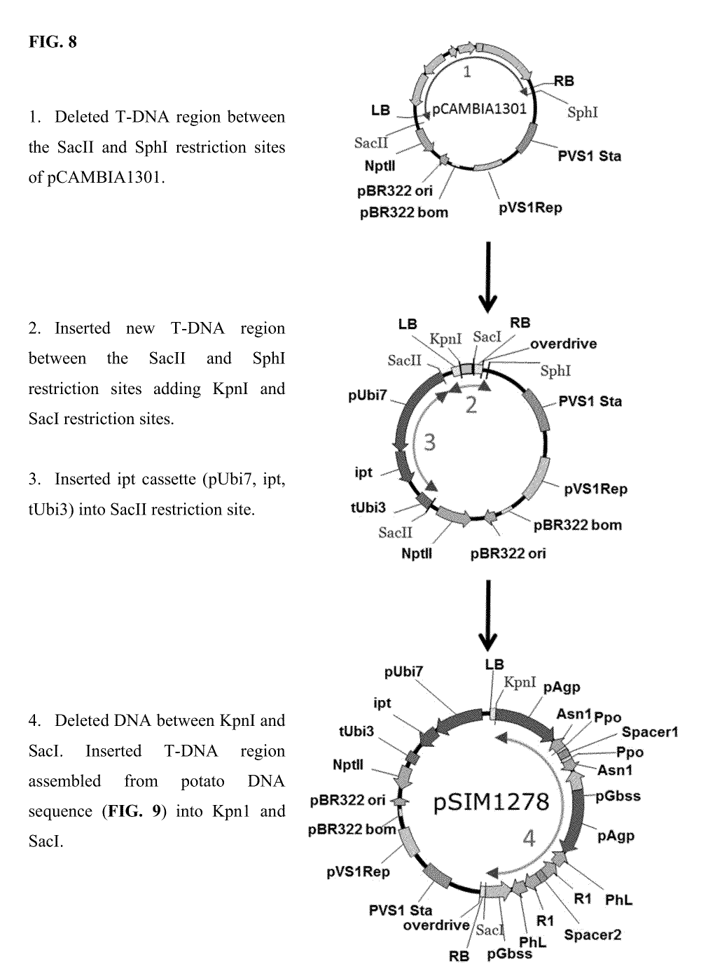

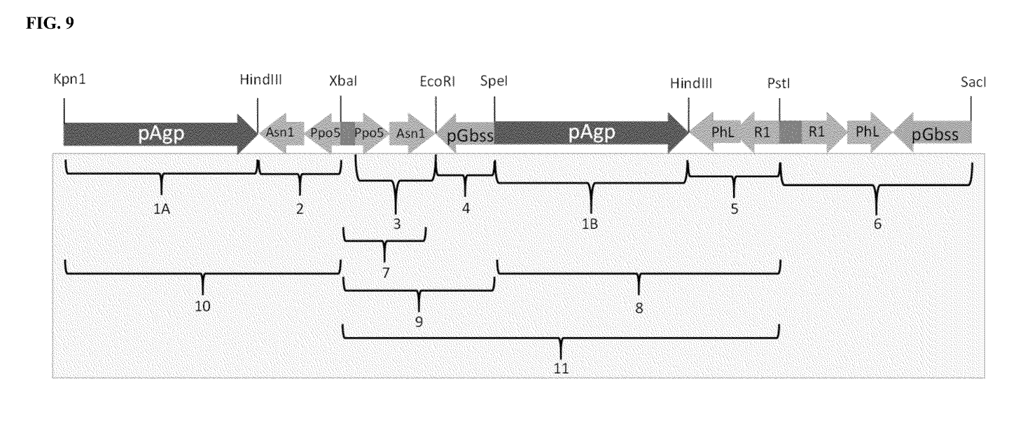

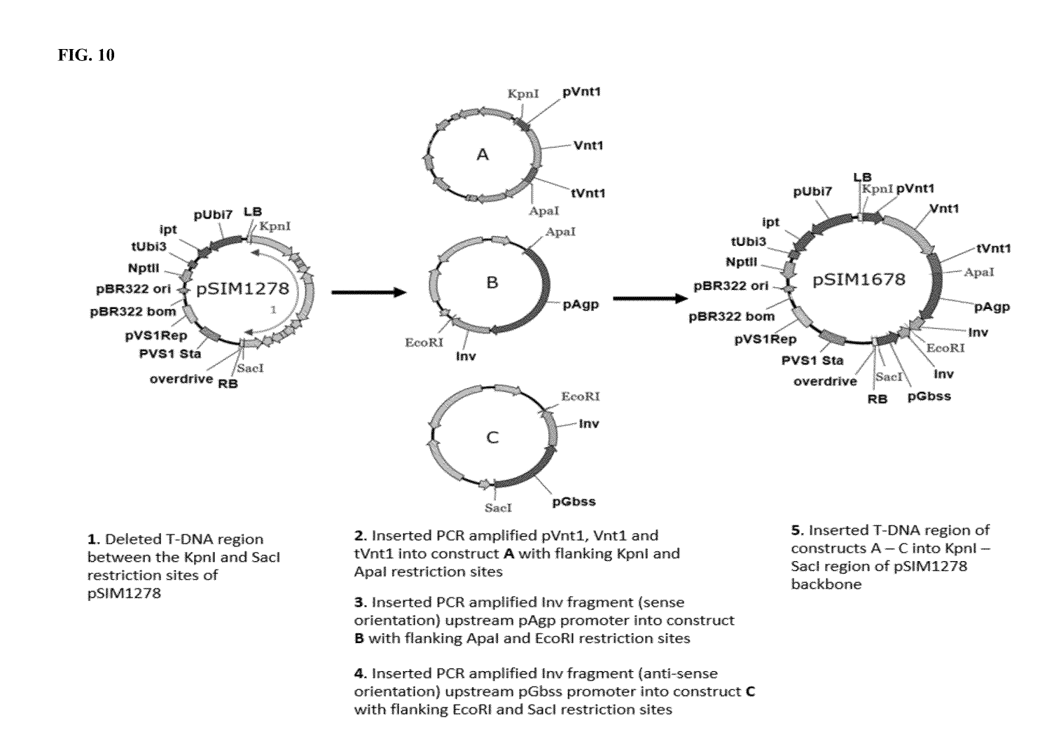

FIG. 5 shows with SEQ ID NOs from Table 6 where each construct-specific non-naturally occurring junction occurs for the DNA insert regions of pSIM1278 and pSIM1678, and FIG. 6 A-I shows with SEQ ID NOs from Table 6 where each construct-specific and event-specific non-naturally occurring junction occurs for E12, F10, J3, J55, V11, W8, X17, and Y9 events

With respect to Table 6, the first two sequences (SEQ ID NOs: 1 and 2) represent junctions that exist on the far outside edge of the insert that are 25 nucleotides of synthetic sequences that are not part of the potato genome. They exist on both the left border and right border. These are included in Table 6 as LB synthetic/LB potato and RB synthetic/RB potato

Further, Table 6 provides in bold face and highlighted type the: 1) Sequence micro-homologies to chromosomal DNA, which is where the sequence is common to both chromosomal DNA and to border sequences for inserts. These are listed in bold and highlighted type within the sequences where they exist. Table 6 also provides: 2) Intervening sequences that exist between the junction sites. These sequences are underlined and there has been left 15 bp of sequence on either side of the junction.

TABLE 6
Construct-specific and Event-specific Non-naturally Occurring Junction Sequences

| | | |
|---|---|---|
| | | SEQ ID |
| Junction | Sequence | NO. |

pSIM1278 construct junctions

| | | |
|---|---|---|
| LB Synthetic/LB | 5′-TGGCAGGATATATACCGGTGTAAAC/GAAGTGTGTGTGGTT-3′ | 1 |
| Potato | | |
| RB Synthetic/RB | 5′-GTTTACAGTACCATATATCCTGTCA/GAGGTATAGAGGCAT-3′ | 2 |
| Potato | | |
| LB/AGP | 5′-TTGTGGAGGAGTAAG/GGTACC/AAGTGTCTGAGACAA-3′ | 3 |
| AGP/ASN | 5′-AACAAGCTTGTTAAC/GAACTCTTTATCCAG-3′ | 4 |
| ASN/PPO | 5′-TGGTGAGCCCTCGAG/ATAATTGTAACTGAT-3′ | 5 |
| PPO/Spacer1 | 5′-ATTCAATAGAGACTA/TCTAGA/GTGTATGGGTGATCC-3′ | 6 |
| Spacer1/PPO | 5′-TTATGGAGGAATCAG/TAGTCTCTATTGAAT-3′ | 7 |
| PPO/ASN | 5′-ATCAGTTACAATTAT/TCTCGAGGGCTCACC-3′ | 8 |
| ASN/GBSS | 5′-CTGGATAAAGAGTTC/GAATTC/GTGATGTGTGGTCTA-3′ | 9 |
| GBSS/AGP | 5′-TGAGATGCATGGTTC/ACTAGTGATTGGTACC/AAGTGTCTGAGACAA-3′ | 10 |
| AGP/PHL | 5′-AACAAGCTTGTTAAC/GTGCTCTCTATGCAA-3′ | 11 |
| PHL/R1 | 5′-TTTTGCTAAAACATC/GGACACGTATATTTT-3′ | 12 |
| R1/Spacer2 | 5′-ATATTTATTATATAA/CTGCAG/CGTTGTTTTGATGAA-3′ | 13 |
| Spacer2/R1 | 5′-TTTTGATGAAAAAGC/TTATATAATAAATAT-3′ | 14 |
| PHL/GBSS | 5′-TTGCATAGAGAGCAC/CGTGATGTGTGGTCT-3′ | 15 |
| GBSS/RB | 5′-AAATCATAATATCTG/GAGCTCATC/GTTATGCTATAAATT-3′ | 16 |

E12 specific junctions

| | | |
|---|---|---|
| (LJ-plant/insert) | 5′-TATTGGTGAGAATAA/TAAACGAAGTGTGTG-3′ | 17 |
| (RJ-insert/plant) | 5′-ACGGTCAGTCACTTT/GTACTAGTAAAGATC-3′ | 18 |

F10 specific junctions

| | | |
|---|---|---|
| (LJ-plant/insert) | 5′-CAGCAGCCATCAACT/GGTCCA/GCAGGATATATACCG-3′ | 19 |
| (RJ-insert/plant) | 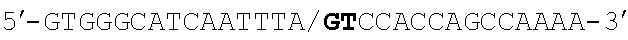 | 20 |

J3 specific junctions

| | | |
|---|---|---|
| (LJ-plant/insert) | 5′-AACAGGACAACCACA/AGCTA/GGAAACTCACATGCT-3′ | 21 |
| (RJ-insert/plant) | 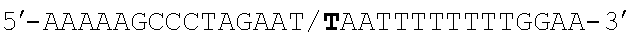 | 22 |
| (internal) | 5′-GAACTGAAACCGATA/TACAAAATGGTATAA-3′ | 23 |
| (internal) | 5′-CCTCTATACCTCTGA/CAGAGGTATAGAGGC-3′ | 24 |

J55 specific junctions

| | | |
|---|---|---|
| (LJ-plant/insert) | 5′-AAATCAACAAGCAAT/TGAAGAACTACCTAT-3′ | 25 |
| (RJ-insert/plant) | 5′-CATAAGTGAGACTAT/GCGTGAAACTTTCAG-3′ | 26 |
| (internal) | 5′-CCTCTATACCTCTGA/TAAATTAAACTGACT-3′ | 27 |
| (internal) | 5′-ACTAAAAATCTCAGC/TAAAACAATAAAAAT-3′ | 28 |

V11 specific junctions

| | | |
|---|---|---|
| (LJ-plant/insert) | 5′-TCATCTTTCATTCCG/GTGTAAACGAAGTGT-3′ | 29 |
| (RJ-insert/plant) | 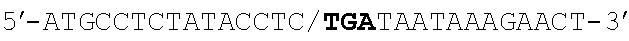 | 30 |

W8 specific junctions

| | | |
|---|---|---|
| (LJ-plant/pSIM1678 | 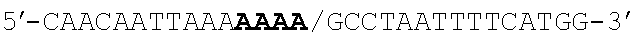 | 31 |
| insert) | | |
| (RJ-pSIM1678 | 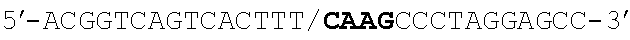 | 32 |
| insert/plant) | | |
| (LJ-plant/pSIM1278 | 5′-AGCCCTACAAAAGGC/CCAAACTCTAAGTCA-3′ | 33 |
| insert) | | |
| (RJ-plant/pSIM1278 | 5′-AGTCCTAGAACTACG/AAAATTATATTCGGT-3′ | 34 |
| insert) | | |
| (internal) | 5′-ATGCCTCTATACCTC/AGAGGTATAGAGGCA-3′ | 35 |
| (internal) | 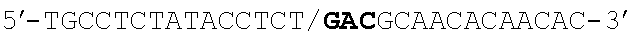 | 36 |

X17 specific junctions

| | | |
|---|---|---|
| (LJ-plant/insert) | 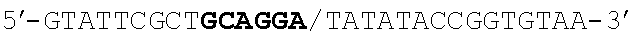 | 37 |
| (RJ-insert/plant) | 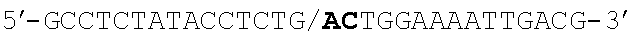 | 38 |

Y9 specific junctions

| | | |
|---|---|---|
| (LJ-plant/insert) | 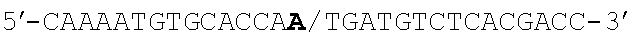 | 39 |
| (RJ-insert/plant) | 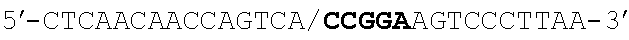 | 40 |
| (internal) | 5′-ATGCCTCTATACCTC/TGAAAGTGACTGACC-3′ | 41 |

pSIM1678 construct junctions

| | | |
|---|---|---|
| LB/pVnt1 | 5′-TTGTGGAGGAGTAAG/GGTACC/AGTTATACACCCTAC-3′ | 42 |
| pVNT1/Vnt1 | 5′-ACCAGCTAACAAAAG/ATGAATTATTGTGTT-3′ | 43 |
| Vnt1/tVnt1 | 5′-TCACAGGTACTATAA/ATAATTATTTACGTT-3′ | 44 |
| tVnt1/pAGP | 5′-AGCCTCTTTTCAAAG/GGGCCC/CAAGTGTCTGAGACA-3′ | 45 |
| pAGP/Inv | 5′-AACAAGCTTGTTAAC/GGATCC/ACATTCCTCCCGGAT-3′ | 46 |
| Inv/Inv | 5′-TCAAGGACTTTAGAG/GAATTC/AGCGGACCCAGTCCA-3′ | 47 |
| Inv/pGbss | 5′-GAGGAATGTGTAATG/ACTAGT/CGTGATGTGTGGTCT-3′ | 48 |

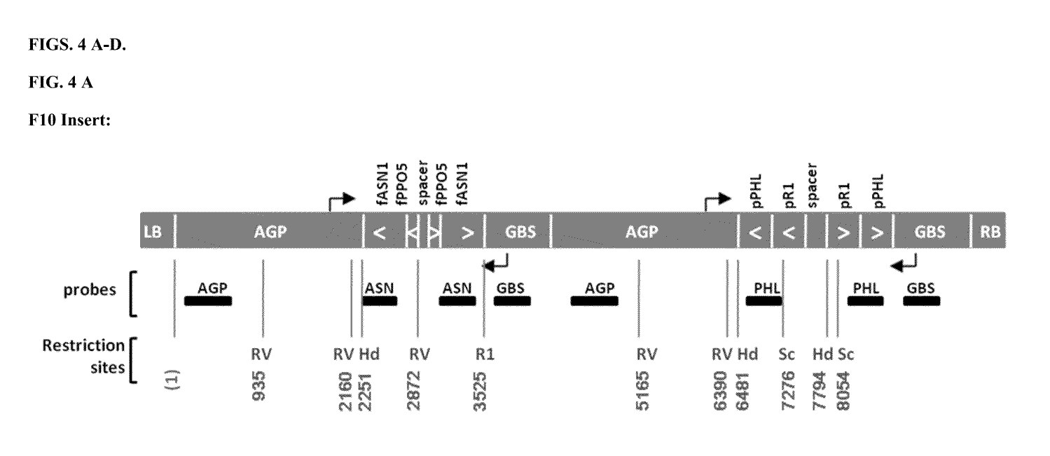

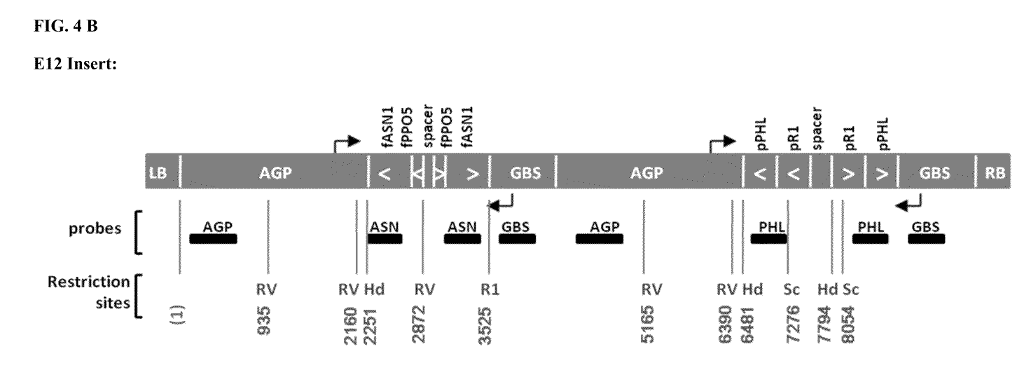

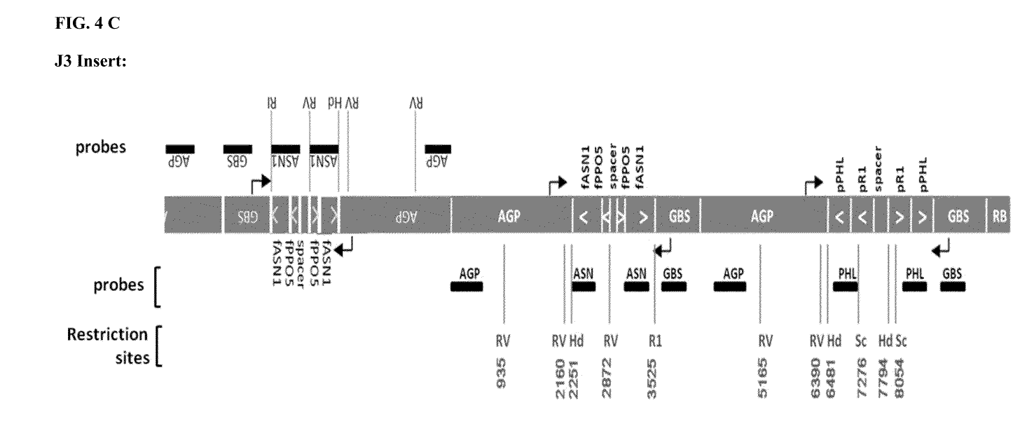

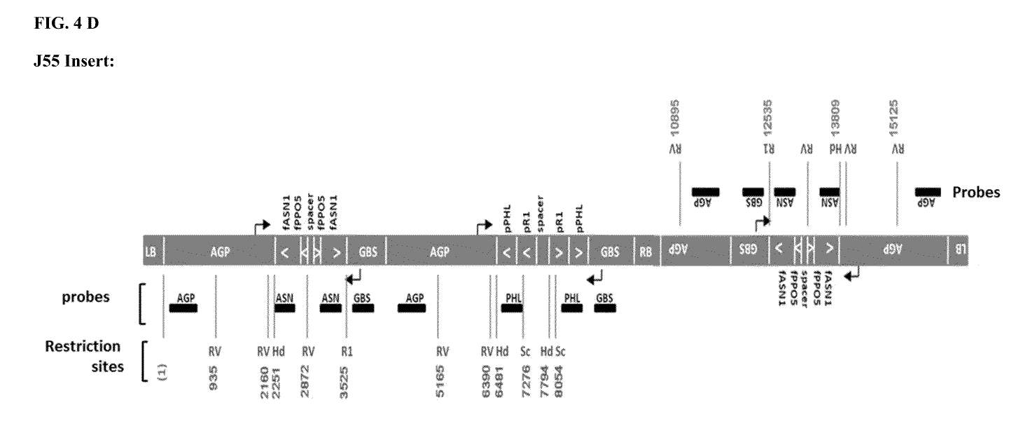

A diagram of the structures of DNA inserts in potato events E12, F10, J3, J55, V11, W8, X17, and Y9 are shown in FIG. 4 and FIG. 6.

Events E12 and F10 contain 1 copy (whereby “copy” implies the presence of at least an Asn1/Ppo5 gene silencing cassette). Events J3 and J55 contain 2 copies. There were no differences in the extent and persistence of silencing activities between higher-copy events and events with only one copy.

Event J55 contained two linked DNA inserts positioned as an inverted repeat (FIG. 4 and FIG. 6). Lechtenberg et al. (2003) showed with bacterial T-DNA that the presence of a second gene copy either in tandem or an inverted arrangement did not result in silencing. Thus, it's likely that the inverted linked DNA insert copies in J55 would not contribute to silencing and therefore, the silencing of targeted genes functions as intended based on the inverted repeats positioned between convergent promoters.

Genetic and structural characterization of the inserts associated with transformation of Russet Burbank by pSIM1278 and pSIM1678 to produce event W8 showed that both transformations resulted in a single integration site for each plasmid. The structure of the DNA derived from transformation of pSIM1278 was complex relative to the structure of the original insert. The inserted DNA appears to have undergone rearrangement during transformation resulting in a structure consisting of a tandem repeat of the Asn1/Ppo5 silencing cassette, followed by a nearly complete pSIM1278 construct, and an inverted repeat containing a duplication of the pR1/pPh1 silencing cassette and a tandem duplication of the Gbss promoter with intervening Ph1 sequence (FIG. 6).

Although this structure is more complicated than anticipated, the duplicated silencing cassettes are intact and remain under the control of the tissue-specific promoters. The structure does not negatively impact safety or trait efficacy of the product.

W8 also contains a single copy of the DNA from pSIM1678 that resides at a single locus of integration (FIG. 6). The DNA insert of pSIM1678 contains a nearly intact DNA insert with a 330-bp deletion, which removes the entire T-DNA left border and 137-bp of the Rpi-vnt1 promoter. This small deletion in the promoter does not affect the gene's ability to confer late blight resistance. Also, RNA expression associated with the Rpi-vnt1 gene has been demonstrated using RT-PCR.

Inserts are occasionally flanked by short DNA sequences that are derived from the plant genome or the DNA insert. These insertions appear to be part of the integration process and occur at rather high frequencies (Windels et al. 2003). An example of an event with such sequences includes the 49-bp sequence between the two DNA inserts of J55. A blast search of this short DNA sequence using GenBank partially matched known sequences from S. tuberosum, confirming that the origin was most likely from either the plant genome or the DNA insert.

Most transferred DNA inserts are shorter than the full distance between the Left and Right Border sequences, as shown by short deletions near the borders. Such deletions are also associated with T-DNA integration and hypothesized to result from double-strand break repair (Gheysen et al. 1991). A short deletion that did not impair the functional activity of the two silencing cassettes was found in, for instance, event F10 (a 38-bp deletion at the Right Border).

### Example 14

### qPCR Primer and Probe Development

Event-specific primers were designed to amplify a region of the genome that is either specific to the event of interest (potato genome flanking region) or to a junction in the pSIM1278 construct or pSIM1678 construct itself. These primers amplify a region of 70 to 200 base pairs within which region binding of a target-specific fluorescent probe allows real-time detection and quantitation of product.

Each fluorescent probe is labeled at the 5′ end with a 6-FAM (6-carboxyfluorescein) moiety and at the 3′ end with a BHQ1 (Black Hole Quenchers™ 1) moiety. Fluorescence by 6-FAM is quenched by the presence of BHQ1 on the same oligonucleotide. During PCR, the probe annealed to the target strand being amplified will be cleaved by the 5′-to-3′ nuclease activity of Taq DNA polymerase, resulting in the separation of the 6-FAM and BHQ1 moieties. The quenching of 6-FAM by BHQ1 is thus abolished, leading to the emission of the 6-FAM fluorescence. The probes are designed as locked nucleic acid (LNA) probes to increase thermal stability and specificity (Petersen, M., & Wengel, J. (2003). LNA: A versatile tool for therapeutics and genomics. Trends in Biotechnology. doi:10.1016/50167-7799(02)00038-0).

Primers and a dual-labeled (FAM and TAM or BHQ1) probe specific to adenine phosphoribosyl transferase (APRT) from Solanum tuberosum are used to amplify APRT as an endogenous control. APRT was chosen based on its stability during various biotic and abiotic stress studies in potato using Real-Time PCR (Nicot, N., Hausman, J.-F., Hoffmann, L., & Evers, D. (2005). Housekeeping gene selection for real-time RT-PCR normalization in potato during biotic and abiotic stress. Journal of Experimental Botany, 56(421), 2907-14. doi:10.1093/jxb/eri285). However, any appropriate control gene can be utilized.

Forward and reverse primers, along with dual-labeled probes specific for the APRT control or for each event are listed in Table 7.

TABLE 7
Oligonucleotide Sequences Used in qPCR Assays

| Assay | | SEQ ID |
|---|---|---|
| name | Oligonucleotide sequences | NO |
| Adenine | Forward Primer 5′-GAACCGGAGCAGGTGAAGAA-3′ | 49 |
| Phosphoribosyl | Reverse Primer 5′-GAAGCAATCCCAGCGATACG-3′ | 50 |
| Transferase | Probe with labels and LNAs 5′-6FAM-cgc[+C]tc[+A]tg[+A]tc[+C]gatt-BHQ1-3′ | 51 |
| (APRT) | Probe sequence 5′-cgcctcatgatccgatt-3′ | |
| E12 | Forward Primer 5′-GGGAGTAGGCTTTATACC-3′ | 52 |
| | Reverse Primer 5′-CCTTGAATGATTTGATATAAGAAG-3′ | 53 |
| | Probe with labels and LNAs 5′-6FAM-tcag[+T]ca[+C]tt[+T]gt[+A]ctag-BHQ1-3′ | 54 |
| | Probe sequence 5′ tcagtcactttgtactag 3′ | |
| F10 | Forward Primer 5′-AAGGCAATCTTTCATAAGC-3′ | 55 |
| | Reverse Primer 5′-CACACACTTCGTTTACAC-3′ | 56 |
| | Probe with labels and LNAs 5′-6FAM-tat[+A]tc[+C]tg[+C]tg[+G]acca-BHQ1-3′ | 57 |
| | Probe sequence 5′-tatatcctgctggacca-3′ | |
| J3 | Forward Primer 5′-CCGGTACTCAAATTTGCAA-3′ | 58 |
| | Reverse Primer 5′-GGTGTTCTTAATATCGAGTGTTC-3′ | 59 |
| | Probe with labels and LNAs 5′-6FAM-cca[+C]aa[+G]ct[+A]gg[+A]aactca-BHQ1-3′ | 60 |
| | Probe sequence 5′-ccacaagctaggaaactca-3′ | |
| J55 | Forward Primer 5′-CATGGAATTATTTAGAAACAAAA-3′ | 61 |
| | Reverse Primer 5′-GACGATCAAGAATCTCAATA-3′ | 62 |
| | Probe with labels and LNAs 5′-6FAM-tca[+T]ag[+T]ca[+A]ac[+A]gtcagc-BHQ1-3′ | 63 |
| | Probe sequence 5′-tcatagtcaaacagtcagc-3′ | |
| V11 | Forward Primer 5′-GTGGGCATCAATTTAGTG-3′ | 64 |
| | Reverse Primer 5′-GGAGAGTAATAGCCTTAGTA-3′ | 65 |
| | Probe with labels and LNAs 5′-6FAM-cctcta[+T]ac[+C]tc[+T]ga[+T]aat-BHQ1-3′ | 66 |
| | Probe sequence 5′-cctctatacctctgataat-3′ | |
| | Forward Primer 5′-TAGCCAAGCATGACTTAC-3′ | 67 |
| | Reverse Primer 5′-CACACACTTCGTTTACAC-3′ | 68 |
| | Probe with labels and LNAs 5′-6FAM-atg[+T]at[+G]aa[+T]gg[+C]agaag-BHQ1-3′ | 69 |
| | Probe sequence 5′-atgtatgaatggcagaag-3′ | |
| W8 | Forward Primer 5′-AGGCTTTATACCGAGTTG-3′ | 70 |
| | Reverse Primer 5′-TCAGCTTAATCGAATAAGAAAC-3′ | 71 |
| | Probe with labels and LNAs 5′-6FAM-ACT[+A]CG[+G]TC[+A]GT[+C]ACTT-BHQ1-3′ | 72 |
| | Probe sequence 5′-actacggtcagtcactt-3′ | |
| X17 | Forward Primer 5′-GTCCGAAGGTTGAGAAAA-3′ | 73 |
| | Reverse Primer 5′-CGTGAGACATCATCATAATG-3′ | 74 |
| | Probe with labels and LNAs 5′-6FAM-cgc[+T]tc[+A]gt[+T]ac[+G]gctt-BHQ1-3′ | 75 |
| | Probe sequence 5′-cgcttcagttacggctt-3′ | |
| Y9 | Forward Primer 5′-GACAGCTATCAATATACTTTAGTAA-3′ | 76 |
| | Reverse Primer 5′-TTGCCATACTCATACAGAG-3′ | 77 |
| | Probe with labels and LNAs 5′-6FAM-aat[+G]at[+G]tc[+T]ca[+C]gaccaa-BHQ1-3′ | 78 |
| | Probe sequence 5′-aatgatgtctcacgaccaa-3′ | |
| pSIM1278 | Forward Primer 5′-TCACCAGTATGACTGTTTA-3′ | 79 |
| | Reverse Primer 5′-CTACCACACACTCTCTAG-3′ | 80 |
| | Probe with labels and LNAs 5′-6FAM-aag[+C]tt[+G]tt[+A]ac[+G]aactct-BHQ1-3′ | 81 |
| | Probe sequence 5′-aagcttgttaacgaactct-3′ | |
| | Forward Primer 5′-GCATCATCCATCAAAGTG-3′ | 82 |
| | Reverse Primer 5′-GTCGGTATAATAAGAGAAGGA-3′ | 83 |
| | Probe with labels and LNAs 5′-6FAM-tag[+A]ga[+C]ta[+T]ct[+A]gagtgt-BHQ1-3′ | 84 |
| | Probe sequence 5′-tagagactatctagagtgt-3′ | |
| pSIM1678 | Forward Primer 5′-GAACGGAGGGAGTATGAA-3′ | 85 |
| | Reverse Primer 5′-CCACTTTCAGTTTTGGTTG-3′ | 86 |
| | Probe with labels and LNAs 5′-6FAM-tctt[+T]tc[+A]aa[+G]gg[+G]ccc-BHQ1-3′ | 87 |
| | Probe sequence 5′-tcttttcaaaggggccc-3′ | |
| | Forward Primer 5′-GAAGCCTCTTTTCAAAGG-3′ | 88 |
| | Reverse Primer 5′-ACGGGTTAATCGATAACC-3′ | 89 |
| | Probe with labels and LNAs 5′-6FAM-ccc[+C]aa[+G]tg[+T]ct[+G]aga-BHQ1-3′ | 90 |
| | Probe sequence 5′-ccccaagtgtctgaga-3′ | |

### Example 15

### Efficiency and Linearity of Assays

Two of the hallmarks of optimized qPCR assays are a high PCR efficiency (E) and a linear standard curve measured by the correlation coefficient R². An ideal qPCR reaction has an efficiency of 100% with a slope of −3.32, which correlates with a perfect doubling of PCR product during each cycle. However, slopes between −3.1 and −3.6 with efficiencies between 90 and 110% are generally considered acceptable (Commission, C. A. (2009). Definition of Minimum Performance Requirements for Analytical Methods of GMO Testing European Network of GMO Laboratories (ENGL), (October 2008), 1-8). Efficiency is established by replicated standard curves. Amplification efficiency is determined from the slope of the log-linear portion of the standard curve and is calculated as E=(10^{(-1/slope)}−1)*100 (Bustin, S. A., et al. (2009). The MIQE Guidelines: Minimum Information for Publication of Quantitative Real-Time PCR Experiments. Clinical Chemistry, 55(4), 1-12. doi:10.1373/clinchem.2008.112797) and the R² value is determined by linear regression analysis, which should be ≧0.98 (Bustin et al., 2009).

Table 8 shows the efficiency and linearity of each event-specific and construct-specific qPCR assay.

Each assay demonstrates a high efficiency between the recommended range of 90 to 110% and an R² value of greater than or equal to 0.98.

Data is presented from assays performed on leaf DNA using a combined annealing/extension temperature of 60° C.

TABLE 8
Efficiencies and Linearity of Event-Specific qPCR Assays

| Potato Variety | Assay | Efficiency | Linearity |
|---|---|---|---|
| Russet Burbank | E12 | 98.4% | 0.996 |
| Ranger Russet | F10 | 98.0% | 0.998 |
| Atlantic | J3 | 103.1% | 0.998 |
| Atlantic | J55 | 101.2% | 0.992 |
| Russet Burbank | W8 | 98.4% | 0.994 |
| Snowden | V11 | 104.5% | 0.998 |
| Ranger Russet | X17 | 96.3 | 0.997 |
| Atlantic | Y9 | 93.3 | 0.994 |
| pSIM1278 | Construct* | 104.2% | 0.983 |
| pSIM1278 | Construct\hat{ } | 94.8% | 0.995 |
| pSIM1678 | Construct¹ | 95.7 | 0.996 |

*Determined by using primer/probe set having SEQ ID NOs 79-81
\hat{ }Determined by using primer/probe set having SEQ ID NOs 82-84
¹Determined by using primer/probe set having SEQ ID NOs 85-87 (detects junction between the Vnt1 terminator and the Agp promoter)

### Example 16

### Level of Detection (LOD) of Assays in Potato Leaf DNA

According to the European Network of GMO Laboratories, the “limit of detection is the lowest amount or concentration of analyte in a sample, which can be reliably detected, but not necessarily quantified, as demonstrated by single-laboratory validation” (Commission, 2009). The LOD should detect the analyte at least 95% of the time resulting in ≦5% false negative results. Each of the reported assays was able to reliably detect at least 24 pg of total target DNA greater than 95% of the time. The LOD will vary depending on the source of DNA used since the DNA may be fragmented or contain inhibitors, particularly polysaccharides, as is seen frequently in DNA isolated from potato-based food products. Qualitative and quantitative analysis can reach <0.1% GMO, provided sufficient DNA is used in the PCR reaction.

Table 9 shows the level of detection of each event-specific and construct-specific qPCR assay. Each assay reliably amplified at least 24 pg of potato leaf DNA between 34 and 35 cycles. Data is presented from assays performed using an annealing/extension temperature of 60° C.

TABLE 9
Level of Detection of Event-Specific qPCR Assays

| Assay | Mean Cq at LOD |
|---|---|
| E12 | 35.29 |
| F10 | 35.21 |
| J3 | 34.62 |
| J55 | 35.32 |
| W8 | 35.99 |
| V11 | 35.04 |
| X17 | 35.96 |
| Y9 | 32.85 |
| pSIM1278 Construct* | 34.42 |
| pSIM1278 Construct\hat{ } | 34.78 |
| pSIM1678 Construct¹ | 34.17 |

*Determined by using primer/probe set having SEQ ID NOs 79-81
\hat{ }Determined by using primer/probe set having SEQ ID NOs 82-84
¹Determined by using primer/probe set having SEQ ID NOs 85-87

### Example 17

### Robustness of Event-Specific qPCR Assays

The robustness of a method is a measure of its ability to remain unaffected by small changes in the experimental conditions of an assay. For example, the PCR assays should be able to be performed on different thermal cycler models, by different users and with small deviations in temperature profiles or DNA polymerases. It is generally accepted that the assays should not deviate more than ±30% under these conditions (Commission, 2009). We determined the efficiency and linearity of each assay across a four degree range of combined annealing/extension temperatures from 58° C. to 61° C. (Table 10). Further robustness of the assays is determined by external validation of assay performance by outside laboratories using different users, thermal cyclers and polymerases.

Table 10 shows the efficiency and linearity of each assay over a four degree combined annealing/extension temperature range. With the exception of F10 at 58° C., all of the assays performed well with efficiencies between 90-110% and R² values ≧0.98 over the entire range.

TABLE 10
Efficiencies and Linearity of Event-Specific
qPCR Assays Over a 4° Temperature Range

| | Efficiency (%)/Linearity | | | |
|---|---|---|---|---|
| Assay | 58° C. | 59° C. | 60° C. | 61° C. |
| E12 | 101.9/0.988 | 101.2/0.991 | 98.4/0.996 | 104.0/0.991 |
| F10 | 84.8/0.985 | 95.3/0.980 | 98.0/0.998 | 99.3/0.988 |
| J3 | 99.1/0.994 | 100.6/0.994 | 103.1/0.998 | 101.7/0.997 |
| J55 | 101.1/0.995 | 99.9/0.988 | 101.2/0.992 | 100.9/0.991 |
| W8 | 99.1/0.994 | 100.6/0.994 | 98.4/0.994 | 104.7/0.993 |
| V11 | 93.6/0.997 | 100.0/0.992 | 104.5/0.998 | 95.8/0.994 |
| X17 | 93.1/0.991 | 98.9/0.998 | 103.9/0.974 | 95.3/0.997 |
| Y9 | 97.4/0.991 | 95.2/0.996 | 95.1/0.996 | 101/0.981 |
| pSIM1278 | 104.8/0.992 | 104.0/0.996 | 104.2/0.983 | 95.8/0.994 |
| Construct* | | | | |
| pSIM1278 | 97.6/0.997 | 100.9/0.994 | 94.8/0.995 | 95.8/0.994 |
| Construct\hat{ } | | | | |
| pSIM1678 | 88.3/0.978 | 95.7/0.994 | 93.3/0.996 | 104.4/0.986 |
| Construct¹ | | | | |

*Determined by using primer/probe set having SEQ ID NOs 79-81
\hat{ }Determined by using primer/probe set having SEQ ID NOs 82-84
¹Determined by using primer/probe set having SEQ ID NOs 85-87

### Example 18

### Specificity of Line-Specific Primers and Probes

The specificity of each primer and probe set was assessed by performing qPCR with DNA from each event (E12, F10, J3, J55, W8, V11, X17, and Y9) at three concentrations of DNA (25 ng, 250 pg, and 50 pg) and each commercial variety (Atlantic, Ranger Russet, Burbank and Snowden) using 25 ng of DNA.

The primers used for detecting E12, J3, J55, W8, V11, X17, and Y9 exhibited no false positives or negatives at any of the concentrations tested.

The primers used for F10 amplified a single technical representative of the 25 ng E12 sample after 39 cycles representing a false positive rate of 2.3% which is within the 5% acceptable range.

The primers used for pSIM1278 construct amplified a single technical representative from two different wild type varieties after 41 cycles. In order to improve this assay, the PCR cycling parameters were adjusted and samples were retested. The new parameters resulted in no false positives for pSIM1278 or pSIM1678.

The qPCR reaction set-up and qPCR cycling conditions for the events (E12, F10, J3, J55, W8, V11, X17, and Y9) and constructs (pSIM1278 and pSIM1678) are shown in Tables 11-13.

TABLE 11
PCR Reaction Setup and Conditions (20 μL total volume)

| Reagent | Final Concentration | 1X Sample (μL) |
|---|---|---|
| 2X PerfeCTa ® qPCR | 1X | 10 |
| ToughMix ® (Quanta)* | | |
| Forward primer (10 μM) | 0.5 μM | 1 |
| Reverse primer (10 μM) | 0.5 μM | 1 |
| Probe (10 μM) | 0.2 μM | 0.4 |
| H₂O | | q.s. 20 μL |
| Template DNA | varies | 2 μL |

*Master mix must contain components to reduce PCR inhibition

TABLE 12
PCR Thermal Cycling Conditions E12,
F10, J3, J55, W8, V11, X17, and Y9

| Cycles | Temperature (° C.) | Time (sec) |
|---|---|---|
| 1 | 95 | 600 |
| 45 | 95 | 15 |
| | 60 | 60 |

TABLE 13
PCR Thermal Cycling Conditions pSIM1278 and pSIM1678

| Cycles | Temperature (° C.) | Time (sec) |
|---|---|---|
| 1 | 95 | 600 |
| 40 | 95 | 15 |
| | 60 | 15 |
| | 72 | 10 |

The methods described allow for detection of Innate™ product in potatoes and potato products using qPCR to detect the pSIM1278 construct common to all Innate™ events (including GEN1 and GEN2) using a standard curve derived from freeze-dried Innate™ reference material. In addition, a set of primers and probes were provided that uniquely detect each event qualitatively with a limit of detection <0.1% GMO.

Further, the methods described allow for detection of Innate™ product in potatoes and potato products using qPCR to detect the pSIM1678 construct common to all GEN2 Innate™ events (i.e. W8, X17, and Y9) using a standard curve derived from freeze-dried Innate™ reference material.

### Example 19

### Internal Validation of the Method in Food Mixes

A number of food mixes were prepared to test the developed DNA isolation method. Our internal testing focused on the isolation of DNA from all food matrices from either Atlantic J3 (chip variety) or Ranger Russet F10 (fry variety). Collectively, these two events cover all matrices under consideration (tubers, fries, chips, and flakes). Ground Innate™ food products were mixed into commercial variety food products as described in Table 14. Similar results are expected for all events.

TABLE 14
Food Mixes Produced

| Line | Food Product | Percent Innate ™ Food |
|---|---|---|
| J3 | Freeze-dried Tuber | 0.2/0.5/1.0 |
| F10 | Freeze-dried Tuber | 0.2/0.5/1.0 |
| F10 | Freeze-dried Par Fries | 0.2/0.5/1.0 |
| J3 | Chip | 5.0/10.0 |
| F10 | Flake | 2.5/8.0/15.0 |

Two to three independent DNA isolations were performed on each food mix using the QIAamp Fast DNA Stool Mini Kit from Qiagen as described in Example 12. Table 15 shows the overall DNA concentration and yield from 600 mg fry, flake or chip mix, or 300 mg of tuber.

To ensure that the DNA was of sufficient quality to be used in qPCR, each DNA isolation was run in triplicate with the appropriate set of primers and probes. 2 μl of each DNA isolation were used in each reaction and the endogenous reference gene, APRT, was used as a positive control.

TABLE 15
Average DNA Concentration and Yield
Obtained for Each Food Type

| | Average DNA | Average Total |
|---|---|---|
| | Concentration | DNA Yield |
| Sample | (ng/μL) | (ng) |
| F10 Tuber | 3.2 | 648 |
| J3 Tuber | 6.1 | 610 |
| F10 Fry | 2.5 | 75 |
| J3 Chip | 3.2 | 158 |
| F10 Flake | 5.3 | 160 |

The DNA isolation method produced DNA of sufficient concentration and yield to be used for subsequent qPCR analysis in each of the above food products. The results from the subsequent qPCR analysis can be found in FIG. 7.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

It should be understood that the above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

### REFERENCES

All Chawla, R., Shakya, R., and Rommens, C. M. (2012). Tuber Specific Silencing of Asparagine Synthetase-1 Reduces the Acrylamide-forming Potential of Potatoes Grown in the Field without Affecting Tuber Shape and Yield. Plant Biotechnology Journal 10, 913-924.
Garbarino, J. E., and Belknap, W. R. (1994). Isolation of a Ubiquitin-ribosomal Protein Gene (ubi3) from Potato and Expression of its Promoter in Transgenic Plants. Plant Molecular Biology 24, 119-127.
Garbarino, J. E., Oosumi, T., and Belknap, W. R. (1995). Isolation of a polyubiquitin promoter and its expression in transgenic potato plants. Plant Physiol. 109, 1371-1378.
Simpson, J., Timko, M. P., Cashmore, A. R., Schell, J., van Montagu, M., and Herrera-Estralla, L. (1985) Light-inducible and Tissue-specific Expression of a Chimaeric Gene under Control of the 5′-flanking Sequence of a Pea Chlorophyll a/b-Binding Protein Gene. EMBO J. 4, 2723-2729
Smigocki A C, Owens L D (1988) Cytokinin gene fused with a strong promoter enhances shoot organogenesis and zeatin levels in transformed plant cells. P Natl Acad Sci USA, 85: 5131-5135.
Van Haaren, M. J. J., Sedee, N. J. A, de Boer, H. A., Schilperoort, R. A., and Hooykaas, P. J. J. (1989). Mutational Analysis of the Conserved domains of a T-region Border Repeat of Agrobacterium tumafaciens. Plant Molecular Biology 13, 523-531.

What is claimed is:

1. A quantitative PCR method for detecting the presence of a plant transformation event in a nucleic acid sample, comprising:
a) combining: i) a pair of forward and reverse nucleotide primers, ii) a nucleotide probe, and iii) a target nucleotide sequence from said sample comprising a non-naturally occurring nucleotide junction to be detected;
wherein the nucleotide probe binds to the non-naturally occurring nucleotide junction, or a sequence indicative of the presence of the non-naturally occurring nucleotide junction; and
b) detecting the target nucleotide sequence from said sample.

2. The method of claim 1, wherein the non-naturally occurring nucleotide junction results from a plant transformation event selected from the group consisting of: E12, F10, J3, J55, V11, W8, X17, Y9, or combinations thereof.

3. The method of claim 1, wherein the target nucleotide sequence comprises at least one nucleotide sequence selected from the group consisting of: SEQ ID NOs: 1-48.

4. The method of claim 1, wherein the pair of forward and reverse nucleotide primers and the nucleotide probe are selected from the group consisting of SEQ ID NOs: 52-90.

5. The method of claim 1, wherein the forward nucleotide primer comprises SEQ ID NO: 52 and the reverse nucleotide primer comprises SEQ ID NO: 53 and the nucleotide probe comprises SEQ ID NO: 54.

6. The method of claim 1, wherein the nucleotide probe binds the left or right non-naturally occurring nucleotide junction of an E12 event.

7. The method of claim 1, wherein the forward nucleotide primer comprises SEQ ID NO: 55 and the reverse nucleotide primer comprises SEQ ID NO: 56 and the nucleotide probe comprises SEQ ID NO: 57.

8. The method of claim 1, wherein the nucleotide probe binds the left or right non-naturally occurring nucleotide junction of an F10 event.

9. The method of claim 1, wherein the forward nucleotide primer comprises SEQ ID NO: 58 and the reverse nucleotide primer comprises SEQ ID NO: 59 and the nucleotide probe comprises SEQ ID NO: 60.

10. The method of claim 1, wherein the nucleotide probe binds the left or right non-naturally occurring nucleotide junction of a J3 event.

11. The method of claim 1, wherein the forward nucleotide primer comprises SEQ ID NO: 61 and the reverse nucleotide primer comprises SEQ ID NO: 62 and the nucleotide probe comprises SEQ ID NO: 63.

12. The method of claim 1, wherein the nucleotide probe binds the left or right non-naturally occurring nucleotide junction of a J55 event.

13. The method of claim 1, wherein the forward nucleotide primer comprises SEQ ID NO: 64 or 67 and the reverse nucleotide primer comprises SEQ ID NO: 65 or 68 and the nucleotide probe comprises SEQ ID NO: 66 or 69.

14. The method of claim 1, wherein the nucleotide probe binds the left or right non-naturally occurring nucleotide junction of a V11 event.

15. The method of claim 1, wherein the forward nucleotide primer comprises SEQ ID NO: 70 and the reverse nucleotide primer comprises SEQ ID NO: 71 and the nucleotide probe comprises SEQ ID NO: 72.

16. The method of claim 1, wherein the nucleotide probe binds the left or right non-naturally occurring nucleotide junction of a W8 event.

17. The method of claim 1, wherein the forward nucleotide primer comprises SEQ ID NO: 73 and the reverse nucleotide primer comprises SEQ ID NO: 74 and the nucleotide probe comprises SEQ ID NO: 75.

18. The method of claim 1, wherein the nucleotide probe binds the left or right non-naturally occurring nucleotide junction of an X17 event.

19. The method of claim 1, wherein the forward nucleotide primer comprises SEQ ID NO: 76 and the reverse nucleotide primer comprises SEQ ID NO: 77 and the nucleotide probe comprises SEQ ID NO: 78.

20. The method of claim 1, wherein the nucleotide probe binds the left or right non-naturally occurring nucleotide junction of a Y9 event.

21. The method of claim 1, wherein the forward nucleotide primer comprises SEQ ID NO: 79 or 82 and the reverse nucleotide primer comprises SEQ ID NO: 80 or 83 and the nucleotide probe comprises SEQ ID NO: 81 or 84.

22. The method of claim 1, wherein the nucleotide probe binds an internal non-naturally occurring nucleotide junction associated with pSIM1278.

23. The method of claim 1, wherein the forward nucleotide primer comprises SEQ ID NO: 85 or 88 and the reverse nucleotide primer comprises SEQ ID NO: 86 or 89 and the nucleotide probe comprises SEQ ID NO: 87 or 90.

24. The method of claim 1, wherein the nucleotide probe binds an internal non-naturally occurring nucleotide junction associated with pSIM1678.

25. The method of claim 1, wherein the nucleic acid sample is from a potato plant, or potato plant part, or potato derived food product.

26. The method of claim 1, wherein the nucleic acid sample is from a potato plant part selected from the group consisting of: potato flowers, potato tepals, potato petals, potato sepals, potato anthers, potato pollen, potato seeds, potato leaves, potato petioles, potato stems, potato roots, potato rhizomes, potato stolons, potato tubers, potato shoots, potato cells, potato protoplasts, potato plant tissues, and combinations thereof.

27. The method of claim 1, wherein the nucleic acid sample is from a potato derived food product selected from the group consisting of: a potato processed food product, a potato livestock feed material, French fries, potato chips, dehydrated potato material, potato flakes, potato granules, potato protein powder, potato starch, potato flour, instant potato products, and combinations thereof.

28. The method of claim 1, wherein the nucleic acid sample is from a potato derived food product and wherein the presence of at least one plant transformation event selected from the group consisting of E12, F10, J3, J55, V11, W8, X17, and Y9 is able to be detected in the food product.

29. The method of claim 1, wherein the nucleic acid sample is from a potato derived food product and wherein the presence of at least one plant transformation event selected from the group consisting of E12, F10, J3, J55, V11, W8, X17, and Y9 is able to be detected in the food product at levels less than 1% of the total food product.

30. The method of claim 1, wherein the nucleic acid sample is from a potato derived food product and wherein the presence of at least one plant transformation event selected from the group consisting of E12, F10, J3, J55, V11, W8, X17, and Y9 is able to be detected in the food product at levels ranging from about 0.1% to about 5% of the total food product.

31. An isolated non-naturally occurring nucleic acid junction sequence sharing at least 85% sequence homology to a nucleic acid selected from the group consisting of SEQ ID NOs: 1-48.

32. The isolated non-naturally occurring nucleic acid junction sequence of claim 31 sharing at least 95% sequence homology to a nucleic acid selected from the group consisting of SEQ ID NOs: 1-48.

33. The isolated non-naturally occurring nucleic acid junction sequence of claim 31 sharing 100% sequence homology to a nucleic acid selected from the group consisting of SEQ ID NOs: 1-48.

34. An isolated non-naturally occurring nucleic acid probe sequence capable of hybridizing under stringent conditions to a nucleic acid selected from the group consisting of SEQ ID NOs: 1-48.

35. An isolated non-naturally occurring nucleic acid probe sequence sharing at least 85% sequence homology to a nucleic acid selected from the group consisting of SEQ ID NOs: 54, 57, 60, 63, 66, 69, 72, 75, 78, 81, 84, 87, and 90.

36. The isolated non-naturally occurring nucleic acid probe sequence of claim 35 sharing at least 95% sequence homology to a nucleic acid selected from the group consisting of SEQ ID NOs: 54, 57, 60, 63, 66, 69, 72, 75, 78, 81, 84, 87, and 90.

37. The isolated non-naturally occurring nucleic acid probe sequence of claim 35 sharing 100% sequence homology to a nucleic acid selected from the group consisting of SEQ ID NOs: 54, 57, 60, 63, 66, 69, 72, 75, 78, 81, 84, 87, and 90.

38. An isolated non-naturally occurring nucleic acid primer or probe sequence sharing at least 95% sequence homology to a nucleic acid selected from the group consisting of SEQ ID NOs: 52-90.

39. A kit comprising the nucleic acid primer or probe sequence according to claim 38.
